(19) United States
(12) Patent Application Publication
(10) Pub. No.: US 2026/0241046 A1
(43) Pub. Date: Aug. 20, 2026

(54) PROSTATE-SPECIFIC MEMBRANE ANTIGEN RECEPTOR-TARGETING RADIOPHARMACEUTICALS AND USES THEREOF

(71) Applicants: Nuclidium AG, Basel (CH); University of Basel, Basel (CH)

(72) Inventors: Leila Jaafar-Thiel, Basel (CH); Francesco De Rose, Basel (CH); Melpomeni Fani, Basel (CH)

(21) Appl. No.: 19/375,108

(22) Filed: Oct. 30, 2025

Related U.S. Application Data
(63) Continuation of application No. PCT/US2025/021198, filed on Mar. 24, 2025.
(60) Provisional application No. 63/568,830, filed on Mar. 22, 2024.

(51) Int. Cl. A61K 51/04; A61K 47/02; A61K 47/22; A61P 35/00

(52) U.S. Cl. CPC A61K 51/0497; A61K 47/02; A61K 47/22; A61K 51/0482; A61P 35/00

(57) ABSTRACT

The present disclosure relates to pharmaceutical compositions comprising [⁶¹Cu]Cu-NODAGA-PSMA I&T or a pharmaceutically acceptable salt thereof. The present disclosure also relates to methods of imaging subjects suspected of having or diagnosed with a prostate-specific membrane antigen (PSMA)-expressing tumor by administering the pharmaceutical compositions described herein and generating one or more radiographic images of the subject.

### 1. BACKGROUND

Prostate-specific membrane antigen (PSMA) is a transmembrane protein which is highly upregulated in prostate cancer cells. Upregulation may exceed 100-fold in prostate cancer cells, making PSMA a target for detection of prostate cancer and prostate cancer metastases. [⁶⁸Ga]Ga-PSMA-11 (Illuccix®, Locametz®), ¹⁸F-Piflufolastat (Pylarify®), and ¹⁸F-Flotufolastat (Posluma®) have been FDA-approved for PSMA-targeted imaging in patients with prostate cancer. These agents are composed of a PSMA-targeting binding agent and a positron emitting isotope which allows localization of the agent using Positron Emission Tomography (PET). PSMA-targeted PET radiotracers allow detection for staging of patients with newly diagnosed prostate cancer at high risk of metastases, as well as detection and localization of disease sites in patients with biochemical recurrence as measured by increase of serum Prostate Specific Antigen (PSA).

The short half-life of ¹⁸F (1.8 hours) translates to a limited geographic distribution range of ¹⁸F-labeled radiotracers to sites reachable within 5-6 hours after production, or about 350 miles.

The distribution range for radiotracers made with ⁶⁸Ga is considerably more limited. Use of ⁶⁸Ga-labelled imaging agents is hampered by (i) the expense and limited production capacity of ⁶⁸Ge/⁶⁸Ga-generators, (ii) the short half-life of ⁶⁸Ga (t_{1/2}=1.13 h), which prevents the shipment of the radiotracer from central producers to smaller centers that are located beyond 2 hours' distance, and (iii) the high-energy of ⁶⁸Ga (Emax=1.9 MeV), which limits spatial resolution of reconstructed PET imaging.

There is a need for improved methods of generating radiographic images of subjects with PSMA-expressing tumors for the diagnosis, monitoring, and ultimately, treatment of cancer patients.

### 2. SUMMARY

In one aspect, the present disclosure provides a pharmaceutical composition comprising a radiotracer that has the structure

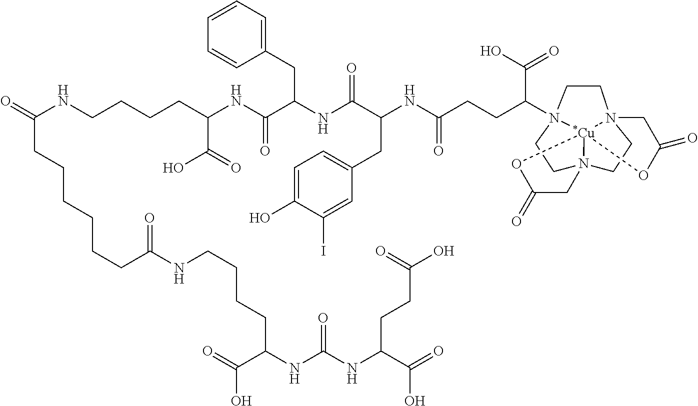

or is a pharmaceutically acceptable salt thereof;
   wherein
*Cu is selected from ⁶¹Cu, ⁶⁴Cu, and ⁶⁷Cu, particularly ⁶¹Cu.
the pharmaceutical composition is characterized by a radiotracer radiochemical purity of ≥97% at 12 hours after end of synthesis and optionally one or more of:
   a [*Cu]Cu, particularly [⁶¹Cu]Cu, radionuclidic purity at end of synthesis of ≥97%,
   a radiocobalt activity content at end of synthesis of ≤0.05%,
   a ^{110m}Ag specific activity≤0.1 Bq/g,
   a ^{108m}Ag specific activity≤0.1 Bq/g, or
   a ¹⁰⁹Cd specific activity≤0.1 Bq/g.

In another aspect, the present disclosure provides a method for imaging a subject comprising:
(a) administering an effective amount of a pharmaceutical composition to a subject suspected of having or diagnosed with a PMSA-expressing tumor, wherein the pharmaceutical composition comprises a radiotracer that has the structure

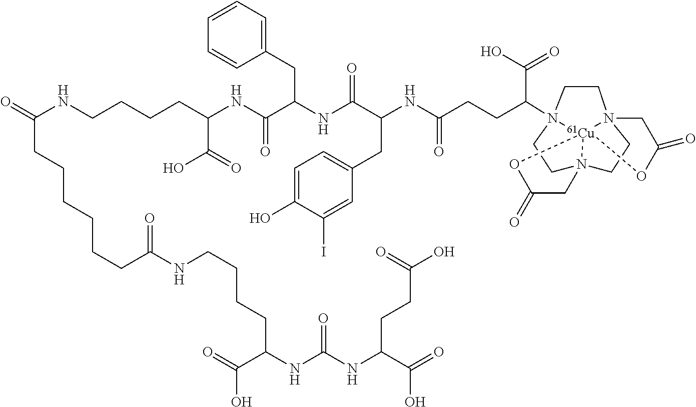

or is a pharmaceutically acceptable salt thereof;
wherein the pharmaceutical composition is characterized by a radiotracer radiochemical purity of ≥97% at 12 hours after end of synthesis and optionally one or more of:
   a [⁶¹Cu]Cu radionuclidic purity at end of synthesis of ≥97%,
   a radiocobalt activity content at end of synthesis of ≤0.05%,
   a ^{110m}Ag specific activity≤0.1 Bq/g,
   a ^{108m}Ag specific activity≤0.1 Bq/g, or
   a ¹⁰⁹Cd specific activity≤0.1 Bq/g; and
(b) generating one or more radiographic images of the subject.

In another aspect, the present disclosure provides a method for determining a subject's response to a cancer treatment comprising:
(a) administering an effective amount of a pharmaceutical composition to a subject diagnosed with a PSMA-expressing tumor at an earlier time point and at a later time point, wherein the pharmaceutical composition comprises a radiotracer that has the structure

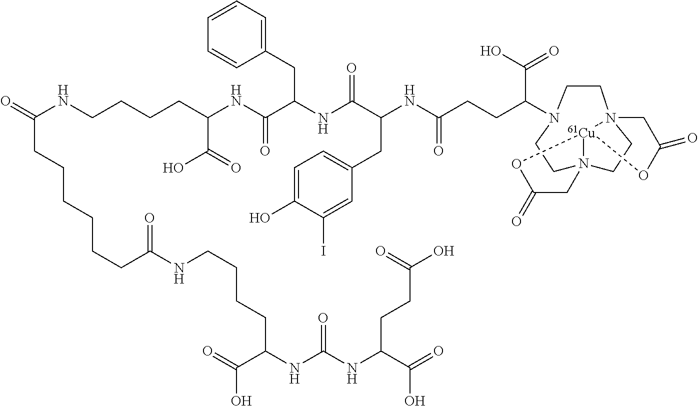

or is a pharmaceutically acceptable salt thereof;
wherein the pharmaceutical composition is characterized by a radiotracer radiochemical purity of ≥97% at 12 hours after end of synthesis and optionally one or more of:
   a [⁶¹Cu]Cu radionuclidic purity at end of synthesis of ≥97%,
   a radiocobalt activity content at end of synthesis of ≤0.05%,
   a ^{110m}Ag specific activity≤0.1 Bq/g,
   a ^{108m}Ag specific activity≤0.1 Bq/g, or
   a ¹⁰⁹Cd specific activity≤0.1 Bq/g;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

In yet another aspect, the present disclosure provides a theranostic method comprising:
(a) administering to a subject an effective amount of a first pharmaceutical composition described herein;
(b) generating one or more images of the subject; and
(c) administering to the subject an effective amount of a second pharmaceutical composition comprising a radiopharmaceutical.

### 3. BRIEF DESCRIPTION OF THE DRAWINGS

These and other features, aspects, and advantages of the present disclosure will become better understood with regard to the following description, and accompanying drawings, where:

FIG. 1 illustrates with increasing magnification homogenous nickel coating having durable adhesion to a niobium coin upon completion of electroplating, as evaluated using a DINOLite digital microscope. Panel A, 20× magnification; panel B, 50× magnification; panel C, 250× magnification.

FIG. 2 shows samples of the coin provided according to the present disclosure with nickel deposited in the center of a niobium backing.

FIG. 3 displays the analysis of ⁶¹Cu purity of [⁶¹Cu]CuCl₂ solution obtained by irradiation of ^{nat}Ni on Nb backing with deuteron beam at 8.4 MeV for 3 h at 50 μA. The curved line corresponds to reduction in % purity of ⁶¹Cu over time and the bars correspond to radiocobalt activity over time.

FIG. 4 displays an analysis of ⁶¹Cu purity of [⁶¹Cu]CuCl₂ solution obtained by irradiation of ⁶⁰Ni on Nb backing with a deuteron beam at 8.4 MeV for 3 h at 50 μA. The curved line corresponds to the reduction in % purity of ⁶¹Cu over time, and the bars correspond to radiocobalt activity over time.

FIG. 5 presents the specific activities of detected impurities in [⁶¹Cu]CuCl₂ solutions produced according to various methods. The ext. coin (Ag, ^{nat}Ni) data was generated by irradiation of a commercially available ^{nat}Ni target on Ag backing. The (Nb, ^{nat}Ni) and (Nb, Ni-61) data were generated based on irradiation of Ni targets (natural and isotopically enriched in ⁶¹Ni, respectively) electroplated according to the present disclosure on high-purity Nb backing. The specific activities were assessed by gamma spectrometry and reported in Bq/g. The data shows that silver and cobalt isotopes were significantly reduced in the [⁶¹Cu]CuCl₂ solution produced by irradiation of Ni targets electroplated according to the present disclosure on high-purity Nb backing.

FIG. 6 shows the significant reduction in the sum of radionuclidic impurities present in a [⁶¹Cu]CuCl₂ solutions produced according to various methods. The ext. coin (Ag, ^{nat}Ni) data was generated based on irradiation of a commercially available ^{nat}Ni target on Ag backing. The (Nb, ^{nat}Ni) and (Nb, Ni-61) data were generated based on irradiation of Ni targets (natural and isotopically enriched in ⁶¹Ni, respectively), electroplated according to the present disclosure on high-purity Nb backing. The radionuclidic impurities were determined by gamma spectrometry and reported in Bq/g (summed radionuclidic impurities). The presented data highlight in particular the reduction of overall impurities in the [⁶¹Cu]CuCl₂ solution when produced in accordance with the present disclosure.

FIG. 7 illustrates the sustained high radionuclidic purity of a [⁶¹Cu]CuCl₂ solution produced according to the present disclosure compared to a commercially available ^{nat}Ni target on a Ag backing (ext. coin (Ag, ^{nat}Ni)). The (Nb, ^{nat}Ni) and (Nb, Ni-61) coins were prepared by electrodeposition according to the present disclosure on high-purity Nb backing. The data was generated using gamma spectrometry and reported in Bq/g providing the summed radionuclidic purities at t=0 h and at t=12 h. The presented data highlight the superior quality of the [⁶¹Cu]CuCl₂ solution when produced by irradiation of Ni targets electroplated according to the present disclosure on high purity Nb backing, where the purity after 12 hours is still well above the purity limits set by pharmacopeia for similar radionuclides for medical use.

FIG. 8 displays chemical impurities, as measured by ICP-MS, of the [⁶¹Cu]CuCl₂ solution when produced by bombardment of ^{nat}Ni vs. ⁶¹Ni when produced by irradiation of Ni targets electroplated according to the present disclosure on high-purity Nb backing.

FIG. 9 shows maximum intensity projections (MIPs) PET/CT images of [⁶¹Cu]Cu-NODAGA-PSMA I&T at 1 hour (dynamic scan) and 4 hours post-injection. Scale 0-7 in standardized uptake value (SUV) in HEK-SST₂ xenografted mice. Scale 0-4 in standardized uptake value (SUV).

FIG. 10 shows MIPs PET images from dynamic acquisition of [⁶¹Cu]Cu-NODAGA-PSMA I&T with and without blocking agent 2-PMPA and [⁶¹Cu]CuCl₂ at 1 hour post-injection in LNCaP xenografted nude mice.

FIG. 11 shows first-in-human administration of [⁶¹Cu]Cu-NODAGA-PSMA I&T in a man with metastatic castration-resistant prostate cancer with disease progression following abiraterone and docetaxel therapy. (A) MIP, (B) PET, (C) CT, and (D) fused PET/CT images demonstrate multifocal osseous (arrows) and hepatic (arrowheads) metastases. The patient has one kidney following a left nephrectomy.

FIG. 12 shows administration of Pylarify and [⁶¹Cu]Cu-NODAGA-PSMA I&T (aka ⁶¹Cu-PSMA) in Subject #1 with bone metastases. More osseous metastases were visualized by [⁶¹Cu]Cu-NODAGA-PSMA I&T, which increased with time after tracer administration. Excretion of tracer into the gallbladder and bowel on 2 and 4 hour images.

FIG. 13 shows administration of Pylarify and [⁶¹Cu]Cu-NODAGA-PSMA I&T (aka ⁶¹Cu-PSMA) in Subject #2 with bone metastases. Same number of metastases were observed on Pylarify and [⁶¹Cu]Cu-NODAGA-PSMA I&T PET/CT images (prostate and left pubic ramus). Excretion of [⁶¹Cu]Cu-NODAGA-PSMA I&T into the gallbladder and bowel increased with time.

FIG. 14 shows administration of Pylarify and [⁶¹Cu]Cu-NODAGA-PSMA I&T (aka ⁶¹Cu-PSMA) in Subject #3 with pelvic nodal metastases. 5 pelvic nodal metastases were seen with [⁶¹Cu]Cu-NODAGA-PSMA I&T PET/CT at 4 hours compared to 2 seen on Pylarify PET/CT.

FIG. 15 shows administration of Pylarify and [⁶¹Cu]Cu-NODAGA-PSMA I&T (aka ⁶¹Cu-PSMA) in Subject #4 with bone metastases. More osseous metastases were observed on [⁶¹Cu]Cu-NODAGA-PSMA I&T PET/CT than Pylarify PET/CT. Number of visualized metastases was greatest at 4-hour imaging time.

FIG. 16 shows administration of Pylarify and [⁶¹Cu]Cu-NODAGA-PSMA I&T (aka ⁶¹Cu-PSMA) in Subject #5 with bone metastases. Equal number of osseous metastases were observed on [⁶¹Cu]Cu-NODAGA-PSMA I&T PET/CT and Pylarify PET/CT. [⁶¹Cu]Cu-NODAGA-PSMA I&T PET/CT SUVs were highest at 4 hours after tracer administration. Background SUVs were higher on Pylarify PET/CT.

FIG. 17 shows administration of Pylarify and [⁶¹Cu]Cu-NODAGA-PSMA I&T (aka ⁶¹Cu-PSMA) in Subject #6 with bone metastases. One avid 0.4 cm left pelvic sidewall lymph node seen on both Pylarify and [⁶¹Cu]Cu-NODAGA-PSMA I&T PET/CT. On 4-hour [⁶¹Cu]Cu-NODAGA-PSMA I&T PET/CT, lesional SUVs were higher, and background SUVs were lower.

FIG. 18 shows administration of Pylarify and [⁶¹Cu]Cu-NODAGA-PSMA I&T (aka ⁶¹Cu-PSMA) in Subject #7 with bone metastases. More osseous metastases were detected on [⁶¹Cu]Cu-NODAGA-PSMA I&T PET/CT, than on Pylarify PET/CT. Excretion in the gallbladder is seen on 4-hour [⁶¹Cu]Cu-NODAGA-PSMA I&T PET/CT images.

FIG. 19 shows administration of Pylarify and [⁶¹Cu]Cu-NODAGA-PSMA I&T (aka ⁶¹Cu-PSMA) in Subject #8 with bone metastases. Solitary avid osseous metastasis were observed in both exams. SUV value for the lesion was highest, and background measurements were lowest, on the 4-hour [⁶¹Cu]Cu-NODAGA-PSMA I&T PET/CT.

FIG. 20 shows AMA test plot obtained plotting at x-axis (logarithmic) the nmol of titrating chelator and at y-axis the % of complexation obtained via radio-TLC.

FIG. 21 shows experimental point corresponding to the lowest value of nmol of chelator in correspondence of which≥95% complexation was achieved (black arrow).

### 4. DETAILED DESCRIPTION

### 4.1. Definitions

When describing the embodiments of the present disclosure, the following terms, if present, have the following meanings, unless otherwise indicated. If not otherwise defined, terms have their customary meaning in the relevant art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, “absorbed dose” refers to the amount of radiation absorbed by an object (e.g., an organ or tissue) or a person. The gray (Gy) is the SI unit of absorbed dose and is defined as the absorption of one joule of energy, in the form of ionizing radiation, per kilogram of matter, i.e., one gray=1 J/kg².

As used herein, “activity” or “radioactivity” refers to a physical quantity defined as the number of radioactive transformations per second that occur in a particular compound or composition that contains one or more radioactive substances. The unit of activity used herein is the becquerel (Bq), which is defined equivalent to reciprocal seconds (1/seconds or s⁻¹).

As used herein, “activity concentration” refers to the total amount of radioactivity per unit volume. In certain embodiments, activity concentration is expressed in Bq/L or magnitudes thereof (e.g., MBq/mL).

As used herein, “radiocobalt activity content” refers to the ratio, expressed as a percentage, of the radioactivity of radiocobalt species in a composition to the total radioactivity of the composition.

As used herein, “end of synthesis” (EoS) refers to the completion of radiolabeling NODAGA-PSMA I&T with [⁶¹Cu]CuCl₂ and subsequent preparation of the pharmaceutical composition, e.g., dilution with saline solution.

As used herein, “effective amount,” “pharmaceutically effective amount,” or “therapeutically effective amount” mean a sufficient amount of the pharmaceutical composition to provide the desired utility when administered to a subject. In certain embodiments, an effective amount includes an amount of pharmaceutical composition sufficient to generate an image of subject. In certain embodiments, an effective amount includes an amount of pharmaceutical composition sufficient to diagnose a disease in a subject. It is understood that for any given case, an appropriate “effective amount” can be determined by one of ordinary skill in the art using routine experimentation. For example, when administered in clinic, such pharmaceutical compositions will contain an amount of active ingredient effective to achieve the desired result (e.g., imaging cancerous tissue).

As used herein, “effective dose” refers to the internationally accepted central radiological risk metric quantity. The calculation of effective dose can be seen as a three-step process. First, the mean absorbed doses to organs and tissues are determined in gray (Gy; J kg⁻¹). Second, the absorbed doses are converted to equivalent doses in sievert (Sv) using tissue/organ radiation weighting factors (wR). The weighting factors were computed from radiation epidemiological data, and take into consideration that some organs (e.g., breasts, stomach, and, lungs) are more radiosensitive than others (e.g., brain and skin). The summing of organ/tissue equivalent doses, each weighted by the appropriate tissue weighting factor (wT), gives the effective dose, in Sv or Sv/MBq (i.e., the effective dose received per unit of activity administered) The effective dose is therefore a risk metric, and not a dosimetric quantity per se.

As used herein, “molar activity” refers to the amount of radioactivity (e.g., number of nuclear disintegrations per second) per unit mole of the radiolabeled compound. In certain embodiments, molar activity is expressed in Bq/mol, e.g., MBq/nmol and is used where the molecular weight of the labelled material is known. The “apparent molar activity” (apparent Am) takes into account the amounts of the radiolabeled (compound A*) and non-radiolabeled radiotracer (compound A), radiolabeled impurities (compound B*) and non-radiolabeled impurities (compound B) and remaining precursor (compound C) present expressed in mol (or μmol). See, e.g., Luurtsema, G., et al. “EANM guideline for harmonisation on molar activity or specific activity of radiopharmaceuticals: impact on safety and imaging quality.” EJNMMI Radiopharm. Chem. 6, 34 (2021).

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium, and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutical composition” refers to a composition suitable for administration to a subject that comprises [⁶¹Cu]Cu-NODAGA-PSMA I&T or a salt thereof and one or more pharmaceutically acceptable excipients. A “pharmaceutically acceptable excipient”, as used herein refers to for example, pharmaceutically, physiologically, acceptable organic or inorganic carrier substances suitable for intravenous administration that do not deleteriously react with the [⁶¹Cu]Cu-NODAGA-PSMA I&T or salt thereof. Exemplary pharmaceutical excipients are known to those of skill in the art.

As used herein, “positive predictive value” or PPV refers to a given method's precision in identifying cancerous lesions/tumors. A method that has a high PPV is more desirable. Mathematically, PPV is the number of true positives (TP) divided by the sum of TP and false positives (FP). In other words, PPV is TP/(TP+FP).

As used herein, “peptide receptor radionuclide therapy” (PRRT) refers to the use of one or more radiotherapeutic agents for the delivery of targeting radiation, usually by a chelated radionuclide, to a specific tissue or receptor associated with cancer. The targeting and selection of certain tissues over another is accomplished by specific peptide hormone receptors expressed or overexpressed on the surface of cancerous biomass.

As used herein, “radiochemical purity” refers to the ratio, given as a percent, of radioactivity from the radionuclide in the pharmaceutical composition (e.g., the desired radionuclide that is chelated in a radiotracer as described herein) to the total radioactivity of the composition that comprises the radiotracer. The majority of the radioactive isotope is attached to the tracer construct and is not free or attached to another chemical entity as these forms may have a different biodistribution. Radiochemical purity (RCP) measurements establish the content of impurities labelled with the same radionuclide used to prepare a radiopharmaceutical, but with a different chemical form.

Radiochemical purity is determined according to methods well known to those of skill in the art, e.g., radio-HPLC or iTLC. As is understood in the art, determination of radiochemical purity is not strictly quantitative, and it is calculated as the ratio between the peak area of the desired radiotracer and the overall area of all the detected peaks in the radiochromatogram (corrected for decay). The instrument used to determine radiochemical purity with HPLC (radio-HPLC) is a radiometric detector (radiodetector), which has an in-line detector connected in series with a UV or other physicochemical detector. The radiometric detector can be a Geiger-Müller probe, a scintillation detector, or a PIN diode. As compared with radio-HPLC it has the big advantage that all applied radioactivity is detected and there are no concerns with recovery. The radiochemical purity is reported at a particular time point, e.g., a certain time after end of synthesis (EoS, described herein). In certain embodiments, a radiotracer radiochemical purity is measured at 3 hours after end of synthesis of the radiotracer, at 6 hours after end of synthesis of the radiotracer, at 9 hours after end of synthesis of the radiotracer, or at 12 hours after end of synthesis of the radiotracer.

As used herein, “radionuclidic purity” refers to the ratio, expressed as a percentage, of the radioactivity of a particular radionuclide to the total radioactivity of the sample, e.g., the starting material used to prepare a radiotracer. In certain embodiments, radionuclidic purity can be determined by high resolution gamma spectroscopy (e.g., high-purity germanium (HPGe) detector) on a sample after expiration, e.g. >8 hours or >3 weeks) and is then extrapolated (e.g., using the TENDL-2019 database according to procedures well known in the art), and reported herein as the value at the end of synthesis (e.g., EoB+2 hours) of the radionuclide.

As used herein, “radionuclidic impurities” refer to all non-⁶¹Cu radionuclides in a composition.

As used herein, “radiotracer” refers to a compound of the present disclosure comprising a radionuclide or radioisotope. It is understood herein that when a compound, e.g., a radiotracer, is described as comprising a particular radioisotope or radionuclide (e.g., ⁶¹Cu) that the compound is isotopically enriched in that isotope at the indicated position.

As used herein, “sensitivity” refers to the ability of a given method to accurately identify cancerous lesions/tumors. A method that has high sensitivity is more desirable. Mathematically, sensitivity is the number of true positives (TP) divided by the sum of the number of TPs and false positives (FP). In other word, sensitivity (Se)=TP/(TP+FN).

As used herein, “specific activity” refers to the activity of a radionuclide per unit mass or per unit volume. In certain embodiments, specific activity refers to the activity of a radionuclide per unit mass of the radiotracer. In certain embodiments, specific activity refers to the activity of a radionuclide per unit mass of the [⁶¹Cu]CuCl₂ solution used to radiolabel the NODAGA-PSMA I&T. In certain embodiments, specific activity refers to the activity of a radionuclide per unit volume of the [⁶¹Cu]CuCl₂ solution used to radiolabel the NODAGA-PSMA I&T. In certain embodiments, specific activity is measured at EoS.

As used herein, “subject” refers to the person or organism to which the composition is, or is intended to be, administered. As such, subjects of the present disclosure may include but are not limited to mammals, e.g., humans and other primates, such as chimpanzees and other ape and monkey species. In preferred embodiments the subject are humans. The term subject includes a person or organism of any age, weight, or other physical characteristic, including an adult, an adolescent, a child, an infant or a newborn.

### 4.2. Pharmaceutical Compositions

In one aspect, the present disclosure provides pharmaceutical compositions comprising a radiotracer ([*Cu]Cu-NODAGA-PSMA I&T) that has the structure

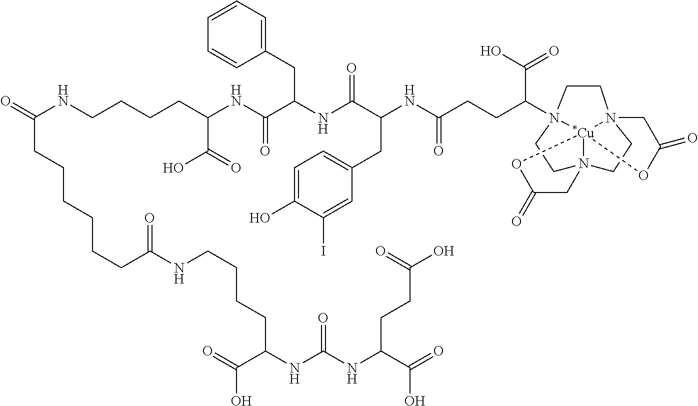

or is a pharmaceutically acceptable salt thereof, wherein *Cu is selected from ⁶¹Cu, ⁶⁴Cu, and ⁶⁷Cu, particularly ⁶¹Cu.

The pharmaceutical compositions are useful in the methods described herein, e.g., for generating radiographic images of subjects diagnosed with or suspected of having a PSMA-expressing tumor following administration of the pharmaceutical composition, e.g., by intravenous (i.v.) administration. Copper 61, also known as ⁶¹Cu, has a half-life of 3.3 hours, which permits a broader distribution range and usage of ⁶¹Cu-labelled radiotracers than ⁶⁸Ga-labelled or ¹⁸F-labelled imaging agents. For example, pharmaceutical compositions containing ⁶¹Cu-labelled radiotracers of the present disclosure have a distribution range of more than 700 miles, allowing less than 10 production sites to provide radioisotopes to the entire continental United States.

In certain embodiments, the radiotracer has the structure

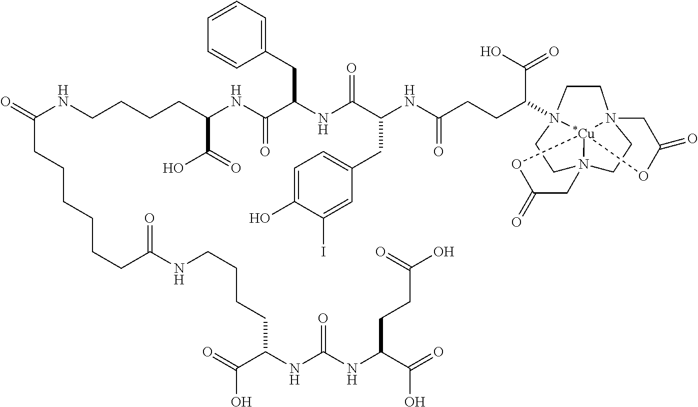

or is a pharmaceutically acceptable salt thereof, wherein *Cu is selected from ⁶¹Cu, ⁶⁴Cu, and ⁶⁷Cu, particularly ⁶¹Cu.

In certain embodiments, the pharmaceutical composition comprises the radiotracer in an amount≥1 μg, e.g., ≥10 μg, ≥20 μg, ≥30 μg, ≥40 μg, ≥50 μg, ≥60 μg, ≥70 μg, or ≥80 μg.

In certain embodiments, the pharmaceutical composition comprises the radiotracer in an amount from 1 μg to 100 μg, e.g., from 20 μg to 90 μg, from 20 μg to 80 μg, from 20 μg to 70 μg, from 20 μg to 60 μg, from 20 μg to 50 μg, from 20 μg to 40 μg, from 20 μg to 30 μg, from 30 μg to 100 μg, from 30 μg to 90 μg, from 30 μg to 80 μg, from 30 μg to 70 μg, from 30 μg to 60 μg, from 30 μg to 50 μg, from 30 μg to 40 μg, from 40 μg to 100 μg, from 40 μg to 90 μg, from 40 μg to 80 μg, from 40 μg to 70 μg, from 40 μg to 60 μg, from 40 μg to 50 μg, from 50 μg to 100 μg, from 50 μg to 90 μg, from 50 μg to 80 μg, from 50 μg to 70 μg, from 50 μg to 60 μg, from 60 μg to 100 μg, from 60 μg to 90 μg, from 60 μg to 80 μg, from 60 μg to 70 μg, from 70 μg to 100 μg, from 70 μg to 90 μg, from 70 μg to 80 μg, from 80 μg to 100 μg, from 80 μg to 90 μg, or from 90 μg to 100. In certain embodiments, the pharmaceutical composition comprises 1 μg to 50 μg of the radiotracer, e.g., from 1 μg to 30 μg, from 1 μg to 10 μg, or from 1 μg to 5 μg.

In certain embodiments, the pharmaceutical composition is in the form of a liquid, e.g., a solution, such as an aqueous solution.

In certain embodiments, the pharmaceutical composition comprises an aqueous vehicle, i.e., a medium or carrier comprising at least a minimal amount of water, in which all the other components are dissolved. Exemplary aqueous vehicles include, e.g., deionized water, saline, phosphate buffer, citrate buffer, malate buffer, tartrate buffer, balanced salt solution, salts of organic acids, combinations of organic acids and salts of organic acids (e.g., tribasic sodium citrate and citric acid, malic acid and sodium malate, and potassium sodium tartrate and tartaric acid), and combinations thereof. In certain embodiments, the aqueous vehicle comprises saline solution, e.g., isotonic saline solution.

In certain embodiments, the pharmaceutical composition has a total volume of 2 to 15 mL, e.g., from 2 mL to 14 mL, from 2 mL to 12 mL, from 2 mL to 10 mL, from 2 mL to 8 mL, from 2 mL to 6 mL, from 2 mL to 4 mL, from 4 mL to 15 mL, from 4 mL to 14 mL, from 4 mL to 12 mL, from 4 mL to 10 mL, from 4 mL to 8 mL, from 4 mL to 6 mL, from 6 mL to 15 mL, from 6 mL to 14 mL, from 6 mL to 12 mL, from 6 mL to 10 mL, from 8 mL to 15 mL, from 8 mL to 14 mL, from 8 mL to 12 mL, from 8 mL to 10 mL, from 12 mL to 15 mL, or from 12 mL to 14 mL. In certain embodiments, the pharmaceutical composition has a total volume from 2 mL to 5 mL or from 8 to 12 mL.

The total volume of the pharmaceutical composition comprises one or more unit doses for administration to the subject in the methods described herein, e.g., one dose, two doses, or three or more doses. In certain embodiments, the pharmaceutical composition comprises from 2 mL to 5 mL, corresponding to one unit dose for administration. In certain embodiments, the pharmaceutical composition comprises from 8 mL to 12 mL, corresponding to two unit doses for administration.

### 4.2.1. Properties

In certain embodiments, the pharmaceutical composition has an activity of ≥25 MBq, e.g., ≥30 MBq, ≥50 MBq, ≥75 MBq, ≥100 MBq, ≥150 MBq, ≥200 MBq, ≥250 MBq, ≥300 MBq, ≥350 MBq, ≥400 MBq, ≥450 MBq, ≥500 MBq, ≥750 MBq, or ≥1,000 MBq.

In certain embodiments, the pharmaceutical composition has an activity from 25 MBq to 1,500 MBq, e.g., from 25 MBq to 1,000 MBq, from 25 MBq to 750 MBq, from 25 MBq to 500 MBq, from 25 MBq to 300 MBq, from 25 MBq to 200 MBq, from 25 MBq to 100 MBq, from 50 MBq to 1,500 MBq, from 50 MBq to 1,000 MBq, from 50 MBq to 750 MBq, from 50 MBq to 500 MBq, from 50 MBq to 300 MBq, from 50 MBq to 200 MBq, or from 50 MBq to 100 MBq. In certain embodiments, the pharmaceutical composition has an activity from 100 MBq to 300 MBq. In certain embodiments, the pharmaceutical composition has an activity from 100 MBq to 200 MBq.

In certain embodiments, the pharmaceutical composition has an activity from 700 MBq to 1,500 MBq, e.g., from 700 MBq to 1,200 MBq, from 700 MBq to 1,000 MBq, from 750 MBq to 1,500 MBq, from 750 MBq to 1,200 MBq, from 750 MBq to 1,000 MBq, from 1,000 MBq to 1,500 MBq, from 1,200 MBq to 1,500 MBq, or from 1,200 MBq to 1,500 MBq.

In certain embodiments, the pharmaceutical composition has a radiotracer radiochemical purity of ≥90% at 12 hours after end of synthesis, e.g., ≥93%, ≥95%, ≥96%, ≥97%, ≥98%, or ≥99%.

In certain embodiments, the pharmaceutical composition has a radiotracer radiochemical purity from 90% to 99% at 12 hours after end of synthesis, e.g., from 90% to 98%, from 90% to 97%, from 90% to 95%, from 90% to 93%, from 93% to 99%, from 93% to 98%, from 93% to 97%, from 93% to 95%, from 95% to 99%, from 95% to 98%, from 95% to 97%, from 97% to 99%, or from 98% to 99%.

In certain embodiments, the pharmaceutical composition has a ⁶¹[Cu]Cu radionuclidic purity at end of synthesis of ≥95%, e.g., ≥96%, ≥97%, >98%, or ≥99%. In certain embodiments, the pharmaceutical composition has a ⁶¹[Cu]Cu radionuclidic purity at end of synthesis of ≥97%.

In certain embodiments, the pharmaceutical composition has a ⁶¹[Cu]Cu radionuclidic purity at end of synthesis of ≥98%, e.g., ≥99%, ≥99.5%, ≥99.6%, ≥99.7%, ≥99.8%, or ≥99.9%. In certain embodiments, the pharmaceutical composition has a ⁶¹[Cu]Cu radionuclidic purity at end of synthesis of ≥99.99%.

In certain embodiments, the pharmaceutical composition has a radiocobalt activity content at end of synthesis of ≤0.1%, e.g., ≤0.05%, ≤0.02%, ≤0.01%, ≤0.005% or ≤0.001%.

In certain embodiments, the pharmaceutical composition has at least one of a ⁵⁶[Co]Co specific activity or ⁵⁸[Co]Co specific activity at end of synthesis of ≤1,500 Bq/g, e.g., ≤1,200 Bq/g, ≤1,000 Bq/g, ≤800 Bq/g, ≤400 Bq/g, or ≤200 Bq/g.

In certain embodiments, the pharmaceutical composition has at least one of a ⁵⁶[Co]Co specific activity or ⁵⁸[Co]Co specific activity at end of synthesis from 200 Bq/g to 1,500 Bq/g, e.g., from 200 Bq/g to 1,200 Bq/g, from 200 Bq/g to 1,000 Bq/g, from 200 Bq/g to 800 Bq/g, from 200 to 600 Bq/g, or from 200 Bq/g to 400 Bq/g.

In certain embodiments, the pharmaceutical composition has a ⁵⁶[Co]Co specific activity of ≤100 Bq/g, e.g., ≤50 Bq/g, ≤25 Bq/g, ≤10 Bq/g, ≤8 Bq/g, ≤4 Bq/g, or ≤2 Bq/g.

In certain embodiments, the pharmaceutical composition has a ⁵⁶[Co]Co specific activity of from 1 Bq/g to 100 Bq/g, e.g., from 1 Bq/g to 50 Bq/g, from 1 Bq/g to 25 Bq/g, from 1 Bq/g to 10 Bq/g, from 1 Bq/g to 8 Bq/g, or from 1 Bq/g to 4 Bq/g.

In certain embodiments, the pharmaceutical composition has a ⁵⁸[Co]Co specific activity of ≤100 Bq/g, e.g., ≤50 Bq/g, ≤25 Bq/g, ≤10 Bq/g, ≤8 Bq/g, ≤4 Bq/g, or ≤2 Bq/g.

In certain embodiments, the pharmaceutical composition has a ⁵⁸[Co]Co specific activity of from 1 Bq/g to 100 Bq/g, e.g., from 1 Bq/g to 50 Bq/g, from 1 Bq/g to 25 Bq/g, from 1 Bq/g to 10 Bq/g, from 1 Bq/g to 8 Bq/g, or from 1 Bq/g to 4 Bq/g.

In certain embodiments, the pharmaceutical composition is characterized by a ^{110m}Ag specific activity≤0.1 Bq/g. In certain embodiments, the pharmaceutical composition is characterized by a ^{108m}Ag specific activity≤0.1 Bq/g. In certain embodiments, the pharmaceutical composition is characterized by a 10Cd specific activity≤0.1 Bq/g.

In certain embodiments, the pharmaceutical composition is characterized by at least two of: a ^{110m} Ag specific activity≤0.1 Bq/g, a ^{108m}Ag specific activity≤0.1 Bq/g, and a ¹⁰⁹Cd specific activity≤0.1 Bq/g. In certain embodiments, the pharmaceutical composition is characterized by a ^{110m}Ag specific activity of ≤0.1 Bq/g, a ^{108m}Ag specific activity≤0.1 Bq/g, and a ¹⁰⁹Cd specific activity≤0.1 Bq/g.

In certain embodiments, the sum of the specific activities of the radionuclidic impurities in the pharmaceutical composition is ≤8,000 Bq/g, e.g., ≤5,000 Bq/g, ≤3,000 Bq/g, ≤1200 Bq/g, ≤1,000 Bq/g, ≤800 Bq/g, or ≤500 Bq/g.

In certain embodiments, the pharmaceutical composition comprises Al in an amount≤1.2 ng/MBq. In certain embodiments, the pharmaceutical composition comprises Co in an amount≤0.2 ng/MBq. In certain embodiments, the pharmaceutical composition comprises Fe in an amount ≤1.7 ng/MBq. In certain embodiments, the pharmaceutical composition comprises Pb in an amount≤0.8 ng/MBq. In certain embodiments, the pharmaceutical composition comprises Zn in an amount≤0.8 ng/MBq. In certain embodiments, the pharmaceutical composition comprises one or more of: an amount of Al≤1.2 ng/MBq, an amount of Co≤0.2 ng/MBq, an amount of Fe≤1.7 ng/MBq, an amount of Pb≤0.8 ng/MBq, or an amount of Zn≤0.8 ng/MBq.

In certain embodiments, the pharmaceutical composition has an activity concentration≥10 MBq/mL, e.g., 10-25 MBq/mL, ≥20 MBq/mL, ≥30 MBq/mL, ≥40 MBq/mL, ≥50 MBq/mL, ≥60 MBq/mL, ≥70 MBq/mL, ≥80 MBq/mL, or ≥90 MBq/mL.

In certain embodiments, the pharmaceutical composition has an activity concentration from 10 MBq/mL to 100 MBq/mL, e.g., from 10 MBq/mL to 50 MBq/mL or from 40 MBq/mL to 90 MBq/mL.

In certain embodiments, the apparent molar activity of the radiotracer is ≥1 MBq/nmol, e.g., ≥10 MBq/nmol, ≥20 MBq/nmol, ≥30 MBq/nmol, or ≥50 MBq/nmol.

In certain embodiments, the apparent molar activity of the radiotracer is from 1 MBq/nmol to 50 MBq/nmol, e.g., from 1 MBq/nmol to 30 MBq/nmol, from 1 MBq/nmol to 20 MBq/nmol, from 1 MBq/nmol to 10 MBq/nmol, from 10 MBq/nmol to 50 MBq/nmol, from 10 MBq/nmol to 30 MBq/nmol, from 10 MBq/nmol to 20 MBq/nmol, from 20 MBq/nmol to 50 MBq/nmol, from 20 MBq/nmol to 40 MBq/nmol, or from 20 MBq/nmol to 30 MBq/nmol.

In certain embodiments, the molar activity of the radiotracer is ≥1 MBq/nmol, e.g., ≥10 MBq/nmol, ≥20 MBq/nmol, ≥30 MBq/nmol, or ≥50 MBq/nmol.

In certain embodiments, the molar activity of the radiotracer is from 1 MBq/nmol to 50 MBq/nmol, e.g., from 1 MBq/nmol to 30 MBq/nmol, from 1 MBq/nmol to 20 MBq/nmol, from 1 MBq/nmol to 10 MBq/nmol, from 10 MBq/nmol to 50 MBq/nmol, from 10 MBq/nmol to 30 MBq/nmol, from 10 MBq/nmol to 20 MBq/nmol, from 20 MBq/nmol to 50 MBq/nmol, from 20 MBq/nmol to 40 MBq/nmol, or from 20 MBq/nmol to 30 MBq/nmol.

In certain embodiments, the pH of the pharmaceutical composition is from 5 to 7, e.g., from 5 to 6, from 5.5 to 6.5, or from 6 to 7. In certain embodiments, the pH of the pharmaceutical composition is 5, 6, or 7+/−0.1.

### 4.2.2. Excipients

In certain embodiments, the pharmaceutical composition comprises one or more excipients.

In certain embodiments, the pharmaceutical composition comprises one or more radiolytic inhibitors. Exemplary radiolytic inhibitors include, but are not limited to, ascorbic acid, gentisic acid, citric acid, N-tert-butyl-α-phenylnitrone (PBN), polyvinylpyrrolidone (PVP), ethanol, DMSA, cysteine, vanillin, methionine, adenine, dobesilic acid, thymine, uracil, nicotinic acid, nicotinamide, salts of any of the foregoing, and combinations thereof.

In certain embodiments, the radiolytic inhibitor comprises ascorbic acid or a salt thereof. In certain embodiments, the radiolytic inhibitor comprises ascorbic acid. In certain embodiments, the radiolytic inhibitor comprises a salt of ascorbic acid, e.g., sodium ascorbate, calcium ascorbate, or potassium ascorbate.

In certain embodiments, the radiolytic inhibitor is present in the pharmaceutical composition in a concentration≥1 μg/mL, e.g., ≥2 μg/mL, ≥3 μg/mL, ≥5 μg/mL, ≥8 μg/mL, 10 μg/mL, or ≥15 μg/mL.

In certain embodiments, the radiolytic inhibitor is present in the pharmaceutical composition in a concentration from 1 μg/mL to 15 μg/mL, e.g., from 3 μg/mL to 15 μg/mL, from 3 μg/mL to 10 μg/mL, from 5 μg/mL to 10 μg/mL, from 5 μg/mL to 15 μg/mL, from 5 μg/mL to 10 μg/mL, from 8 μg/mL to 15 μg/mL, from 8 μg/mL to 10 μg/mL, or from 10 μg/mL to 15 μg/mL.

In certain embodiments, the pharmaceutical composition comprises sodium chloride. In certain embodiments, sodium chloride is present in the pharmaceutical composition a concentration from 0.5 mg/mL to 50 mg/mL, e.g., from 5 mg/mL to 10 mg/mL, from 10 mg/mL to 30 mg/mL, or from 20 mg/mL to 50 mg/mL. In certain embodiments, the pharmaceutical composition comprises sodium chloride in a concentration from 1 mg/mL to 10 mg/mL or 1 mg/mL to 5 mg/mL.

In certain embodiments, the pharmaceutical composition comprises hydrochloric acid. In certain embodiments, the hydrochloric acid is present in the pharmaceutical composition in a concentration from 0.1 mg/mL to 1.0 mg/mL.

In certain embodiments, the pharmaceutical composition comprises sodium acetate. In certain embodiments, the sodium acetate is present in the pharmaceutical composition in a concentration from 5 mg/mL to 50 mg/mL, e.g., from 10 mg/mL to 25 mg/mL or from 15 mg/mL to 20 mg/mL.

In certain embodiments, the hydrochloric acid reacts with sodium acetate forming acetic acid and sodium chloride.

In certain embodiments, the pharmaceutical composition comprises 1-100 μg of the radiotracer, 3 μg/mL to 15 μg/mL of a radiolytic inhibitor (e.g., ascorbic acid or a salt thereof), and 1 mg/mL to 10 mg/mL sodium chloride.

In certain embodiments, the pharmaceutical composition comprises 10-100 μg of the radiotracer, 3 μg/mL to 15 μg/mL of a radiolytic inhibitor (e.g., ascorbic acid or a salt thereof), and 1 mg/mL to 10 mg/mL sodium chloride.

In certain embodiments, the pharmaceutical composition comprises 10-100 μg of the radiotracer, 3 μg/mL to 15 μg/mL of a radiolytic inhibitor (e.g., ascorbic acid or a salt thereof), 1 mg/mL to 10 mg/mL sodium chloride, 0.1 mg/mL to 1.0 mg/mL hydrochloric acid, and 10 mg/mL to 25 mg/mL sodium acetate.

### 4.3. Methods of Imaging

In one aspect, the present disclosure provides methods for imaging subjects suspected of having or diagnosed with a PMSA-expressing tumor comprising administering an effective amount of a pharmaceutical composition of the present disclosure and generating one or more radiographic images of the subject.

In another aspect, the present disclosure provides methods for determining a subject's response to a cancer treatment over time, e.g., a subject previously diagnosed with a PSMA-expressing tumor. In certain embodiments, the method comprises:
(a) administering an effective amount of a pharmaceutical composition of the present disclosure to a subject diagnosed with a PSMA-expressing tumor at an earlier time point and at a later time point;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

In certain embodiments, the subject is a mammal. In certain embodiments, the subject is a human. In certain embodiments, the subject is a human≥18 years of age.

In certain embodiments, the subject has been diagnosed with one or more PSMA-expressing tumors. In certain embodiments, the subject is suspected of having, but not diagnosed with, one or more PSMA-expressing tumors.

Exemplary PSMA-expressing tumors include, but are not limited to, a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, and combinations thereof. In certain embodiments, the PSMA-expressing tumor is a prostate tumor. In certain embodiments, the PSMA-expressing tumor is a metastasized prostate tumor.

In certain embodiments, the PSMA-expressing tumor in the subject was detected by PET/CT with [⁶⁸Ga]Ga-PSMA-11 (ILLUCCIX®, LOCAMETX®), [¹⁸F]F-Piflufolastat (PYLARIFY®), or [¹⁸F]F-Flotufolastat (POSLUMA®).

In certain embodiments, the subject has been diagnosed with a prostate cancer. In certain embodiments, the subject has been diagnosed with recurrent prostate cancer. In certain embodiments, the subject has been diagnosed with castration-resistant prostate cancer.

In certain embodiments, the subject has a biopsy proven prostate adenocarcinoma.

In certain embodiments, the subject is receiving cancer treatment at the time of administering the pharmaceutical composition or has received cancer treatment in the last five years prior to administering the pharmaceutical composition, e.g., at least 1 week, at least 1 month, at least 3 months, at least 6 months, at least 1 year, at least 2 years, at least 3 years, at least 4 years, or 5 years.

In certain embodiments, the cancer treatment comprises surgery, radiation therapy, ablative therapy, hormone therapy, immunotherapy, chemotherapy, gene therapy, cryotherapy, or a combination thereof.

In certain embodiments, the cancer treatment comprises surgery. In certain embodiments, the subject has undergone more than one surgery.

In certain embodiments, the surgery comprises prostatectomy (radical or retropubic), transurethral resection of the prostate (TURP), orchiectomy (castration), or a combination thereof.

In certain embodiments, the cancer treatment comprises radiation therapy. In certain embodiments, radiation therapy comprises administration of external beam radiation therapy (ERT), e.g., three-dimensional conformal radiation therapy (3D-CRT), intensity modulated radiation therapy (IMRT), stereostatic body radiation therapy (SBRT), MRI-guided radiation therapy, proton beam radiation therapy, or brachytherapy (e.g., permanent brachytherapy and temporary brachytherapy).

In certain embodiments, radiation therapy comprises administration of a radiopharmaceutical, e.g., ¹⁷⁷[Lu]-PSMA-vipivotide tetraxetan (PLUVICTO®), Strontium-89 (METASTRON®), Samarium-153 lexidronam (QUADRAMET®), Radium-223 (XOFIGO®), ¹⁷⁷[Lu]Lu-PSMA-I&T, ²²⁵[Ac]Ac-J591, ¹⁷⁷[Lu]Lu-J591, TLX591, ²²⁵[Ac]Ac-TLX592, ²²⁷[Th]Th-PSMA-TTC, I-131-1095, ¹⁷⁷[Lu]Lu-DOTA-N3-CTT1403, ²²⁵[Ac]Ac-PSMA-617, ¹⁷⁷[Lu]Lu-PSMA-R2, ⁶⁷[Cu]Cu-PSMA), or a combination thereof.

In certain embodiments, the cancer treatment comprises ablative therapy. In certain embodiments, ablative therapy comprises, e.g., administration of cryotherapy, high-intensity focused ultrasound (HIFU), photodynamic therapy (PDT), focal laser ablation (FLA), or a combination thereof.

In certain embodiments, the cancer treatment comprises hormone therapy. In certain embodiments, hormone therapy comprises administration of Luteinizing hormone-releasing hormone (LHRH) agonists (e.g., Leuprolide, LUPRON® and ELIGARD®; leuprolide mesylate, CAMCEVI®; goserelin, ZOLADEX®; triptorelin, TRELSTAR®), LHRH antagonists (e.g., degarelix, FIRMAGON®; and relugolix, ORGOVYX®), abiraterone (ZYTIGA®), ketoconazole (NIZORAL®), flutamide (EULEXIN®), bicalutamide (CASODEX®), nilutamide (NILANDRON®), enzalutamide (XTANDI®), apalutamide (ERLEADA®), darolutamide (NUBEQA®), or a combination thereof.

In certain embodiments, the cancer treatment comprises immunotherapy. In certain embodiments, immunotherapy comprises administration of a vaccine (e.g., Sipuleucel-T, PROVENGE®), an immune checkpoint inhibitor (e.g., pembrolizumab, KEYTRUDA®; dostarlimab, JEMPERLI®; ipilimumab, YERVOY®; nivolumab, OPDIVO®), or a combination thereof.

In certain embodiments, the cancer treatment comprises chemotherapy. In certain embodiments, chemotherapy comprises administration of docetaxel (TAXOTERE®), cabazitaxel (JEVTANA®), or a combination thereof.

In certain embodiments, the subject is not co-administered a diuretic with the pharmaceutical composition of the present disclosure. Exemplary diuretics include, e.g., furosemide, L-lysine, spironolactone, bumetanide, hydrochlorothiazide, metolazone, and torsemide.

In certain embodiments, the pharmaceutical composition is administered intravenously. In certain embodiments, administration occurs as a single bolus infusion. In certain embodiments, administration occurs over two or more separate infusions.

In certain embodiments, the pharmaceutical composition is administered over a period of time of at least 5 seconds, e.g., at least 10 seconds, at least 15 seconds, at least 30 seconds, at least 1 minute, at least 5 minutes, or at least 10 minutes.

In certain embodiments, the pharmaceutical composition is administered over a period of time from 5 seconds to 10 minutes, e.g., from 5 seconds to 5 minutes, from 5 seconds to 1 minute, from 5 seconds to 30 seconds, from 5 seconds to 20 seconds, from 10 seconds to 10 minutes, from 10 seconds to 5 minutes, from 10 seconds to 1 minute, from 10 seconds to 30 seconds, from 30 seconds to 10 minutes, from 30 seconds to 5 minutes, from 30 seconds to 1 minute, from 1 minute to 10 minutes, from 1 minute to 5 minutes, or from 5 minutes to 10 minutes.

In certain embodiments, the effective dose per MBq of administered pharmaceutical composition is ≤20 μSv/MBq, e.g., ≤15 μSv/MBq, ≤10 μSv/MBq, ≤7.5 μSv/MBq, ≤5 μSv/MBq, or ≤3 μSv/MBq.

In certain embodiments, the effective dose per MBq of administered pharmaceutical composition is from 3 μSv/MBq to 20 μSv/MBq, e.g., from 3 μSv/MBq to 15 μSv/MBq, from 3 μSv/MBq to 10 μSv/MBq, from 3 μSv/MBq to 7.5 μSv/MBq, from 3 μSv/MBq to 5 μSv/MBq, from 5 μSv/MBq to 20 μSv/MBq, from 5 μSv/MBq to 15 μSv/MBq, from 5 μSv/MBq to 10 μSv/MBq, from 5 μSv/MBq to 7.5 μSv/MBq, from 7.5 μSv/MBq to 20 μSv/MBq, from 7.5 μSv/MBq to 15 μSv/MBq, from 7.5 μSv/MBq to 10 μSv/MBq, from 10 μSv/MBq to 20 μSv/MBq, from 10 μSv/MBq to 15 μSv/MBq, or from 15 μSv/MBq to 20 μSv/MBq.

In certain embodiments, the effective dose per MBq of administered pharmaceutical composition is from 5 μSv/MBq to 20 μSv/MBq, e.g., from 5 μSv/MBq to 10 μSv/MBq, from 5 μSv/MBq to 15 μSv/MBq, from 10 μSv/MBq to 20 μSv/MBq, or from 15 μSv/MBq to 20 μSv/MBq.

In certain embodiments, after administering to the subject a pharmaceutical composition disclosed herein having an activity of 100-300 MBq (e.g., 100-200 MBq, 100-150 MBq, 150-300 MBq, 150-200 MBq, 200-300 MBq, or 250-300 MBq), the absorbed dose in the kidneys is 1 Gy or less, e.g., 0.5 Gy or less, 0.3 Gy or less, or 0.2 Gy or less.

The methods of the present disclosure include a step of generating one or more radiographic images of the subject. The images can be of a region, body part, or organ of the subject. In certain embodiments, the one or more images are generated of the skull to mid-thigh of the subject. In certain embodiments, the one or more radiographic images are generated of the lacrimal glands, salivary glands, liver, spleen, kidneys, ureters, bladder, lungs, proximal small bowel, prostate, small intestine, adrenal glands, lymph nodes, or combinations thereof.

The radiographic images can be generated using positron emission tomography (PET), PET-computer tomography (PET-CT), or single-photon emission computerized tomography (SPECT). In certain embodiments, the one or more radiographic images are generated using PET. In certain embodiments, the one or more radiographic images are generated using PET-CT. In certain embodiments, the one or more radiographic images are generated using SPECT.

In certain embodiments, the one or more radiographic images of the subject are generated dynamically, i.e., concurrently with administration of the pharmaceutical composition.

In certain embodiments, the one or more radiographic images are generated after administration of the pharmaceutical composition. In certain embodiments, the one or more radiographic images are generated at least 30 minutes after administration of the pharmaceutical composition, e.g., at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, or at least 24 hours after administration of the pharmaceutical composition.

In certain embodiments, the one or more radiographic images are generated from 30 minutes to 24 hours after administration of the pharmaceutical composition, e.g., from 30 minutes to 18 hours, from 30 minutes to 10 hours, from 30 minutes to 5 hours, from 30 minutes to 3 hours, from 30 minutes to 1 hour, from 1 hour to 24 hours, from 1 hour to 18 hours, from 1 hour to 10 hours, from 1 hour to 5 hours, from 1 hour to 3 hours, from 3 hours to 24 hours, from 3 hours to 18 hours, from 3 hours to 10 hours, from 3 hours to 5 hours, from 5 hours to 24 hours, from 5 hours to 18 hours, from 5 hours to 10 hours, from 10 hours to 24 hours, from 10 hours to 18 hours, or from 18 hours to 24 hours after administration of the pharmaceutical composition.

In certain embodiments, the one or more radiographic images are generated 1 hour after administration of the pharmaceutical composition.

In certain embodiments, the method further comprises determining one or more of the following from the radiographic images: maximum standardized uptake volume (SUVmax), tumor detection rate (TDR), differential tumor detection rate (DDR), signal-to-noise ratio (SNR=SUVmax in tumor volume of interest (VOI)), tumor-to-background ratio(s) (TBR), sensitivity, and positivity predictive value (PPV).

In certain embodiments, the methods of the present disclosure identify the same number of PSMA-positive malignant lesions than when the method is carried out with a standard of care pharmaceutical composition comprising a radiotracer, e.g., ¹⁸[F]-Piflufolastat (PYLARIFY®), ¹⁸F-PSMA1007, ¹⁸F-rhPSMA-7.3 (POSLUMA®), and ⁶⁸Ga]Ga-PSMA-11 (ILLUCCIX®, LOCAMETX®).

In certain embodiments, the methods of the present disclosure identify more PSMA-positive malignant lesions than when the method is carried out with a standard of care pharmaceutical composition comprising a radiotracer, e.g., ¹⁸[F]-Piflufolastat (PYLARIFY®), ¹⁸F-PSMA1007, ¹⁸F-rhPSMA-7.3 (POSLUMA®), and ⁶⁸Ga]Ga-PSMA-11 (ILLUCCIX®, LOCAMETX®).

In certain embodiments, following generating one or more images, localization (absolute) and amount of localization of the radionuclide in the one or more radiographic images of the subject can be determined, e.g., by medical personnel.

Based on the localization of the radionuclide, one or more of the following can occur: staging or restaging the subject's cancer, determining the subject's cancer treatment protocol, and determining the subject's response to cancer treatment.

In certain embodiments, reading of the one or more radiographic images generated by the methods described herein enables staging the subject's cancer. In certain embodiments, reading of the one or more radiographic images generated by the methods described herein enables restaging the subject's cancer. A number of staging methods are known in the art, e.g., the “TNM system,”, where “T” is the primary tumor (T category), “N” is lymph nodes (N category” and “M” is metastasis (M category). In certain embodiments, a cancer's grade is included in the stage. Once the values for T, N, and M (and any other factors that affect stage) have been determined, they are combined to assign an overall stage. For most cancers, the stage is a Roman numeral from I (1) to IV (4). Stage I cancers are less advanced and often have a better prognosis (outlook). Higher stage cancers typically have spread farther (or have other concerning features) and might require more intense (or different kinds of) treatment. Sometimes stages are subdivided as well, using capital letters (for example, stage III might be subdivided into stages IIIA and IIIB). Some cancers also have a stage 0, which is often called carcinoma in situ. This means the cancer is still only in the layer of cells where it first started, and it has not spread any farther.

In certain embodiments, reading of the one or more radiographic images generated by the methods described herein enables determination of the subject's cancer treatment protocol, e.g., surgery, radiation therapy, ablative therapy, hormone therapy, immunotherapy, chemotherapy, gene therapy, cryotherapy, or a combination thereof.

In certain embodiments, reading of the one or more radiographic images generated by the methods described herein enables determination of the subject's response to cancer treatment, e.g., responding to treatment or not responding to treatment. In such embodiments, the pharmaceutical composition is administered to the subject at an earlier time point and at a later time point, and one or more images are generated at both the earlier time point and the later time point. The images generated at the earlier time point and later time point are compared. Subjects responding to treatment have decreased radiotracer localization at the later imaging time point compared to the earlier imaging timepoint.

In certain embodiments, the earlier time point is before the subject begins cancer treatment. In certain embodiments, the earlier time point is at least 1 day before the subject begins cancer treatment, e.g., at least 1 week, at least 2 weeks, at least 3 weeks, or at least 1 month before the subject begins cancer treatment.

In certain embodiments, the earlier time point is after the subject begins cancer treatment. In certain embodiments, the earlier time point is at least 1 week after the subject begins cancer treatment, e.g., at least 2 weeks, at least 1 month, at least 2 months, at least 3 months, at least 6 months, at least 9 months, or at least 12 months after the subject begins cancer treatment.

In certain embodiments, the later time point is at least 1 day after the earlier time point, e.g., at least 2 days, at least 3 days, at least 4 days, at least 5 days, at least 6 days, at least 1 week, at least 2 weeks, at least 3 weeks, at least 4 weeks, at least 2 months, at least 3 months, at least 6 months, at least 9 months, or at least 1 year after the earlier time point.

In certain embodiments, the later time point is after the subject finishes cancer treatment. In certain embodiments, the later time point is at least 1 week after the subject finishes cancer treatment, e.g., at least 2 weeks, at least 1 month, at least 2 months, at least 3 months, at least 6 months, at least 9 months, or at least 12 months after the subject finishes cancer treatment.

In certain embodiments, the earlier time point is before the subject begins cancer treatment and the later time point is after the subject finishes cancer treatment (i.e., to determine the effectiveness of a previous treatment). In certain embodiments, the earlier time point is before the subject begins cancer treatment and the later time point is after the subject begins cancer treatment (i.e., to determine the effectiveness of the ongoing treatment).

In certain embodiments, following generation of the one or more radiographic images, the subject is then treated with a second pharmaceutical composition comprising a radiopharmaceutical (i.e., theranostics). In certain embodiments, the radiopharmaceutical is selected from ⁶⁷[Cu]Cu-NODAGA-PSMA I&T, ¹⁷⁷[Lu]Lu-DOTA-PSMA I&T, ¹⁷⁷[Yb]Yb-DOTA-PSMA I&T, ²²⁵[Ac]Ac-DOTA-PSMA I&T, ²¹²[Pb]Pb-DOTA-PSMA I&T, ¹⁷⁷[Lu]Lu-PSMA-vipivotide tetraxetan, ²²⁵[Ac]Ac-J591, ¹⁷⁷[Lu]Lu-J591, TLX591, ²²⁵[Ac]Ac-TLX592, ²²⁷[Th]Th-PSMA-TTC, I-131-1095, ¹⁷⁷[Lu]Lu-DOTA-N3-CTT1403, ²²⁵[Ac]Ac-PSMA-617, ¹⁷⁷[Lu]Lu-PSMA-R2, and ⁶⁷[Cu]Cu-PSMA.

In another aspect, the present disclosure provides theranostic methods comprising administering to a subject an effective amount of a first composition corresponding to a pharmaceutical composition described hereinabove, generating one or more images of the subject, and administering to the subject an effective amount of a second pharmaceutical composition comprising a therapeutic radiopharmaceutical. Administration of the first composition corresponding to the pharmaceutical composition described herein and generation of the one or more images is carried out as set forth above. In certain embodiments, radiopharmaceutical of the second composition is selected from ⁶⁷[Cu]Cu-NODAGA-PSMA I&T, ¹⁷⁷[Lu]Lu-DOTA-PSMA I&T, ¹⁷⁷[Yb]Yb-DOTA-PSMA I&T, ²²⁵[Ac]Ac-DOTA-PSMA I&T, ²¹²[Pb]Pb-DOTA-PSMA I&T, ¹⁷⁷[Lu]Lu-PSMA-vipivotide tetraxetan, ²²⁵[Ac]Ac-J591, ¹⁷⁷[Lu]Lu-J591, TLX591, ²²⁵[Ac]Ac-TLX592, ²²⁷[Th]Th-PSMA-TTC, I-131-1095, ¹⁷⁷[Lu]Lu-DOTA-N3-CTT1403, ²²⁵[Ac]Ac-PSMA-617, ¹⁷⁷[Lu]Lu-PSMA-R2, and ⁶⁷[Cu]Cu-PSMA.

### 5. EXAMPLES

### 5.1. Example 1: [⁶¹Cu]CuCl₂ Production

The production of the radionuclide was performed by irradiating a solid target of highly pure ⁶¹Ni (purity>99.42%) plated on a niobium coin backing with a proton beam.

Due to the relatively short half-lives (t_{1/2} ⁶⁸Ga=68 min; ¹⁸F=110 min) and physical properties of the radionuclides, the key challenges in the PET tracer industry remain a) the imaging quality, b) reliability of supply and distribution of the radiopharmaceutical at low cost and c) low radiation burden to the patient. The distinctive advantage of using ⁶¹Cu as a positron emitter, e.g., in a PET tracer, will not only ensure a) good imaging quality due to its physical properties (low mean positron energy) but also the possibility of delayed imaging, expected to improve the diagnostic sensitivity due to the washout of radioactivity from the background, thus improved image contrast, b) a large distribution radius due to its relatively long half-life (t_{1/2} ⁶¹Cu=205.5 min) while c) still keeping the radiation burden to the patient at a minimum. Provided herewith is an enabling description of new processes to produce highly pure ⁶¹Cu, in the form of [⁶¹Cu]CuCl₂, to be used in radiopharmaceutical applications, e.g., as a positron emitter in a PET tracer, in high activity concentration and volumes.

Trace metals and cold copper compete with ⁶¹Cu to bind a chelator (for example, NODAGA) in this order: cold Cu(II) (i.e., stable isotopes)>Zn(II)>Fe(III)>Sn(IV)>Ti(IV)>Al(III.). The competition from these trace metals and cold copper decreases the tracer's radiolabeling yield and radiochemical purity significantly. Frequent sources of trace metals are the raw nickel metal powder itself, especially isotopically enriched nickel, reagents, and any metals in instruments used, such as iron. The purification process (ion-exchange columns) removes much of the trace metals except for cold (of particular relevance are stable isotopes ⁶⁹Cu and ⁶⁵Cu), which passes through into the product fraction by being the same element as the desired ⁶¹Cu. One way of preventing cold copper contamination and the associated reduction in chemical purity is to pass the dissolved nickel raw material (stable isotopes) through the process and separate the cold copper from the nickel before plating (see FIG. 8 for ICP-MS analysis). Table 1 displays the chemical purity of the [⁶¹Cu]CuCl₂ by either bombardment of ^{nat}Ni or ⁶¹Ni on a niobium backing and the resulting impurity profile.

TABLE 1
Chemical Purity of ⁶¹Cu transmuted from ^{nat}Ni vs. ⁶¹Ni.

| | ng/MBq | |
|---|---|---|
| | Cu-61 from nat-Ni | Cu-61 from Ni-61 |
| Aluminum (Al) | 1.1 | 0.3 |
| Cobalt (Co) | N/D | 0.2 |
| Copper (Cu) | 0.3 | 0.6 |
| Iron (Fe) | 1.6 | 1.5 |
| Lead (Pb) | 0.4 | 0.7 |
| Nickel (Ni) | 3.4 | 0.1 |
| Zinc (Zn) | 0.7 | 0.1 |

### 5.1.1. Preparation of Plating Solution

### 5.1.1.1 Preparation of Buffer Solution

Ammonium Chloride (4.6 g, Aldrich: 326372, Trace Select) was weighed into a clean (no metal) Falcon Tube (50 mL), and the previously cleaned magnetic stirring bar was added. 6 mL of Trace Select water (Honeywell 95305) was added in one aliquot to flush walls of the Falcon in case any salt sticks to the Falcon tube walls. 1 mL of ammonium hydroxide 28% (Sigma 338818) was added with a 1000 μL pipette with a respective pipette tip, 8× times. The lid of the Falcon was closed, and the Falcon is, in turns, vortexed (1-2 minutes) (immersion in an ultra-sonic bath was a possible alternative for 1-2 minutes) and shaken, until all salt was dissolved. The Falcon tube can also be warmed (e.g., by rolling between hands) to improve solubility, temperature (e.g., around 23° C., preferably between 23-25° C.). After complete dissolution of the salt, the pH acceptance criteria, pH range 9.28-9.62, was verified by pH measurement of the solution at RT, e.g., with and electronic pH meter. The Falcon tube was closed with parafilm and stored at room temperature. Prior to use, any solid salt formation was redissolved.

### 5.1.1.2 Preparation of Nickel Nitrate Plating Solution

A 50 mL glass beaker was washed with nitric acid (Trace Select) followed by water (Trace Select). In a fume hood, the beaker was dried by placing it on a heating plate set to 150° C. To the beaker was added 210 μg of natural (isotopic distribution) nickel (powder, Sigma-Aldrich<50 μm, 99.7% trace metals basis, essentially free from any impurities, except iron. The copper impurity amounts to <0.3 ppm.) were weighed into the beaker and 4 mL of 65% nitric acid were added using a pipette. The beaker was placed back on the active heating plate and the stirring was set to 300 rpm. Ensure the ventilation of the fume hood was functioning properly (evolution of NO₂). During the dissolution, the solution turned green. The solution was reduced by evaporation to a volume of ≈600 μL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. Buffer solution (4 mL), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

The following are example lots of ⁶⁰Ni and ⁶¹Ni (certificate as provided by Isoflex, USA, March 2018):

TABLE 2

| Isotope | ⁶¹Ni | | | | | | | | | | |
|---|---|---|---|---|---|---|---|---|---|---|---|
| Enrichment | 86.20% | | | | | | | | | | |
| Form | Metal | | | | | | | | | | |
| | ingot/powder | | | | | | | | | | |
| Certificate | 6275 | | | | | | | | | | |
| | Isotope | Ni-58 | Ni-60 | Ni-61 | Ni-62 | Ni-64 | | | | | |
| Isotopic | Content (%) | 1.17 | 0.8 | 86.2 | 11.7 | 0.14 | | | | | |
| distribution | | | | | | | | | | | |
| | Element | Al | Bi | Ca | Cd | Co | Cr | Cu | Fe | K | μg |
| Chemical | Content | 10 | <10 | 20 | 10 | <10 | <10 | 20 | 40 | <10 | <50 |
| admixtures | (ppm) | | | | | | | | | | |
| | Element | Mo | Mn | Na | Pb | Si | Sn | Zn | | | |
| | Content | <8 | <50 | <10 | <10 | 20 | 30 | 50 | | | |
| | (ppm) | | | | | | | | | | |

TABLE 3

| Isotope | ⁶¹Ni | | | | | | | | | | |
|---|---|---|---|---|---|---|---|---|---|---|---|
| Enrichment | 99.39% | | | | | | | | | | |
| Form | Metal | | | | | | | | | | |
| | powder | | | | | | | | | | |
| Certificate | TBD/not | | | | | | | | | | |
| | specified | | | | | | | | | | |
| | Isotope | Ni-58 | Ni-60 | Ni-61 | Ni-62 | Ni-64 | | | | | |
| Isotopic | Content | 0.01 | 0.29 | 99.39 | 0.29 | 0.02 | | | | | |
| distribution | (%) | | | | | | | | | | |
| | Element | Al | Co | Cr | Cu | Fe | μg | Mn | Pb | Si | Ti |
| Chemical | Content | 12 | <10 | <10 | 14 | <10 | <10 | <10 | <10 | <10 | <10 |
| admixtures | (ppm) | | | | | | | | | | |
| | Element | Zn | C | S | | | | | | | |
| | Content | <10 | 157 | <10 | | | | | | | |
| | (ppm) | | | | | | | | | | |

TABLE 4

| Isotope | ⁶⁰Ni | | | | | | | | | | |
|---|---|---|---|---|---|---|---|---|---|---|---|
| Enrichment | 99.31% | | | | | | | | | | |
| Form | metal | | | | | | | | | | |
| | powder | | | | | | | | | | |
| Certificate | TBD/not | | | | | | | | | | |
| | specified | | | | | | | | | | |
| | Isotope | Ni-58 | Ni-60 | Ni-61 | Ni-62 | Ni-64 | | | | | |
| Isotopic | Content | 0.21 | 99.31 | 0.46 | 0.015 | 0.005 | | | | | |
| distribution | (%) | | | | | | | | | | |
| | Element | Al | Co | Cr | Cu | Fe | μg | Mn | Pb | Si | Ti |
| Chemical | Content | <10 | 70 | 20 | 25 | <10 | <10 | <10 | <10 | 15 | <10 |
| admixtures | (ppm) | | | | | | | | | | |
| | Element | Zn | C | S | P | | | | | | |
| | Content | 15 | 114 | 20 | 30 | | | | | | |
| | (ppm) | | | | | | | | | | |

The samples of natural nickel from Sigma-Aldrich were essentially free from any impurities, except iron. The copper impurity amounts to <0.3 ppm. Additional suitable sources of natural Ni include:
Nickel powder, <50 μm, 99.7% trace metals basis
Nickel rod, diam. 6.35 mm, =99.99% trace metals basis
Nickel foil, thickness 0.5 mm, 99.98% trace metals

### 5.1.2. Electroplating the Backing Surface

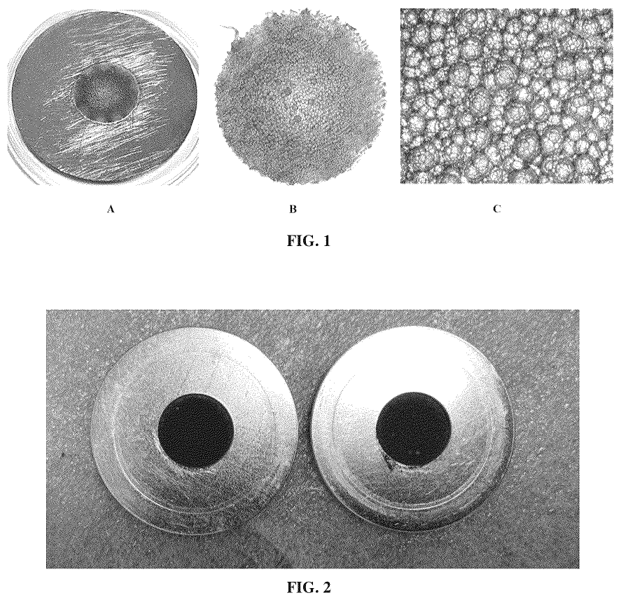

A disc shaped niobium backing was obtained from high purity Nb as described herein and (28 mm×1.0 mm) was cleaned with ethanol (high-purity) and inserted in a Comecer Electroplating Unit V21204. A platinum wire anode was positioned so that the distance relative to the coin surface was between about 1 and 3 mm, adjusted by a polymer spacer. The coin mass was determined to be 5.25 grams. Niobium backing (22 mm×1.0 mm weighs 3.3 g). The plating solution was charged to the electrolyte container and attached to the apparatus. The voltage was set to 4.5V. The current reading after 5 min stabilization was 180 μA. The duty cycle for pump was set to 45%. The plating liquid turned from blue to transparent, slow decrease of current to 160 μA was observed over the period of 120 minutes. The plating process was stopped. The coin was taken out of the electrolytic cell and its weight was measured. The coin also underwent microscopic evaluation, FIGS. 1 and 2 using a DINOLite digital microscope to observe the crystal structure and homogeneity of the surface. The coin (FIG. 2) was stored in a metal-free Falcon tube under a nitrogen atmosphere.

### 5.1.3. Results of the Electroplating

Upon completion of electroplating, the coin underwent a microscopic evaluation using a DINOLite digital microscope to observe the crystal structure and homogeneity of the surface. As can be seen in FIG. 1 (panels A-C), a homogenous target coating having durable adhesion was obtained, see also FIG. 2.

5.1.4. General Guidelines for High-purity [⁶¹Cu]Cl₂ Production

The purpose of this example was to enable the bulk production of ⁶¹Cu from the deuteron irradiation of natural nickel and/or enriched ⁶⁰Ni. This effort was a proof of concept, and, therefore, there were no benchmarked specifications for ⁶¹Cu. However, we optimized target performance, target geometry/material use, irradiation parameters, and chemical processing methods to produce [⁶¹Cu]CuCl₂ following enriched ⁶⁰Ni irradiation, or, scaled accordingly for ^{nat}Ni irradiation. There were no pharmacopoeia specifications for radio-copper explicitly, however, test QC methods include assessment of radionuclidic purity and apparent molar activity (to demonstrate usability of the extracted [⁶¹Cu]CuCl₂).

This example considers use of two different types of targets, natural nickel (^{nat}Ni) targets and highly enriched Nickel-60 (⁶⁰Ni) targets both of which were suitable for deuteron bombardment. However, ^{nat}Ni was cheap and available in high-purity while ⁶⁰Ni was still costly and required efficiency measures. If even higher yields were desired, target preparation efforts may be directly translated into the proton-based ⁶¹Ni(p,n) ⁶¹Cu route, however, given the cost of enriched ⁶¹Ni (c.a. $25 USD/μg), such an approach imposes the need for target recycling.

The set of guidelines below enable all types of targets in the production of ⁶¹Cu, including the production of high-purity [⁶¹Cu]CuCl₂ from the Nb coins with a Zn or Ni (any isotopic enrichment) coating electroplated thereon as provided herein. Specific details are also provided for deuteron, and proton irradiations, respectively. This protocol was followed to generate the ⁶¹Cu-compositions evaluated in the following examples.

| Target Backing | Flat coin - disc-shaped. The dimensions of the target backing form are: | |
|---|---|---|
| Geometry | Coin backing: | |
| | Diameter Ø = 20 − 30 ± 0.1 mm | |
| | Thickness H = 1.5 mm | |
| | Target Ni layer or coating | |
| | a. Diameter 13 mm (deuteron) or 10 mm (proton) | |
| | b. Mass 70-100 mg, e.g., around 100 ± 40 mg (deuteron) or | |
| | around 50 ± 20 mg (proton) | |
| | c. Thickness (H) full density (d = 8.9) | |
| | i. Hₘᵢₙ = 0.1 mm; Hₘₐₓ = 0.14 mm corresponding to 70- | |
| | 100 mg deposited | |
| | Tolerances/finishes unless otherwise stated are as follows: | |
| | Surface finish: Ra 1.6 | |
| | General tolerance: ISO 2768-m | |
| | Sharp edges and corners according to ISO 13 715 | |
| Target Backing | Optional - Surface treated with abrasion by pink corundum grindstone - | |
| Surface | free of impurities | |
| Target Backing | Niobium foil, 99.8% (metals basis), 1.0 mm (0.04 in) thick, annealed, | |
| Material | Stock No.: 10257 | |
| | Lot No.: C15P07 | |
| | Element | ppm |
| | Carbon | 24 |
| | Hydrogen | 1 |
| | Molybdenum | 2 |
| | Nickel | 4 |
| | Silicon | 1 |
| | Titanium | 2 |
| | Zirconium | 3 |
| | Iron | 1 |
| | Hafnium | 2 |
| | Nitrogen | 14 |
| | Oxygen | 56 |
| | Tantalum | 785 |
| | Tungsten | 4 |
| Target Backing | Niobium 99.9% typical certificate of analysis results, Goodfellows | |
| Material | Product nr. 931-627-20 | |
| | Element | ppm |
| | B | <10 ppm |
| | Ni | <5 ppm |
| | O | 100 ppm |
| | Si | 100 ppm |
| | Zr | <10 ppm |
| | Ta | 500 ppm |
| | H | <10 ppm |
| | W | <100 ppm |
| | C | 25 ppm |
| | N | 20 ppm |
| | Fe | 30 ppm |
| | Cu | <5 ppm |
| | Mo | 10 ppm |
| | Ti | <10 ppm |

| | |
|---|---|
| Transfer system | As the target can be automatically transferred to/from the cyclotron by |
| combatibility | means of a pneumatic target transfer system, it was critical that the |
| | deposited Ni was robust to direct air flow and abrupt mechanical |
| | movements. |
| | In certain embodiments, the target coating remains adhered to the backing |
| | during pneumatic transfer both to and from the cyclotron. Such a pneumatic |
| | system was typically fed by a compressed air connection of ~6-7 bar, and at |
| | minimum, 360 SLPM flow. Such a system was “push-push”, and therefore, |
| | compressed air was typically blown on both the front and rear sides of the |
| | coin, respectively, depending on the direction of transfer. The coin will also |
| | come to an abrupt stop as it reaches the target station or hot cell. |
| | In certain embodiments, suitable tests that indicate target durability include |
| | the following, whereby the total mass loss for all tests combined should be |
| | negligible (e.g. <1 μg): Visual inspection, gentle knocking/tapping on a |
| | countertop on top of white paper to check for loosening of target coating |
| | grains, gently rubbing an acid-washed Teflon spatula against the deposited |
| | target coating and checking for loosening of target coating grains, and/or |
| | placing and gently pressing down on a piece of Scotch tape against the |
| | target coating. |
| | If there was access to the cyclotron apparatus, it was recommended to |
| | transfer the coin back/forth multiple times and ensure target coating |
| | stability (i.e., no mass loss). Such a test may be performed with a degrader |
| | in place. |
| Method of | Electrodeposition from bath with a significantly high pH (e.g., 9.9-10.8) |
| Production | |
| Target Metal | To withstand the deposited beam power, the target metal was preferably |
| Form | metallic nickel (not, e.g., nickel oxide). |
| | Depending on the means of target preparation (e.g. electroplating), the raw |
| | nickel starting material need not necessarily be metallic. However, methods |
| | used for preparing ^{nat}Ni targets should ultimately be directly translatable to |
| | preparation of ⁶⁰Ni or ⁶¹Ni targets. At present, it was understood that |
| | enriched Ni was typically in the form of a salt. |
| Target Additives | The use of binders must not necessary be avoided if they are absent of the |
| | final metallic coin and if an assessment on a case-by-case basis to |
| | understand potential impact to product quality has been done (e.g. ICP-MS |
| | on the binder material. |
| | Any reagents used for target preparation (e.g. electroplating reagents) must |
| | be of the highest quality, in particular, with regards to trace metals. |
| Metal Content | Preferably, the highest grades of reagents should be used, to avoid trace |
| | metals contamination of the target coating, as more than a tenth of a |
| | microgram per 100 μg of target metal (that is, 1 ppm of the target metal) is |
| | already a significant contamination that may render the coin unusable for |
| | production of high-purity radionuclides. In the case of the production of |
| | radiocopper it is not accepted to add more than 0.1 ppm of cold Cu as this |
| | would reduce the purity of the prepared radionuclide composition. |
| | Preferably, max level of impurities allowed to be added by the process to |
| | the initial nickel: |
| | Copper (Cu): 0.1 ppm |
| | High affinity metals (Ga, Lu, Pb, Y): 0.1 ppm |
| | Zinc and cobalt (Zn, Co): 0.3 ppm |
| | Transition and other metals ( Cd, Cr, Al, Mn, Mo, Sn, Ti, V . . . ): 1 |
| | ppm on a case by case |
| | Iron (Fe): 10 ppm |
| | Family I and II (K, Ba, μg, Be . . . ): 1000 ppm |
| | The metal coins were analyzed on a batch per batch basis by dissolution in |
| | nitric acid to assess the metal contamination within the coin that were not |
| | found in the starting nickel metal and thus originate from the process. |
| | The amount suggested above were a good, albeit not strict, guide since |
| | chemical purification following irradiation will, in turn, further remove |
| | some of these impurities. The ultimate specification on this front will |
| | therefore be an iterative process as the Cu/Ni separation chemistry is |
| | refined. However, the process shall not significantly add impurities that |
| | were not in the originating pure nickel material. |
| | It is preferable that cold Cu should be minimized in the deposited Ni since |
| | this will follow the chemistry of any ⁶¹Cu and cannot be separated post- |
| | irradiation. Any such cold Cu will directly compete with ⁶¹Cu during |
| | radiolabeling of the pharmaceutical. Methods of removing Cu from the |
| | dissolved target metal are well known. |
| Density of Target | To withstand the deposited beam power, the Ni target should preferably be |
| | of reasonably high volumetric density (e.g. approximately ≥90% or, ≥8.0 |
| | g/cm³). |
| Power Rating | The power rating for the target, including the combined deposited Ni and |
| | plate should preferably be: |
| | ≥420 W (deuterons) |
| | ≥820 W (protons) |
| Loading Mass of | The loading mass vs. the deposited mass of Ni (i.e. deposition efficiency) |
| Target | relates not to technical specifications, but rather, to cost. In the case of ^{nat}Ni |
| | deposition, loading efficiency will not have a significant impact on the cost |
| | of ⁶¹Cu. However, losses should be minimized in considering the translation |
| | to enriched ^{6x}Ni. For ⁶⁰Ni, losses should be maintained below ~10%, and |
| | for ⁶¹Ni, below ~1%. Some techniques such as magnetron sputtering are |
| | thus not possible for enriched nickel but are satisfactory for ^{nat}Ni. |
| Mass/thickness | For deuterons (i.e. ^{nat}Ni or ⁶⁰Ni), the thickness should be appropriate for |
| of Nickel | stopping the deuterons, with a maximum 10% variability in material |
| | deposition. Such thicknesses equate to: |
| | ≥100 μm (assuming 100% density) |
| | ≥70 mg or ≥89 mg/cm² (assuming 10 mm diameter) |
| | For protons (i.e. ⁶¹Ni), one may wish to selectively limit the deposited |
| | material to optimize the balance between material cost, yield, and backing |
| | material activation. With a maximum 10% variability in material |
| | deposition, four examples are noted below. |
| | ⁶¹Ni Scenario #1 (11→9 MeV) |
| | 78 μm (assuming 100% density) |
| | 55 mg or 69 mg/cm² (assuming 10 mm diameter well) |
| | ⁶¹Ni Scenario #1 (12→8 MeV) |
| | 155 μm (assuming 100% density) |
| | 108 mg or 138 mg/cm² (assuming 10 mm diameter well) |
| | ⁶¹Ni Scenario #1 (13→7 MeV) |
| | 233 μm (assuming 100% density) |
| | 163 mg or 208 mg/cm² (assuming 10 mm diameter well) |
| | ⁶¹Ni Scenario #1 (13→4 MeV) |
| | 309 μm (assuming 100% density) |
| | 216 mg or 275 mg/cm² (assuming 10 mm diameter well) |
| Isotopic | The ^{6x}Cu radioisotopes which will be coproduced during production of 61Cu |
| enrichment | (t½ = 3.339 h) include: |
| | ⁵⁷Cu (t½ = 0.196 s) ⁵⁸Cu (t½ = 3.204 s) |
| | ⁵⁹Cu (t½ = 81.5 s) ⁶⁰Cu (t½ = 23.7 m) |
| | ⁶²Cu (t½ = 9.673 m) ⁶⁴Cu (t½ = 12.701 h) |
| | From a practical handling point of view, all but ⁶⁰Cu and ⁶⁴Cu are likely to |
| | have decayed prior to use. Only ⁶⁴Cu will have any impact on the possible |
| | shelf-life of ⁶¹Cu. |
| | In addition to the production of Cu radioisotopes, other radionuclides (e.g. |
| | Co and Ni) will also be produced, the ratio of which will depend on the |
| | isotopic composition, and whether undergoing deuteron or proton |
| | irradiation. As these byproducts are chemically different from copper, such |
| | radionuclides may be removed during ⁶¹Cu purification/processing. For |
| | example, The Cu-61 was purified from metal and radiometal impurities via |
| | a GE Healthcare FASTlab 2 module through a tributyl phosphate resin |
| | cartridge and a tertiary-amine-based weak ionic exchange resin containing |
| | long-chained alcohols. |
| Any other | Niobium is preferred over silver for its better resistance to corrosion, its low |
| requirements | amount of activation on irradiation and for its high melting temperature that |
| | permits the deposit of nickel by other processes such as melting or heat |
| | sintering. However, silver possesses a higher thermal conductivity and may |
| | be suitable for certain embodiments. |
| | For target backing manufacture, the following sheet of niobium is suitable |
| | for laser cutting: |
| | http://www.Goodfellow.com |
| | NB000400 Niobium Foil, Size: 150 × 150 mm Thickness: 1.5 mm, |
| | Purity: 99.9%, Temper: Annealed, Quality: LT |
| | From one sheet up to 25 target backings can be manufactured. |

5.1.5. Purification and Characterization of [⁶¹Cu]CuCl₂ and Waste Streams

The solid target irradiated material was dissolved in a total volume of 7 mL of 6 M HCl with the addition of 30% hydrogen peroxide via a dissolution chamber.

Separation and purification was accomplished using a cassette-based FASTlab platform using a TBP (tributylphosphate-based) resin (1 mL) (particle size 50-100 μm; pre-packed, Triskem) then a weakly basic (tertiary amine; TK201) resin (2 mL) (particle size 50-100 μm; pre-packed, Triskem), each of which were pre-conditioned with H₂O (7 mL) and HCl (10M, 7 mL). The cassette reagent vials were prepared using concentrated HCl (Optima Grade, Fischer Scientific), NaCl (ACS, Fischer Scientific) and milli-Q water (Millipore system, 18 MΩ-cm resistivity). 6M HCl (2×4.2 mL), 5M NaCl in 0.05 M HCl (4.2 mL). The subsequent ⁶¹Cu was then purified with two subsequent ion exchange resins in a FASTlab synthesis unit.

1) The acid-adjusted dissolution solution (approx. 7 mL) was loaded over both columns in series and directed into a “Ni collection fraction”. The TBP resin acted as a guard column as it quantitatively retained Fe3+ ions, while the Cu²⁺ and Co²⁺ complexes were quantitatively retained on the tertiary amine (TK201) resin.

2) Both columns were washed with 6M HCl (4 mL) to maximize Ni recovery for future recycling.

3) The TK201 column was washed with 4.5M HCl (5.5 mL) to elute the majority of cobalt salts.

4) The TK201 column was washed with 5M NaCl in 0.05M HCl (4 mL) to decrease residual acid on the resin and further remove any residual cobalt salts.

5) The TK201 column was washed with of 0.05M HCl (3 mL) to quantitatively elute the [⁶¹Cu]CuCl₂.

The resulting [⁶¹Cu]CuCl₂ solution of the plated material has an average activity of 1.0-4.5 GBq. This activity was measured using a dose calibrator from Comecer and its radionuclidic purity by a gamma spectrometer at PSI in Switzerland.

Gamma spectrometry measurements were performed to identify any radionuclidic impurities, particularly long-lived radionuclides. These results indicate a 89.3% and 94% reduction in impurities for ^{nat}Ni and ⁶¹Ni on niobium backing materials with respect to silver backing materials when utilizing the methods disclosed herein. ICP-MS measurements were performed on the product of cold dissolutions by Labor Veritas in Switzerland to monitor elemental impurities present in product according to ICH-Q3D. All detected impurities were within regulated ICH-Q3D concentrations (see ICH-Q3D Guidelines, page 25).

The plating of highly enriched ⁶¹Ni was also enabled with the same plating parameters as described above, for a higher yield and industrial production using proton irradiation (typically at 80 μA to 100 μA, 13 MeV protons for 1 hour to 2 hours and up to one half-life of ⁶¹Cu).

5.1.6. Purity and Activity Evaluations of [⁶¹Cu]CuCl₂ Compositions Prepared From ^{nat}Ni (d,n) ⁶¹Cu and ⁶⁰Ni (d,n) ⁶¹Cu Using Nb-Backed Coins.

This example presents information on the activity of the produced ⁶¹Cu generated using the Nb backing, Ni electrodeposited coins of the present disclosure; alongside cobalt radioisotopes, that were produced with deuteron irradiation using the coin comprising a natural nickel target and the coin comprising enriched ⁶⁰Ni as target, i.e., ^{nat}Ni (d,n) ⁶¹Cu and ⁶⁰Ni (d,n) ⁶¹Cu, respectively. The irradiated materials were dissolved and purified as described above.

The obtained and purified ⁶¹Cu product and waste generated during purification from the products of deuteron irradiation of natural nickel/Nb coin and ⁶⁰Ni/Nb coin, respectively, was processed and analyzed by gamma-spectrometry and presented below.

TENDL-2019 is based on thick target yield calculations using isotopic abundancy of natural nickel/Nb coin and enriched ⁶⁰Ni/Nb coin, respectively.

### 5.1.7. Radiocobalt Content

Table 5 contains activities of cobalt radioisotopes in the different fractions post FASTlab purification as a mean of three measurements (n=3 irradiations) using ^{nat}Ni/Nb target coin. The activities were extrapolated to a 3 h and 50 μA beam at EoB (end of bombardment)+2 h. The activity of [⁶¹Cu]CuCl₂ in these irradiations was determined experimentally and confirmed to be ~80% of TENDL-2019 based estimates.

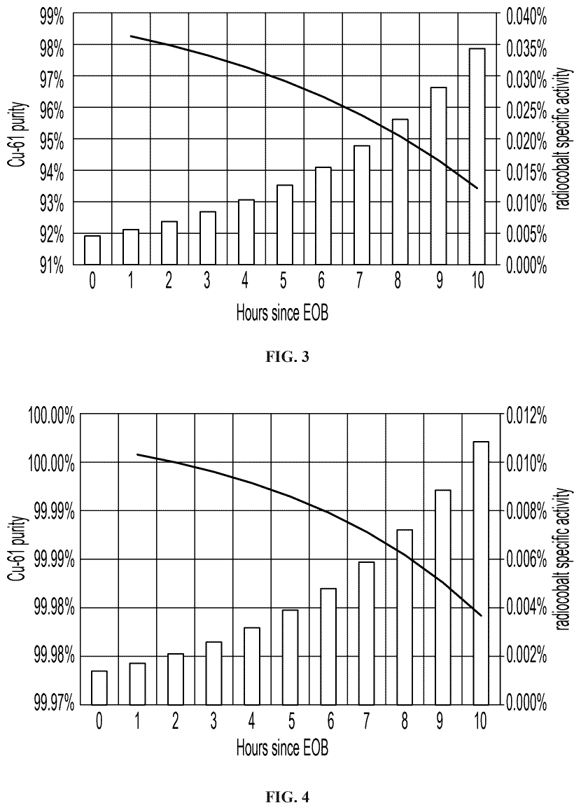

Activity of produced ⁶¹Cu for irradiation with deuteron at 8.4 MeV, 3 h at 50 μA at 80% efficiency (EoB+2 h): 3052 MBq. Also see FIG. 3 for the change in cobalt radioisotopes with time along with the corresponding change in ⁶¹Cu purity.

TABLE 5
Cobalt isotopes: natNi/Nb target coin

| | Cu fraction | Ni fraction | Co-waste | Half-life |
|---|---|---|---|---|
| Radionuclide | [Bq] | [Bq] | I + II [Bq] | [days] |
| ⁵⁶Co | 118345 | 2696 | 2458071 | 77 |
| ⁵⁷Co | 0 | 0 | 474 | 272 |
| ⁵⁸Co | 95395 | 2145 | 1940192 | 71 |
| ⁶⁰Co | 124 | 3 | 2602 | 1925 |

Table 6 contains calculated activities of cobalt radioisotopes that would be obtained by using 99% enriched ⁶⁰Ni as target metal. The activities were extrapolated to a 3 h and 50 μA beam at EoB+2 h. The activity of ⁶¹Cu was calculated accordingly.

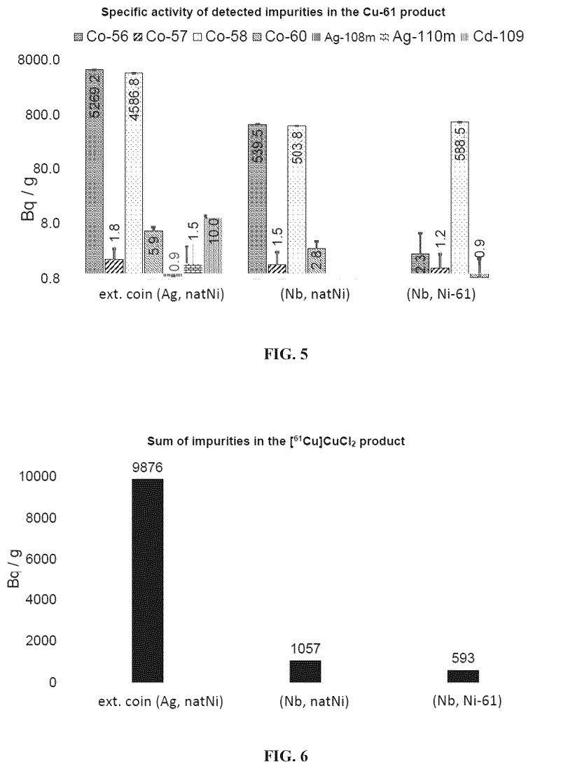

The activity of produced ⁶¹Cu with deuteron irradiation at 8.4 MeV, 3 h at 50 μA at 80% efficiency (EoB+2 h) was 11.552 MBq. Also see FIG. 4 for the change in cobalt radioisotopes with time and the corresponding change in ⁶¹Cu purity.

TABLE 6
Cobalt isotopes: enriched ⁶⁰Ni/Nb target coin.

| | | | Separated | |
|---|---|---|---|---|
| | ⁶¹Cu fraction | Separated | Co-waste | Half-life |
| Radionuclide | [Bq] | Ni [Bq] | I + II [Bq] | [days] |
| ⁵⁶Co | 365 | 8 | 7583 | 77 |
| ⁵⁷Co | 0 | 0 | 1793 | 272 |
| ⁵⁸Co | 242909 | 5463 | 4940424 | 71 |
| ⁶⁰Co | 0.5 | 0 | 11 | 1925 |

### 5.1.8. Activity and Chemical Purity

Based on measured activities (MBq) at different beam currents (μA) and timescales (5-60 minutes), the measured activity resulting from deuteron bombardment of ^{nat}Ni, ⁶⁰Ni and proton bombardment of ⁶¹Ni using the process described herein was found to be approximately >80% of the theoretical activity calculated using the TENDL-19 cross section database.

The activity of radiocobalt and other long-lived radionuclides was measured post-release (>3 weeks after bombardment). The EOB activity of the long-lived impurities was then extrapolated.

In Table 7, the extrapolated radiocobalt activity content and ⁶¹Cu purity of [⁶¹Cu]CuCl₂ solution produced by ^{nat}Ni as target metal for a 50 μA, 3 h deuteron irradiation after FASTlab purification were presented.

TABLE 7
Natural Ni/Nb Target Coin - Extrapolation of ⁶¹Cu activity
and purity in produced [⁶¹Cu]CuCl₂ solution.

| | Co species | | | | | |
|---|---|---|---|---|---|---|
| Hours | activity in | ⁶¹Cu | ⁶⁴Cu | | % Purity PET | % non-Cu |
| post | Cu fraction | activity | activity | % Purity | nuclides | radio- |
| EoB | [Bq] | [MBq] | [MBq] | ⁶¹Cu | ⁶¹Cu + ⁶⁴Cu | nuclides |
| 0 | 213864 | 4622 | 70 | | 99.995% | 0.00456% |
| 1 | 213784 | 3756 | 66 | 98.261% | 99.994% | 0.00559% |
| 2 | 213704 | 3052 | 63 | 97.979% | 99.993% | 0.00686% |
| 3 | 213624 | 2479 | 59 | 97.652% | 99.992% | 0.00841% |
| 4 | 213544 | 2015 | 56 | 97.274% | 99.990% | 0.01031% |
| 5 | 213465 | 1637 | 53 | 96.837% | 99.987% | 0.01263% |
| 6 | 213385 | 1330 | 50 | 96.332% | 99.985% | 0.01545% |
| 7 | 213305 | 1081 | 48 | 95.750% | 99.981% | 0.01890% |
| 8 | 213225 | 878 | 45 | 95.081% | 99.977% | 0.02309% |
| 9 | 213145 | 714 | 43 | 94.312% | 99.972% | 0.02817% |
| 10 | 213066 | 580 | 41 | 93.432% | 99.966% | 0.03434% |

Less than 0.03% non-Cu radioisotopes (⁵⁶Co and ⁵⁸Co) will be left in the copper fraction, assuming a product expiry time, e.g., >3 weeks post EoB. This value was lower than the limit allowed for Ga-68 cyclotron-produced as found in the Pharmacopeia (*0.1% at expiry for non-Ga radioisotopes):

The ⁶⁴Cu originating from ^{nat}Ni irradiation (content ~5% at expiry) will be the main impurity, reducing the radioisotopic purity of ⁶¹Cu product at longer irradiation times or shelf-life (illustrated as the grey curve in FIG. 3).

Table 8 and FIG. 4 show the extrapolated radiocobalt activity content and ⁶¹Cu purity of the produced [⁶¹Cu]CuCl₂ solution after FASTlab purification.

TABLE 8
⁶⁰Ni/Nb Target coin - Extrapolation of ⁶¹Cu activity
and purity in produced [⁶¹Cu]CuCl₂ solution.

| | Co species | | | | | |
|---|---|---|---|---|---|---|
| Hours | activity in | ⁶¹Cu | ⁶⁴Cu | | % Purity PET | % non-Cu |
| post | Cu fraction | activity | activity | % Purity | nuclides | radio- |
| EoB | [Bq] | [MBq] | [MBq] | ⁶¹Cu | ⁶¹Cu + ⁶⁴Cu | nuclides |
| 0 | 243275 | 17498 | 0.378 | | 99.999% | 0.00139% |
| 1 | 243176 | 14217 | 0.358 | 99.996% | 99.998% | 0.00171% |
| 2 | 242977 | 11552 | 0.339 | 99.995% | 99.998% | 0.00210% |
| 3 | 242680 | 9386 | 0.321 | 99.994% | 99.997% | 0.00259% |
| 4 | 242285 | 7627 | 0.304 | 99.993% | 99.997% | 0.00318% |
| 5 | 241792 | 6197 | 0.288 | 99.991% | 99.996% | 0.00390% |
| 6 | 241201 | 5035 | 0.272 | 99.990% | 99.995% | 0.00479% |
| 7 | 240514 | 4091 | 0.258 | 99.988% | 99.994% | 0.00588% |
| 8 | 239731 | 3324 | 0.244 | 99.985% | 99.993% | 0.00721% |
| 9 | 238853 | 2701 | 0.231 | 99.983% | 99.991% | 0.00884% |
| 10 | 237882 | 2195 | 0.219 | 99.979% | 99.989% | 0.01084% |

Less than 0.01% non-Cu radioisotopes (⁵⁶Co and ⁵⁸Co) were left in the Cu fraction, assuming a product expiry time of 8 h post EoB. This value was ten times lower than the allowed limit for ⁶⁸Ga cyclotron-produced as found in the Pharmacopeia (0.1% at expiry for non-Ga radioisotopes*).

Less than 0.02% ⁶⁴Cu was left in the copper fraction at an expiry time of 8 h post EoB, one hundred times lower than the specification required for ⁶⁸Ga (2% Ga radioisotopes were allowed for ⁶⁸Ga).

5.1.9. Purity of Produced [⁶¹Cu]CuCl₂ from Ni/Nb Target Coins: Comparison with Commercially Available Radionuclides

In Table 9, a comparison of the regulatory specifications on the purity of commercially available radionuclides are given along with the characteristics of the high purity [⁶¹Cu]CuCl₂ produced from deuteron irradiation of ^{nat}Ni/Nb and enriched ⁶⁰Ni/Nb target coin (50 μA, 3 h) and after FASTlab purification described herein.

TABLE 9
Comparison between commercially available radionuclides and [⁶¹Cu]CuCl₂
solution produced from irradiation of ^{nat}Ni/Nb coins and enriched ⁶⁰Ni/Nb coins.

| | | % Max | | | | |
|---|---|---|---|---|---|---|
| | | radioisotopes | % Max other | | | |
| | % Purity | of same | radioisotopes | | | % Max other |
| Radio- | at EoB + | element at | at EoB + 2 | Dominant | % Purity | radioisotopes |
| nuclide | 2 hours | EoB + 2 hours | hours | impurities | at expiry | at expiry |
| ¹¹¹In | 99.93% | 0.075% | | ⁶⁵Zn, ^{114m}In | 99.85% | 0.15% |
| ¹⁸F | | | | ⁵⁶Co | 99.90% | 0.10% |
| ¹⁸F | | | | ⁵⁶Co | 99.99% | 0.01% |
| ⁶⁸Ga | 98% | 2% | 0.10% | | | |
| cyclotron | | | | | | |
| ⁶⁸Ga | 99.90% | | 0.001% | ⁶⁸Ge | | |
| generator | | | | | | |
| ¹⁷⁷Lu | 99.90% | 0.05% | | | | |
| ⁶¹Cu from | 97.27% | 3.16% | 0.013% | ⁵⁶Co, ⁵⁸Co | 95.08% | 5% |
| ^{nat}Ni | (EoB + 4 h) | | | | (EoB + 8 h) | |
| ⁶¹Cu from | 99.99% | 0.009% | 0.004% | ⁵⁶Co, ⁵⁸Co | 99.98% | 0.02% |
| ⁶⁰Ni | (EoB + 4 h) | | | | (EoB + 8 h) | |

As the first notable comparison, cyclotron production of ⁶⁸Ga from proton irradiation also produces long lived radionuclides, (see, e.g., Applied Radiation and Isotopes, 65 (10), 1101-1107, IAEA-TECDOC-1863 Gallium-68 Cyclotron Production) notably ⁶⁵Zn (half-life=244 days) from the ⁶⁶Zn (p,pn) ⁶⁵Zn decay. With a roughly 0.365% of ⁶⁶Zn in an enriched ⁶⁸Zn starting target metal, about 770 Bq of ⁶⁵Zn will be produced from a 50 μA, 3 h beam with an energy of 13 MeV in a thick target (TENDL-2019 based calculations). Using natural Zn with 27.7% abundancy in ⁶⁶Zn, 58 kBq of ⁶⁵Zn will be produced in one run of 50 μA for 3 h beam.

Similar with [⁶¹Cu]CuCl₂ production, cyclotron production of [⁶⁴Cu]CuCl₂ from proton irradiation also produces long-lived cobalt radionuclides, namely, ⁵⁵Co, ⁵⁷Co, ⁵⁸Co, and ⁶⁰Co. (See, e.g., Nuclear Medicine & Biology, Vol. 24, pp. 35-43, 1997; Applied Radiation and Isotopes 68 (2010) 5-13). By operating with a degraded beam of below 13 MeV, ⁶⁰Co (from ⁶⁴Ni(p,na)⁶⁰Co) was reduced to 1 Bq per run of 50 μA, 3 h. With beam energies below 13 MeV, ⁵⁵Co, formed from the ⁵⁸Ni(p,a)⁵⁵Co reaction, will remain the main impurity (half-life=17.53 hours). The 170 Bq of the long-lived ⁵⁷Co was formed in about 170 Bq in these conditions mostly from ⁶⁰Ni(p,a)⁵⁷Co.

Note: These estimates were computed from thick target yields using TENDL-2019 cross section data and isotopic abundancy of enriched ⁶⁴Ni as follows: 0.00376% ⁵⁸Ni, 0.00298% ⁶⁰Ni, 0.0058% ⁶¹Ni, 0.135% ⁶²Ni, 99.858% ⁶⁴Ni.

### 5.1.10. Enriched ⁶¹Ni as Target Metal on Nb Backed Coins

⁶¹Cu was produced through the proton bombardment of ⁶¹Ni electroplated Nb backed coin via cyclotron equipped with a solid target system irradiating a highly pure Niobium coin plated with highly pure ⁶¹Ni (purity 99.42%). The proton beam currents used were up to 100 μA, and beam energy of 13 MeV. An aluminum beam degrader was used.

The solid target irradiated material was dissolved in a total volume of 7 mL of 6M HCl with the addition of 30% H₂O₂ in a heated dissolution chamber. The ⁶¹Cu was purified from metal and radiometal impurities via a GE Healthcare FASTlab 2 module through a tributyl phosphate resin cartridge and a tertiary-amine-based weak ionic exchange resin containing long-chained alcohols. The product was finally eluted in an ISO class 5 environment in 3 mL 0.05 M HCl through a sterile filter Millex 4 mm Durapore PVDF 0.22 μm into a sterile evacuated vial. The vial was handled with care using the appropriate shielding and can be stored at room temperature until use using appropriate shielding for transport and handling. The properties of the [⁶¹Cu]CuCl₂ solution were determined and are displayed below in Table 10.

TABLE 10
[⁶¹Cu]CuCl₂ produced from ⁶¹Ni.

| Parameter | Test Method | Specification | |
|---|---|---|---|
| Appearance | Visual inspection | Clear, colorless solution, | |
| | | free from particulate matter | |
| Volume | Weight measurement | 3 ± 0.3 | mL |
| Activity | Dose calibrator | 0.20-2 | GBq/mL |
| concentration (EoP) | | | |
| pH value | pH paper strips | 1-1.6 | |
| Radiochemical purity | Radio-TLC | ≥99% (as [⁶¹Cu]CuCl₂) | |
| Radionuclidic identity | γ-Spectrometry (in lab | γ-photons with energy peaks at: | |
| | at EoB + 90 minutes) | 283 keV ± 20 keV | |
| | | 511 ± 20 keV (eventually sum | |
| | | peak at 1022 keV ± 20 keV) | |
| | | 656 keV ± 20 keV | |
| | Half-life via dose calibrator | 200 ± 20 | min |
| Radionuclidic purity | γ-Spectrometry (sent out, | ≥99.9% | |
| | evaluated >3 weeks, values | | |
| | extrapolated to in lab at | | |
| | EoB + 90 minutes) | | |
| Bacterial endotoxin | LAL test (Endosafe) | ≤17.5 | EU/mL |
| content | | | |

| | | |
|---|---|---|
| Chemical purity | ICP-MS (sent out, evaluated >3 weeks, | Sum of impurities ≤ 15 μg/GBq |
| | values relevant to in lab at EoB + 90 | Cu ≤ 0.5 μg/GBq |
| | minutes as these do not change with | Al ≤ 2 μg/GBq |
| | time) | Co ≤ 1 μg/GBq |
| | | Fe ≤ 3 μg/GBq |
| | | Pb ≤ 1 μg/GBq |
| | | Ni ≤ 2 μg/GBq |
| | | Zn ≤ 1 μg/GBq |

*post-release (≥3 weeks)
^{#}measured periodically

As shown in Table 11 and FIG. 5, commercially available [⁶¹Cu]CuCl₂ contains radionuclidic impurities, particularly high levels of ⁵⁶Co and ⁵⁸Co, in addition to ^{110m}Ag and ¹⁰⁹Cd. Elimination of Ag and Cd isotopes from the Cu-61 product was achieved by replacing silver with niobium as backing material. There was a nine-fold reduction of 56Co isotopes for ^{nat}Ni and >2000× reduction for Ni-61 (less shielding of radioactive waste is required). 50% reduction of long-lived cobalt isotopes (earlier final disposal of the produced waste) was also observed. It was clear from the data below, that the radionuclidic purity of [⁶¹Cu]CuCl₂ produced by the methods described above to be superior to previously known methods and products. The high levels of long-lived Co, Ag, and Cd radionuclides pose a radiation burden for the patient and a radioactive waste issue for consumables that have come in contact with the [⁶¹Cu]CuCl₂ product during radiopharmaceutical manufacturing and radiolabeling.

TABLE 11
Detailed radionuclidic impurities present in commercially
available ⁶¹Cu compared to high-purity [⁶¹Cu]Cl₂
of the present disclosure, expressed in Bq/g.

| | | | Coin | | |
|---|---|---|---|---|---|
| | | | Ext. Coins | Present Coins | |
| | | | nat-Ni on Ag | nat-Ni on Nb | ⁶¹Ni on Nb |
| Elements | T_{1/2} | | Bq/g | | |
| Co-56 | 77.23 | days | 5269.2 | 539.5 | 2.3 |
| Co-57 | 271.74 | days | 1.8 | 1.5 | 1.2 |
| Co-58 | 70.86 | days | 4586.8 | 503.8 | 588.5 |
| Co-60 | 5.27 | years | 5.9 | 2.8 | 0.9 |
| Ag-108m | 439 | years | 0.9 | N/D | N/D |
| Ag-110m | 249.86 | days | 1.5 | N/D | N/D |
| Cd-109 | 462.6 | days. | 10 | N/D | N/D |

The total radionuclidic impurity profile was summed (Table 12 and FIG. 6). There was an 83% decrease in radionuclidic impurities. When present in the [⁶¹Cu]CuCl₂ product, these impurities can cause a radiation burden for the patient, waste issues, and degrade the quality of, e.g., a radiotracer or radiopharmaceutical. They can also interfere with the chelation process by competing with ⁶¹Cu, which affects the accurate radiolabeling of the tracer. An 89.3% reduction of impurities was observed upon changing the backing material from silver to the niobium backing provided herein and using the Ni plating methods described herein. An additional reduction of 46% was observed when using Ni-61 as starting material.

TABLE 12
Radionuclidic impurities in the produced [⁶¹Cu]CuCl₂.

| Coin | |
|---|---|
| Ext. Coins | Present Coins |

| | natNi on Ag. | nat-Ni on Nb | ⁶¹Ni on Nb |
|---|---|---|---|
| Bq/g | 9876 ± 1.5 | 1057 ± 1.8 | 593 ± 0.2 |

Consequent to the purity of the ⁶¹Cu at EoB and End of Production (EoP, EoB+2 hours), long-lived radionuclidic impurities decay slower and, thus, increase in concentration in relation to ⁶¹Cu at longer timescales. Thus, the impurity profile may vary greatly based on the isotopic enrichment of the raw material, purity, method, and process of producing a coin, which influences the type and amount of radionuclidic impurities in the finished [⁶¹Cu]CuCl₂ product.

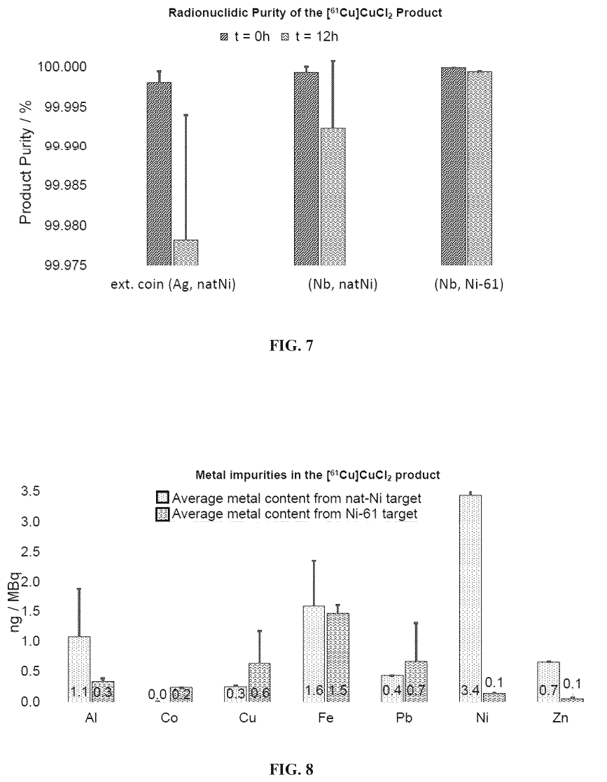

FIG. 7. contrasts the radionuclidic purity of [⁶¹Cu]CuCl₂ solution produced with commercially available natNi target metal on a Ag backing compared to the radionuclidic purity of [⁶¹Cu]CuCl₂ solution produced by irradiation of Ni target metal electroplated according to the present disclosure on high purity Nb backing when assessed by gamma spectrometry in Bq/g (summed radionuclidic impurities) at t=0 h and at t=12 h. The presented data highlight the superior quality of the [⁶¹Cu]CuCl₂ solution when produced by irradiation of Ni target coatings electroplated according to the present disclosure on high purity Nb backing, where the purity after 12 hours is still well above the purity limits set by pharmacopeia for similar radionuclides for medical use. End of Production” or “EoP” refers to the end of the preparation of [⁶¹Cu]CuCl₂.

TABLE 13
Radionuclidic purity of commercially available ⁶¹Cu
compared to high-purity [⁶¹Cu]CuCl₂ of the present
disclosure as measured at EoP and EoP + 12 hours.

| Coin | |
|---|---|
| Ext. Coins | Present Coins |

| | nat-Ni on Ag | nat-Ni on Nb | ⁶¹Ni on Nb |
|---|---|---|---|
| Purity % t = 0 h | 99.998 | 99.999 | 99.9999 |
| Purity % t = 12 h | 99.978 | 99.993 | 99.999 |

### 5.1.11. Conclusion

The experimental activities of ⁶¹Cu produced after deuteron irradiation were about 80% of the theoretical yield as calculated from TENDL-2019 cross section data.

The main long-lived nuclides in the radioactive waste fraction from cyclotron production of ⁶¹Cu were radiocobalt species of ⁵⁶Co, ⁵⁷Co, ⁵⁸Co, and ⁶⁰Co. It was calculated that, after four years, ⁵⁶Co, ⁵⁷Co, and ⁵⁸Co will have decayed below regulatory clearance limits, LL*, leaving only ⁶⁰Co. *Clearance limits (LL) means the value corresponding to the activity concentration level of a material below which handling of this material is no longer subject to mandatory licensing or supervision.

The yield and purity of [⁶¹Cu]CuCl₂ prepared with niobium coins was improved by plating the niobium coins with 99% enriched ⁶⁰Ni or ⁶¹Ni. The purity of [⁶¹Cu]CuCl₂ product was higher as ⁶⁴Cu was be formed as a radioisotopic impurity. Additionally, the ⁵⁶Co and ⁶⁰Co contents were reduced by a factor of 100. ⁵⁷Co amounts increased (but were low activity) and ⁵⁸Co amounts doubled (but decay below LL before ⁵⁶Co/⁵⁸Co).

5.1.12. Batch Control of [⁶¹Cu]CuCl₂

Three representative batches of [⁶¹Cu]CuCl₂ solution were manufactured as described above (by irradiating a solid target consisting of highly pure ⁶¹Ni (purity>99.42%) plated on a niobium coin backing with a proton beam) and tested. The results of these analyses are presented in Table 14. “End of Production” or “EoP” refers to the end of the preparation of [⁶¹Cu]CuCl₂.

TABLE 14
Analyses of three batches of [⁶¹Cu]CuCl₂.

| | Specification | | Results | | | | | |
|---|---|---|---|---|---|---|---|---|
| Batch | — | | 1 | | 2 | | 3 | |
| Volume | 3 ± 0.3 | mL | 3.02 | mL | 3.00 | mL | 3.19 | mL |
| Activity | 0.50-2.00 | GBq/mL | 1.30 | GBq/mL | 1.29 | GBq/mL | 1.47 | GBq/mL |
| concentration | | | | | | | | |
| (EoP) | | | | | | | | |
| Apparent molar | 35-580 | MBq/nmol | 144.44 | MBq/nmol | 144.44 | MBq/nmol | 163.33 | MBq/nmol |
| activity | | | | | | | | |
| pH value | 1-1.6 | | 1.3 | | 1.2 | | 1.3 | |
| Radiochemical | ≥99% (as | | 100% | | 100% | | 100% | |
| purity | [⁶¹Cu]CuCl₂) | | | | | | | |
| Radionuclidic | γ-photons with | | 290 | keV | 286 | keV | 286 | keV |
| identity | energy peaks at: | | 506 | keV | 510 | keV | 510 | keV |
| | 283 keV ± 20 keV | | 654 | keV | 652 | keV | 656 | keV |
| | 511 ± 20 keV | | | | | | | |
| | (eventually sum | | | | | | | |
| | peak at 1022 | | | | | | | |
| | keV ± 20 keV) | | | | | | | |
| | 656 keV ± 20 keV | | | | | | | |
| Half-life | 200 ± 20 | min | 195.6 | min | 196.2 | min | 195.6 | min |
| Radionuclidic | ≥99.9% | | 99.99% | | 99.99% | | 99.99% | |
| purity* | | | | | | | | |
| Bacterial | ≤17.5 | EU/mL | ≤3.75 | EU/mL | ≤3.75 | EU/mL | ≤3.75 | EU/mL |
| endotoxin | | | | | | | | |
| content | | | | | | | | |

| | | | | |
|---|---|---|---|---|
| Chemical | Sum of impurities | Sum of | Sum of | Sum of |
| purity* | ≤20 μg/GBq | impurities | impurities | impurities |
| | | 2.40 μg/GBq | 2.11 μg/GBq | 2.72 μg/GBq |

*post-release.

### 5.1.13. Radionuclidic Purity

Radionuclidic purity is important in radiopharmacy since any radionuclidic impurities increase the radiation dose received by the patient and may also degrade the quality of any imaging procedure performed. For example, if significant levels of other radionuclides are present then biological distribution may be altered. Radionuclide samples contain some contaminants arising the production process or the decay of the primary radioisotope. Radionuclide impurities can occur as a result of the manufacturing process, for example, for nuclides produced by cyclotron there can be contaminants due to impurities in the target or by the energy of the reaction. In order to control the effects of these contaminants on the radiation dose received by the patient, limits are set on the maximum levels of contamination allowed. These limits are defined by governmental agencies, e.g., in pharmacopoeia monographs, and vary depending upon the radionuclide concerned and the physical decay characteristics of the likely contaminants. Measurement of radionuclidic purity may be performed high resolution using gamma-ray spectroscopy on samples well after bombardment. The activity of the long-lived isotopes is then extrapolated back to EoB, EoP, EOS, or even at expiration. High activity emitted from long lived radionuclidic impurities greatly increases the cost and complexity of managing the disposal of all consumables that come into contact with the nuclide composition.

Through the deuteron irradiation of natural nickel and ⁶⁰Ni, and proton irradiation of ⁶¹Ni, long-lived isotopes of cobalt are produced: ⁵⁶Co, ⁵⁷Co, ⁵⁸Co and ⁶⁰Co. Other long-lived radionuclides such as ^{110m}Ag, ^{108m}Ag and ¹⁰⁹Cd are produced through the irradiation of commonly used silver backing material, which are dissolved along with starting material during the purification process. Due to their long half-lives, the proportion of these radionuclides increases with time compared to the ⁶¹Cu, decreasing the radionuclidic purity of the product, especially at later time points when using ^{nat}Ni as a starting material. Though most cobalt isotopes can be separated in the purification process, the ^{110m}Ag, ^{108m}Ag and ¹⁰⁹Cd end up in the ⁶¹Cu fraction and nickel solution that is further used in recycling of irradiated target coating. The long-lived radionuclides become problematic when considering the radiation burden to the patient and the accumulation of radioactive waste. Third-party coin manufacturers did not publish the contamination from the non-niobium coin backings (e.g., silver). As provided by the present disclosure, the method of making and using coins comprising niobium represents an advantage, e.g., in view of the radionuclidic and chemical purity of samples produced following subatomic particle bombardment, isolation, and purification. A detailed comparison of the known ⁶¹Cu products (prepared via Ag backings and prior art methods of plating the target) to ⁶¹Cu as provided by the present disclosure is provided below.

With these factors in mind, a niobium backing material was chosen due to its inert nature to acids at room temperature and at elevated temperatures. This characteristic allows the niobium backing material to resist the acid medium used during the dissolution and purification process. By doing so, higher radionuclidic and chemical purity can be achieved in the radiometal aqueous solution, eventually resulting in higher purity for the radiopharmaceutical prepared from the desired ⁶¹Cu isotope. Although plating methods of niobium exist, the element has not yet been used for radionuclide production due to the poor adhesion of the plated Ni material (as discussed above). The Ni (or ⁶⁸Zn for the production of ⁶⁸Ga) requires sufficient adhesion for the coin to survive thermal loads (1200 W) during irradiation and pneumatic shuttle acceleration at 5 bar to 7 bar of pressure and abrupt stop at the head. On the other hand, however, the plated Ni (or Zn) must dissolve sufficiently during the dissolution and purification process. Attempts were made to plasma-coat niobium backings for plating nickel (Ni). However, this process resulted in losses and incomplete dissolution of Ni from the niobium backing. The thermal processes involved in plasma coating altered the grain structure of the niobium backing material, leading to a strong bond between the plated nickel and niobium. This strong bond made it difficult for the nickel to fully dissolve, causing losses. The plasma coating process itself resulted in very high losses in target coating, rendering the process not viable for use, especially with very expensive highly enriched target metals. The main reference to this summary is the IAEA documentation regarding cyclotron radionuclide production, IAEA RADIOISOTOPES AND RADIOPHARMACEUTICALS, REPORTS, No. 1. (INTERNATIONAL ATOMIC ENERGY AGENCY VIENNA, 2016) Additionally, a monetary evaluation regarding the procurement costs of niobium utilized as a backing material displays a 40% lower cost in comparison to commonly used backing materials such as gold, silver, and platinum where costs range from €80 to €120 per backing material (single coin).

Parallel to this, elements pertaining to the radiochemical purity of the labelling process are controlled by manufacturing the plating solution under controlled conditions described herein. By procuring the plating solution from a raw base material of, e.g., nickel, the possibility of contamination is now independent from outside sources and suppliers. Such material and equipment used in these cases are inert glass beakers and falcon tubes (ensured to not contain any undesirable substances), TraceSelect pure water, pure reagents (trace-metal grade), inert coin adapter and electrolytic cell (on the electroplating unit), etc. Through this, the contaminants of trace metals can be minimized reduced or avoided all together. This difference between 99.9% purity and 99.99% purity plays a role in the resulting chemical purity of a radionuclide and therefore in the radiochemical purity of, e.g., a radiotracer or radiopharmaceutical prepared from the radionuclide, where the presence of cold Cu, Zn, Fe, Sn, Ti, or Al or any salt thereof are an issue as they will compete for binding to the chelator in the tracer along with the desired radionuclide (⁶¹Cu).

Robustness of plating was tested through a drop and scratch test. This assessment ensures that the electrodeposited substrate on the backing will survive mechanical impacts of the shuttling system and established an increased probability of survivability under the cyclotron beam.

In certain embodiments, coins were irradiated with 8.4 MeV deuterons for an average duration of 120 mins at a range of 40 μA to 45 μA or with 13.2 MeV deuterons at 40 μA to 45 μA using an ARTMS or GE shuttling system on a GE PET Trace cyclotron.

In certain embodiments, the coins were irradiated with 8.4 MeV deuterons for an average duration of 120 mins at a range of 40 μA to 45 μA or with 10 μA to 100 μA 13 MeV protons using an ARTMS or GE shuttling system on a GE PET Trace cyclotron.

Dissolution of Ni from the niobium backing was accomplished via the utilization of a dissolution system in 10 M HCl. The subsequent ⁶¹Cu was then purified with two subsequent ion exchange resins in a FASTlab synthesis unit. The processing time for these purifications can reach up to 60 minutes.

The resulting [⁶¹Cu]CuCl₂ solution of the plated material has an average activity of 1.7-4.5 GBq. This activity was measured using a dose calibrator and its radionuclidic purity by a calibrated gamma spectrometer e.g., at PSI in Switzerland.

Gamma spectrometry measurements were performed to identify any radionuclidic impurities, particularly long-lived radionuclides. These results indicate an 89.3% and 94% reduction in impurities for ^{nat}Ni and ⁶¹Ni on niobium backing materials with respect to silver backing materials when utilizing the methods disclosed herein. ICP-MS measurements are performed on the product of cold dissolutions by Labor Veritas in Switzerland to monitor elemental impurities present in the product according to ICH-Q3D. All detected impurities are within regulated ICH-Q3D concentrations (see ICH-Q3D Guidelines, pg 25).

The plating of highly enriched ⁶¹Ni was also enabled with the same plating parameters as described above, for a higher yield and industrial production using proton irradiation (typically at 10 μA to 100 μA, 13 MeV protons for 20 minutes to 2 hours and up to one half-life of ⁶¹Cu).

Following automated transportation of the irradiated coin from the cyclotron to the hot cell docking station, the capsule was transferred to a QIS dissolution unit with tongs. The transmuted target metal was dissolved from the niobium backing material using 1:1 7M HCl: 30% H₂O₂ (ultratrace analysis, Merck) (4 mL). The acid-peroxide mixture is circulated, immersing the coin and target metal surface to dissolve all irradiated elements at 2 mL/min for about 23 minutes at about 60° C. When the target metal was fully dissolved, acidic solution containing the dissolved metal was withdrawn and the QIS system was flushed with 10M HCl (3 mL). The combined acidic solutions were then fed forward to the FASTlab purification unit.

For this reason, gamma spectrometry analyses were carried out on decayed samples (at least 10 half-lives of ⁶¹Cu, corresponding at the earliest to 1.4 d after EoP). The analysis was performed using a high-purity germanium (HPGe) detector GEM30-70 from Ortec.

### 5.1.14. Activity Concentration

The activity of the [⁶¹Cu]CuCl₂ solution was quantified using a dose calibrator following Ph. Eur. 2.2.66 guidelines. The test was carried out at the end of the radionuclide production (EoP) using a certified dose calibrator, selecting the ⁶¹Cu measuring channel. The weight of the [⁶¹Cu]CuCl₂ solution was measured using an analytical scale. Assuming a density of the aqueous solution of 1.0 g/mL, the weight is converted into a volume. The activity concentration, expressed in GBq/mL, is then calculated by dividing the activity by the volume.

### 5.1.15. Apparent Molar Activity

The suitability of the [⁶¹Cu]CuCl₂ solution for radiolabeling was ensured by determining the apparent molar activity. This test assessed the impact of competing trace metals on ⁶¹Cu chelation by quantifying the minimum amount of a given chelator required for efficient radiolabeling. The test can be performed with various chelators that can complex copper (e.g., NOTA, DOTA, NODAGA). Among them, NODAGA has been assessed for ⁶¹Cu molar activity. The test measures the percentage of complexation by radio-TLC after reacting a fixed amount of [⁶¹Cu]CuCl₂ solution with different amounts of chelator (titration). The results are then plotted (x axis (logarithmic): nmol of chelator; y axis: % of complexation), and a sigmoidal curve is obtained. The EC₅₀ point is extrapolated, corresponding to the amount of chelator needed to achieve the 50% of complexation. The apparent molar activity is calculated by dividing the ⁶¹Cu activity used extrapolated at the end of production (EoP) by 2 times the EC₅₀ value.

This test was not performed as a routine analysis, but when changes occur in 1) the grade of a chemical employed for the manufacturing of the [⁶¹Cu]CuCl₂ solution, 2) the quality of a consumable employed for the manufacturing of the [⁶¹Cu]CuCl₂ solution, 3) the target coin manufacturing process, and 4) if poor radiolabeling yields were observed. The AMA test is specific to a chelator and labelling conditions (e.g., buffer concentration and pH) and has thus to be repeated when these parameters are modified.

5.1.16. pH

The pH value was determined by colorimetric evaluation using pH paper strips with a narrow range (pH interval 0-2.5) exposed to a 10 μL of the [⁶¹Cu]CuCl₂ solution. The color of the paper strips was compared to the reference color scale displayed on the strips container.

### 5.1.17. Radiochemical Purity (Radio-TLC)

The radiochemical purity of a [⁶¹Cu]CuCl₂ solution prepared as described herein was determined by radio-TLC following Ph. Eur. 2.2.66 guidelines. The test determined the percentage of ⁶¹Cu present in the desired ionic form, namely as free Cu²⁺. The retention factor (R_{f}) was determined as the distance from the origin to the peak divided by the distance from the origin to the solvent front. Ionic [⁶¹Cu]Cu²⁺ migrated to the solvent front (R_{f}=0.8-1.0), while colloidal [⁶¹Cu]Cu(OH)₂ remained at the point of application (R_{f}=0.0-0.2). The specification required that ≥99% of the whole radioactivity detected was present in the ionic form [⁶¹Cu]Cu²⁺.

For the test, 2 μL of the radioactive solution was applied at 1 cm away from the lower end of an iTLC paper plate (8×1 cm), consisting of glass microfiber chromatography paper impregnated with silica gel. After drying, the iTLC plate was placed in a glass chamber where it was run in a 0.1 M citrate buffer solution (pH 5). When the solvent front had reached a distance of approximately 1 cm from the top of the iTLC plate, the plate was removed from the chamber and scanned using a PET miniGita Star from Elysia-Raytest, equipped with a β-sensitive detector and controlled by the software Gina Star from Elysia-Raytest.

### 5.1.18. Gamma Spectrometry (Radionuclidic Identity)

The radionuclidic identity of a [⁶¹Cu]CuCl₂ solution prepared as described herein was confirmed by gamma spectrometry following Ph. Eur. 2.2.66 guidelines. The presence of the main Y-photons with energy peaks characteristic of ⁶¹Cu (listed in Table 15) was assessed. The table also lists the energy peak of the γ-photons belonging to ⁵⁸Co, which represents the main impurity detected in the test. The test was carried out at EoP using a Mucha Star multichannel analyzer from Elysia-Raytest, equipped with a NaI detector, and controlled by the Gina Star from Elysia-Raytest software.

TABLE 15
Energy peaks characteristic of ⁶¹Cu and of its main impurity ⁵⁸Co.

| Peak | Energy (keV) |
|---|---|
| Cu-61 | 67 |
| Cu-61 | 283 |
| Beta peak | 511 |
| Cu-61 | 656 |
| Co-58 | 811 |
| Sum peak | 1022 |
| Cu-61 | 1185 |

### 5.1.19. Half-Life (Radionuclidic Identity)

The radionuclidic identity of a [⁶¹Cu]CuCl₂ solution prepared as described herein was further confirmed by half-life determination following Ph. Eur. 2.2.66 guidelines. The test was carried out at the end of the radionuclide production (EoP) using a certified dose calibrator from Comecer (model VDC-505), selecting the ⁶¹Cu measuring channel. The specification required the measured half-life value to be within a predefined range of the accepted half-life value (20%).

### 5.1.20. LAL Test (Bacterial Endotoxin Content)

The bacterial endotoxins were determined in a [⁶¹Cu]CuCl₂ solution prepared as described herein by limulus amoebocyte lysate (LAL) test following Ph. Eur. 2.6.14 guidelines. This system applies LAL kinetic chromogenic methodology that measures color intensity directly proportional to the endotoxin concentration in the sample. Each cartridge contained predefined amounts of LAL reagent, chromogenic substrate, and control standard endotoxin (CSE). The LAL reagent was mixed automatically by the device with the sample or the positive product control. The mixtures were incubated and then combined with the chromogenic substrate. For quantification, the optical density of the substrate was measured and analyzed against an internally archived standard curve. The analysis was performed in duplicate for the sample as well as for the positive product control. The system contained an internal printer to generate a report.

### 5.1.21. Bioburden

The bioburden of the aqueous [⁶¹Cu]CuCl₂ manufacturing process described herein was evaluated following Ph. Eur. 2.6.12 guidelines. This test enables the detection and quantification of the viable microorganisms present in the system prior to terminal sterilization and represents a good reference point for evaluating the degree of safety of the process. The manufacturing process was performed in completeness but used non-irradiated target coins for radiation protection. The collected non-radioactive solution was analyzed by the Membrane-Filtration Method. Half of the sample was passed through a membrane filter with a pore size of 0.45 μm. The filter was placed onto Soybean-Casein Digest Agar and incubated to determine the total aerobic microbial count (TAMC). The other half of the sample was passed through a membrane filter with a pore size of 0.45 μm. The filter was then placed onto Sabouraud Dextrose Agar and incubated to determine the total yeast and mold count (TYMC). The bioburden was expressed in colony-forming units (CFU).

### 5.1.22. Control of Starting Material

Reagents and starting materials used to manufacture the radionuclide ⁶¹Cu are provided in Table 16. The reagents employed in production were of TraceSelect grade to minimize the trace metal impurities. These impurities could impact radiolabeling, which may result in a poor complexation of ⁶¹Cu with the chelator.

TABLE 16
Materials used to purify and formulate [⁶¹Cu]CuCl₂.

| Material | Quality |
|---|---|
| ⁶¹Ni | 99.42% enrichment |
| Niobium coin backing | 99.9% purity |
| Hydrochloric acid | 30%, Ultrapure |
| Hydrogen peroxide | 30%, Suprapure |
| Sodium chloride | 99.99%, Suprapure |
| Water | Ultrapure |
| FASTlab cassette | Cassette's plastic components either |
| | sterile or tested for low bioburden |
| Solid Target Valve Box | Cassette's plastic components either |
| (STVB) cassette | sterile or tested for low bioburden |
| TBP and TK201 | Ion-exchange cartridge for purification |
| cartridges | |
| FASTlab water bag | Sterile WFI USP/Ph. Eur. |
| Vials | 20 mL sterile evacuated |
| Syringe needles | Sterile |
| Sterile filter Millex GV | 0.22 μm pore size, sterile |

At the end of the electroplating process, the target coin was examined using an optical microscope (20×, 50× and 250× magnification). All the steps of the coin manufacturing were performed using TraceSelect grade chemicals and metal-free consumables. The target coins were stored in a cool, dark environment in metal-free sealed containers.

### 5.1.23. Impurities

The impurities present in the irradiated starting material can undergo a nuclear reaction, leading to the formation of undesired radioactive species, as summarized in Table 17. Due to the high isotopic purity of the ⁶¹Ni (99.42% enrichment), these contaminations were limited.

TABLE 17
Composition of the ⁶¹Ni target coin
plating and main nuclear reactions.

| | |
|---|---|
| ⁶¹Ni composition | ⁵⁸Ni (0.004%), ⁶⁰Ni (0.3%), ⁶¹Ni (99.42%), |
| | ⁶²Ni (0.3%), ⁶⁴Ni (0.004%) |
| Desired nuclear reaction | ⁶¹Ni(p,n)⁶¹Cu |
| Contamination reactions | ⁶⁰Ni(p,α)⁵⁷Co (half-life: 271.74 d) |
| (selection of the main | ⁶¹Ni(p,α)⁵⁸Co (half-life: 70.86 d) |
| contributions) | ⁶¹Ni(p,n)⁶⁰Co (half-life: 1925 d) |

The presence of “cold” trace metal ions in the target coin material and reagents used during the dissolution and purification stages are to be limited. These trace metal ions can impact the subsequent radiolabeling processes by interfering with the complexation of ⁶¹Cu with the chelator. TraceSelect grade reagents and metal-free consumables are used with the aim of minimizing trace metal contamination.

The concentration of the trace metal ions in the [⁶¹Cu]CuCl₂ solution is determined by Inductively Coupled Plasma Mass Spectrometry (ICP-MS) (Table 18). ICP-AES can be used as alternative technique.

### 5.2. Example 2: [61Cu]Cu-NODAGA-PSMA I&T Production

General Analytical reversed-phase high performance liquid chromatography (RP-HPLC) was performed on a Nucleosil 100 C18 (5 μm, 125×4.0 mm) column (CS GmbH, Langerwehe, Germany) using a Sykam gradient HPLC System (Sykam GmbH, Eresing, Germany). The peptides were eluted applying different gradients of 0.1% (v/v) trifluoroacetic acid (TFA) in H2O (solvent A) and 0.1% TFA (v/v) in acetonitrile (solvent B) at a constant flow of 1 mL/min (specific gradients are cited in the text). UV detection was performed at 220 nm using a 206 PHD UV-Vis detector (Linear™ Instruments Corporation, Reno, USA). Both retention times tR as well as the capacity factors K′ are cited. Preparative RP-HPLC is performed on the same HPLC system using a Multospher 100 RP 18-5 (250×20 mm) column (CS GmbH, Langerwehe, Germany) at a constant flow of 9 mL/min. Radio-HPLC of the radioiodinated reference ligand is carried out using a Nucleosil 100 C18 (5 μm, 125×4.0 mm) column.

### Synthesis of carboxyl-protected Lys-urea-Glu-core (KuE)

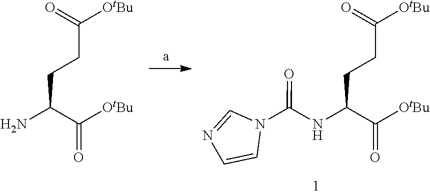 [SMILES: CC(C)(C)OC(=O)CC[C@H](N)C(=O)OC(C)(C)C ; CC(C)(C)OC(=O)CC[C@H](NC(=O)n1ccnc1)C(=O)OC(C)(C)C]

Step a. (S)-di-tert-butyl 2-(1H-imidazole-1-carboxamido) pentanedioate (1) was synthesized from the di-tert-butyl ester of glutamic acid. It was reacted with carbonyldiimidazole (CDI) under anhydrous conditions in the presence of triethylamine (TEA) to form the intermediate acylimidazole derivatives. HPLC (10% to 90% B in 15 min): tR=12.2 min; K′=5.78. Calculated monoisotopic mass for 1 (C₁₇H₂₇N₃O₅): 353.4; found: m/z=376.0 [M+Na]+.

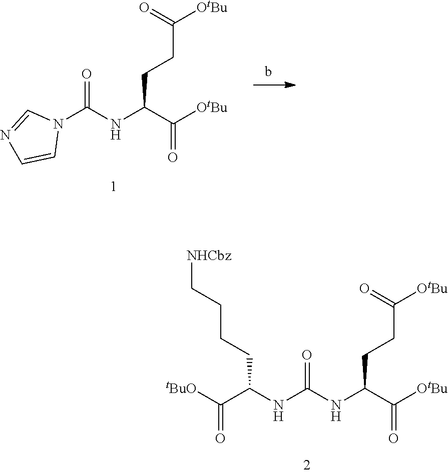 [SMILES: CC(C)(C)OC(=O)CC[C@H](NC(=O)n1ccnc1)C(=O)OC(C)(C)C ; CC(C)(C)OC(=O)CC[C@H](NC(=O)N[C@@H](CCCCNC(=O)OCc1ccccc1)C(=O)OC(C)(C)C)C(=O)OC(C)(C)C]

Step b. Cbz-(OtBu) KuE (OtBu) 2 (2): A solution of 3.40 g (9.64 mmol, 1.0 eq) 1 in 45 mL 1,2-dichloroethane (DCE) was cooled to 0° C., and 2.69 mL (19.28 mmol, 2.0 eq) of triethylamine (TEA), and 3.59 g (9.64 mmol, 1.0 eq) of Cbz-Lys-OtBu HCl were added under vigorous stirring. The reaction mixture was heated to 40° C. overnight. The solvent was removed in vacuo, and the crude product was purified via silica gel flash-chromatography using an eluent mixture of ethyl acetate/hexane/TEA (500/500/0.8 (v/v/v)). Upon solvent evaporation, 4.80 g of 2 was obtained as a colorless, sticky oil (yield: 80% based on L-di-tert-butyl glutamate HCl). HPLC (40% to 100% B in 15 min): tR=14.3 min; K′=8.53. Calculated monoisotopic mass for 2 (C₃₂H₅₁N₃O₉): 621.8; found: m/z=622.2 [M+H]+, 644.3 [M+Na]+.

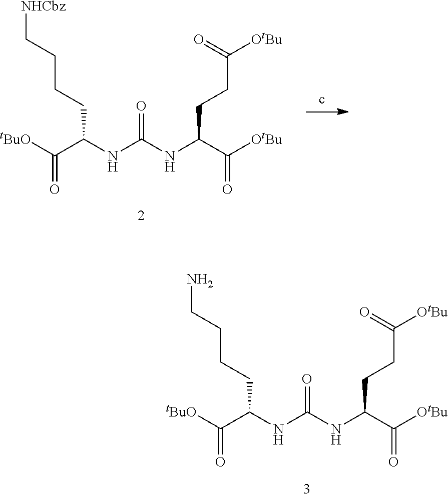 [SMILES: CC(C)(C)OC(=O)CC[C@H](NC(=O)N[C@@H](CCCCNC(=O)OCc1ccccc1)C(=O)OC(C)(C)C)C(=O)OC(C)(C)C ; CC(C)(C)OC(=O)CC[C@H](NC(=O)N[C@@H](CCCCN)C(=O)OC(C)(C)C)C(=O)OC(C)(C)C]

Step c. (OtBu)KuE(OtBu)₂ (3): For Cbz deprotection, 6.037 g (9.71 mmol, 1.0 eq) of 2 was dissolved in 150 mL of ethanol (EtOH), and 0.6 g (1.0 mmol, 0.1 eq) of Palladium on activated charcoal (10%) was added. After purging the flask with H₂, the solution was stirred overnight under light H₂-pressure (balloon). The crude product was filtered through Celite, the solvent was evaporated in vacuo, and the desired product was obtained as a waxy solid (4.33 g, 91.5% yield). HPLC (10% to 90% B in 15 min): tR=12.6 min; K′=6.41. Calculated monoisotopic mass for 3 (C₂₄H₄₅N₃O₇): 487.6; found: m/z=488.3 [M+H]⁺, 510.3 [M+Na]+.

### Synthesis of Protected Sub-KuE Conjugate

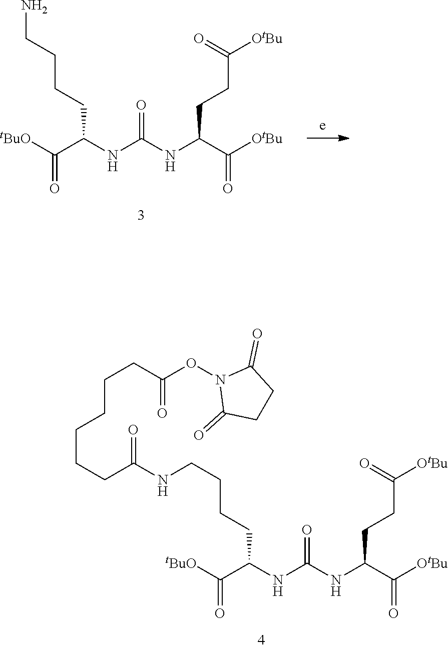 [SMILES: CC(C)(C)OC(=O)CC[C@H](NC(=O)N[C@@H](CCCCN)C(=O)OC(C)(C)C)C(=O)OC(C)(C)C ; CC(C)(C)OC(=O)CC[C@H](NC(=O)N[C@@H](CCCCNC(=O)CCCCCCC(=O)ON1C(=O)CCC1=O)C(=O)OC(C)(C)C)C(=O)OC(C)(C)C]

NHS-Sub-(OtBu) KuE (OtBu) 2 (4): 3 (40 μg, 0.08 mmol, 1 eq) was dissolved in 500 μL N,N-dimethylformamide (DMF), and 57 μL (0.41 mmol, 5 eq) of TEA was added. This solution was added dropwise (within 30 min) to a solution of 33.2 μg (0.09 mmol, 1.1 eq) of disuccinimidyl suberate (DSS). After stirring for an additional 2 h at room temperature (RT), the reaction mixture was concentrated in vacuo, diluted with ethyl acetate and extracted with water (twice). The organic phase was dried over Na₂SO₄, filtered and evaporated to dryness. Due to sufficient purity of the crude 4, it was used for the following reaction step without further purification. HPLC (10% to 90% B in 15 min): tR=16.9 min; K′=8.39. Calculated monoisotopic mass for 4 (C₃₆H₆₀N₄O₁₂): 740.4; found: m/z=741.2 [M+H]⁺, 763.4 [M+Na]+.

### Synthesis of Peptidic Linker

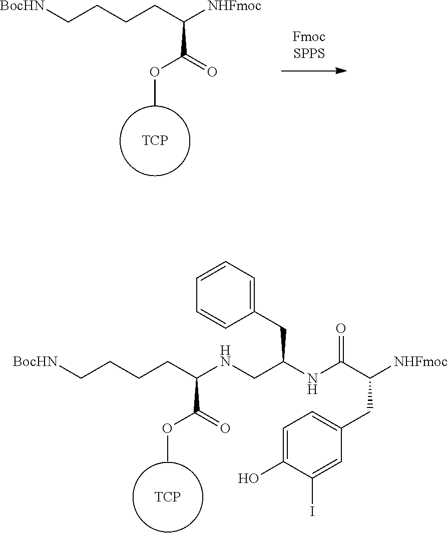

Fmoc-3-iodo-D-Tyr-D-Phe-D-Lys (Boc) (Fmoc-(I-y) fk): Fmoc-Lys (Boc)-OH (1.5 eq) was dissolved in dry dichloromethane (DCM), and N,N-diisopropylethylamine (DIPEA) (1.25 eq) was added. Dry TCP resin was suspended and stirred at RT for 5 min. Another 2.5 eq of DIPEA was added, and stirring was continued for 90 min. Then, 1 mL methanol (MeOH) per gram resin was added to cap unreacted tritylchloride groups. After 15 min, the resin was filtered off, washed twice with DCM, DMF and MeOH, respectively, and dried in vacuo. The final load of resin-bound Fmoc-Lys (Boc)-OH was calculated from the weight difference.

Assembly of the peptide sequence H₂N-3-iodo-D-Tyr-D-Phe-on resin-bound Lys (Boc) was performed according to a standard Fmoc-protocol using 1.5 eq of 1-hydroxybenzotriazole (HOBt) and O-(1H-benzotriazol-1-yl)-N,N,N′,N′-tetramethyluronium-tetrafluoroborate (TBTU) as coupling reagents and 4.5 eq DIPEA. After coupling of the last amino acid, the resin was washed, dried and stored in a desiccator until further functionalization.

### Coupling of Chelating Moiety

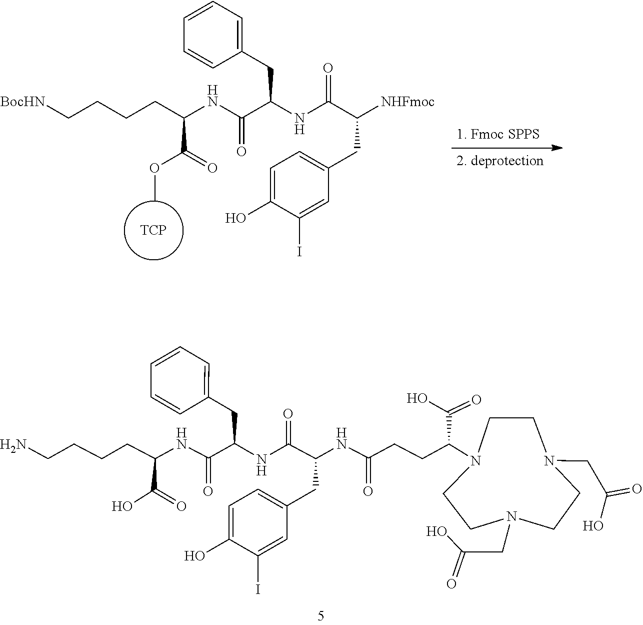

Fmoc-3-iodo-D-Tyr-D-Phe-D-Lys (Boc)-TCP resin was allowed to pre-swell in N-methyl-pyrrolidone (NMP) for 30 min. After cleavage of the N-terminal Fmoc-protecting group using 20% piperidine in DMF (v/v), the resin was washed eight times with NMP.

NODAGA-iodo-D-Tyr-D-Phe-D-Lys (NODAGA-(I-y)fk, 5): For 38 μmol of resin-bound peptide, 31 μg of NODAGA-tris-tBu-ester (57 μmol, 1.5 eq), 108 μg of O-(7-azabenzotriazol-1 -yl)-N,N,N′,N′-tetramethyluronium hexafluorophosphate (HATU; 0.28 μmol, 5 eq) and 87 μL of DIPEA (570 μmol, 15 eq) in NMP were added to the resin. After 72 h of shaking, the resin was washed with NMP and DCM. HPLC (10% to 90% B in 15 min): (tR=8.2 min; K′=4.13. Calculated monoisotopic mass for 5 (C₃₉H₅₄IN₇O₁₂): 939.29.

Cleavage from the resin (2×30 min) and concomitant tBu-deprotection was performed using a mixture (v/v/v) of 95% TFA, 2.5% triisobutylsilane (TIBS) and 2.5% water. The combined product solutions were then concentrated, the crude peptide was precipitated using diethyl ether and dried in vacuo. Due to sufficient purity of the crude products, they were used for the following reaction step without further purification.

### Condensation of the Chelator-Conjugated Peptides and the PSMA Binding Motif

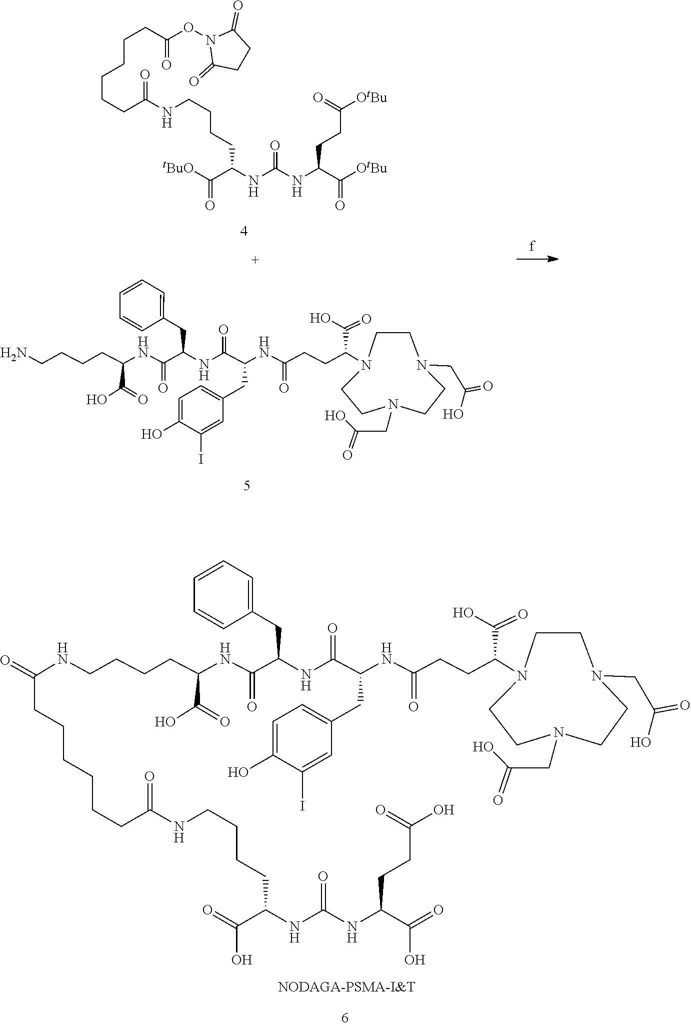 [SMILES: CC(C)(C)OC(=O)CC[C@H](NC(=O)N[C@@H](CCCCNC(=O)CCCCCCC(=O)ON1C(=O)CCC1=O)C(=O)OC(C)(C)C)C(=O)OC(C)(C)C ; NCCCC[C@@H](NC(=O)[C@@H](Cc1ccccc1)NC(=O)[C@@H](Cc1ccc(O)c(I)c1)NC(=O)CC[C@H](C(=O)O)N1CCN(CC(=O)O)CCN(CC(=O)O)CC1)C(=O)O ; O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CC[C@H](C(=O)O)N1CCN(CC(=O)O)CCN(CC(=O)O)CC1)C(=O)O)C(=O)O)C(=O)O]

NODAGA-(I-y) fk (Sub-KuE) (6): To a solution of 5 (15 μg, 18 μmol, 1 eq) and TEA (13 μL, 90 μmol, 5 eq) dissolved in 600 μL of DMF was slowly added 13 μg of 4 (18 μmol, 1 eq) dissolved in 400 μL of DMF. After stirring for 2 h at RT, the reaction mixture was evaporated to dryness. Subsequent removal of tBu-protecting groups was carried out by dissolving the crude product in TFA and stirring for 40 min. After precipitation in diethyl ether, the crude product was dissolved in water and purified using preparative RP-HPLC (25% to 40% B in 20 min). HPLC (10% to 90% B in 15 min): tR=10.3 min; K′=5.44. Calculated monoisotopic mass for NODAGA-PSMA-I&T (C₅₉H₈₅IN₁₀O₂₁): 1396.5.

Alternatively, HPLC analysis was performed on a Waters XBridge Peptide BEH C18, 250×4.6 mm, 3.5 μm column; eluent A: water (0.1% H₃PO₄); eluent B: acetonitrile (0.1% H₃PO₄); 10% B to 90% linearly over 15 minutes at 1 mL/min; detection at 215 nm; retention time, 12.4 minutes. MALDI-TOF calc. [MH]+1397.5 m/z. Found 1397.8 m/z. Here performed in linear positive mode with cyano hydroxycinnamic acid as matrix.

The NODAGA-PSMA I&T was stored as 40 μg net aliquots as lyophilized powder at −20° C. and kept at 25° C. for up to 14 days showing no signs of degradation.

### 5.2.1. Production of [⁶¹Cu]Cu-NODAGA-PSMA I&T

[⁶¹Cu]Cu-NODAGA-PSMA I&T is obtained by radiolabeling the NODAGA-PSMA I&T precursor with [⁶¹Cu]Cu²⁺ in the form of [⁶¹Cu]CuCl₂. The components used for the manufacturing of the [⁶¹Cu]Cu-NODAGA-PSMA I&T solution for clinical evaluation and their respective amount and function are listed in Table 18.

TABLE 18
Components used for the manufacturing of the [⁶¹Cu]Cu-NODAGA-PSMA
I&T solution for clinical evaluation and respective function.

| Component | Volume | Function |
|---|---|---|
| [⁶¹Cu]CuCl₂ in 0.05M | 3 mL containing from | Radionuclide used for radiolabeling |
| hydrochloric acid | 0.700 to 1.050 GBq | |
| 40 μg of NODAGA-PSMA I&T | 6 mL | NODAGA-PSMA I&T is the |
| in 0.5M sodium acetate + 20 | | precursor that is radiolabeled with |
| μg/mL of ascorbic acid | | ⁶¹Cu. The solution of sodium |
| | | acetate serves to maintain a |
| | | constant pH level, and with the |
| | | addition of ascorbic acid, the |
| | | degradation of the precursor is |
| | | prevented. |
| Saline solution (0.9% sodium | 4 mL | Dilution |
| chloride) | | |

The labelling process was performed on a GE Healthcare FASTlab 2 module. An aliquot of lyophilised NODAGA-PSMA I&T (40 μg, 28 nmol) was dissolved in up to 6 mL 0.5 M sodium acetate (pH 8) containing 20 μg/mL ascorbic acid and transferred to a reaction vial. Then, 3 mL of [⁶¹Cu]CuCl₂ in 0.05 M hydrochloric acid (0.4-1.4 GBq/mL) was added to the precursor solution, reaching a pH between 5 and 7. No further purification step was necessary. The solution obtained was dispensed in an aseptic environment (ISO class 5) into the product vial (20 mL sterile evacuated vial) over a sterile Cathivex-GV 25 mm PVDF 0.22 μm filter. Saline solution for injection was added through the sterile filter, achieving a final volume of approximately 13 mL.

Two aliquots were removed by manual dispensing using a sterile syringe. Considering a potential solution loss up to 1 mL in the sterile filter, approximately 10 mL of [⁶¹Cu]Cu-NODAGA-PSMA I&T solution for injection was present at the end of the dispensing operations inside the glass vial. The composition of this solution is displayed in Table 19.

TABLE 19
Composition of the [⁶¹Cu]Cu-NODAGA-PSMA I&T solution for injection.

| | Amount per | | Amount | | |
|---|---|---|---|---|---|
| Component | batch | | per mL | | Function |
| [⁶¹Cu]Cu-NODAGA-PSMA I&T | 18-43 mCi | | 1.5-3.0 mCi/mL | | Active ingredient |
| | (660-1591 MBq | | (54-111 MBq/mL) | | |
| | at EOS) | | | | |
| L-Ascorbic acid, sodium salt | 108-133 | μg | 8.3-10.2 | μg/mL | Scavenger |
| Hydrochloric acid | 2.7-11 | mg | 0.21-0.84 | mg/mL | Component of Cu- |
| | | | | | 61 solution |
| Sodium acetate | 246 | mg | 18.92 | mg/mL | Prevent pH changes |
| | | | | | and degradation |
| Sodium chloride | 36 | mg | 2.77 | mg/mL | Component of the |
| | | | | | saline solution used |
| | | | | | for dilution |
| UltraPur Water | 9 | mL | 0.69 | mL | |

| | |
|---|---|
| Total volume | 13 ± 1.3 mL |

The release of the [⁶¹Cu]Cu-NODAGA-PSMA I&T solution for injection for clinical use is in compliance with the QC specifications listed in Table 20. All the chemicals used are trace metal grade. After manufacturing, the radiopharmaceutical can be stored at room temperature for up to 12 hours before injection.

TABLE 20
Specifications of [⁶¹Cu]Cu-NODAGA-PSMA I&T solution for clinical evaluation.

| Parameter | Test Method | Specification | |
|---|---|---|---|
| Appearance | Visual inspection | Clear, colourless solution, free from | |
| | | particulate matter | |
| Activity concentration (EoP) | Dose calibrator and | 54-111 MBq/mL or 1.5-3.0 | |
| | calculation | mCi/mL | |
| pH | pH strips | 5-7 | |
| Radiochemical Identity [⁶¹Cu]Cu- | UV-HPLC | RRT 0.9 to 1.1 | |
| NODAGA PSMA I&T | | | |
| Radiochemical purity [⁶¹Cu]Cu- | Radio-TLC | ≥95.0% | |
| NODAGA PSMA I&T | Radio-HPLC | ≥95.0% | |
| Stability*^{#} | Radio-HPLC | ≥95.0% up to 12 hours | |
| Radionuclidic identity | γ-Spectrometry | γ-photons with energy peaks at: | |
| | | 283 keV ± 20 keV | |
| | | 511 ± 20 keV (eventually sum peak | |
| | | at 1022 keV ± 20 keV) | |
| | | 656 keV ± 20 keV | |
| | Half-life via dose | 200 ± 20 | min |
| | calibrator | | |
| Radionuclidic purity* [⁶¹Cu]Cu | γ-Spectrometry | ≥99.9% | |
| | (HPGe detector) | | |
| Radionuclidic purity | γ-Spectrometry | ≤0.1% | |
| [^{56, 57, 58, 60}Co]Co* | (HPGe detector) | | |
| Bacterial endotoxin content | LAL test | ≤17.5 | EU/mL |
| Sterile filter integrity | Bubble point test | ≥46 | psi |

| | | |
|---|---|---|
| Sterility* | Sterility test | No microbial growth |

*post-release
^{#}measured periodically

[⁶¹Cu]Cu-NODAGA-PSMA I&T is diluted with saline solution to a circa 200 MBq dose for each patient, to be administered intravenously The formulation of the dose per patient is provided in Table 21.

TABLE 21
Components of the [⁶¹Cu]Cu-NODAGA-PSMA I&T solution for injection.

| Component | Amount | | Concentration | Function |
|---|---|---|---|---|
| [⁶¹Cu]Cu-NODAGA-PSMA | 200 ± 50 | MBq | 10-25 MBq/mL | Active ingredient |
| I&T | | | | |
| Radiolabeling solution^{a} | 2.40-5.50 | mL | 25-55% (v/v) | Prevent pH changes |
| | | | | and degradation |
| Saline solution (0.9% | 4.50-7.60 | mL | 45-75% (v/v) | Dilution |

sodium chloride)

^{a}The composition of the radiolabeling solution is 46% 0.5M sodium acetate ± 20 μg/mL ascorbic acid, 23% 0.05M hydrochloric acid and 31% saline solution.

### 5.2.2. Shelf Life

The shelf life of [⁶¹Cu]Cu-NODAGA-PSMA I&T was assessed at room temperature in the reaction solution and the injection formulation at various time points after EoS. At pre-defined time points (up to 12 h), an aliquot was analyzed by radio-HPLC. The radiochemical purity was determined by analytical reverse-phase radio-HPLC using a Macherey Nagel Nucleodur ISIS C18 (5 μm, 250×4.6 mm) column. The results indicated a stability of over 95% up to 12 hours (Table 22).

TABLE 22
[⁶¹Cu]Cu-NODAGA-PSMA I&T shelf-life
determination by reverse-phase radio-HPLC.

| | RCP | up to 3 h | up to 6 h | up to 9 h | up to 12 h |
|---|---|---|---|---|---|
| | by HPLC | (n = 3) | (n = 3) | (n = 3) | (n = 2) |
| Reaction | 99.4 ± 0.4% | 99.6 ± 0.2% | 99.5 ± 0.7% | 99.1 ± 0.8% | 99.7 ± 0.1% |
| solution | | | | | |
| Injection | 99.0 ± 0.2% | 99.5 ± 0.2% | 99.0 ± 0.5% | 99.1 ± 0.5% | 99.7 ± 0.41% |
| formulation^{a} | | | | | |

^{a}Formulation as reported in Table 20.

### 5.2.3. Lipophilicity (log D (pH 7.4))

The lipophilic/hydrophilic character of [⁶¹Cu]Cu-NODAGA-PSMA I&T was assessed by the determination of the distribution coefficient (D), expressed as log D at physiological pH (log D (pH 7.4)), between an aqueous and an organic phase following the “shake-flask” method. [⁶¹Cu]Cu-NODAGA-PSMA I&T (1 μM) was added to a 50:50 pre-saturated mixture solution of 1-octanol and phosphate-buffered saline (PBS pH 7.4). The mixture was vortexed for 30 min and then centrifuged at 3000 rpm to achieve phase separation. Aliquots from each phase were collected and measured in a gamma counter. The distribution coefficient was calculated as the average logarithmic values of the ratio between the radioactivity in the organic and the PBS phase. Calculations revealed that log D (pH 7.4)=−2.95±0.08. This value does not differ from the mean of other compounds in the small-molecule PSMA inhibitor class. It indicates that the molecule tends to partition into aqueous body compartments and excreted through the kidneys.

### 5.3. Example 3: In Vitro and In Vivo Studies

### 5.3.1. Binding Affinity (IC₅₀)

The binding affinity of NODAGA-PSMA I&T was determined by measuring IC₅₀ by labelling the precursor with natural copper (^{nat}Cu). The IC₅₀ value is the concentration in which 50% of the specific binding of a reference radioligand to the tested molecule is inhibited. The radioiodinated ((S)-1-carboxy-5-(4-(-¹²⁵I-iodo-benzamido) pentyl) carbamoyl)-L-glutamic acid ([¹²⁵I]I-BAKuE) was used as reference radioligand. The assay was performed on LNCaP cells, human prostate cancer cells characterized by high expression levels of PSMA.

^{nat}Cu-NODAGA-PSMA I&T was produced by dissolving NODAGA-PSMA I&T (0.7 μmol) in 0.30 mL of ammonium acetate (0.5 M, pH 8), followed by the addition of 0.15 mL CuCl₂ in 0.05 M hydrochloric acid. The reaction mixture was incubated for 15 min at 95° C. The product was purified from unlabeled ^{nat}Cu using a SepPak Classic C18 cartridge (Waters, Eschborn, Germany), washed with water, and eluted with 2 mL methanol. After evaporation of methanol, the product was reconstituted in 0.3 mg/mL 30% (v/v) acetonitrile in water.

The assay was performed on LNCaP cells seeded in 24-well plates. The cells were incubated with increasing concentrations of ^{nat}Cu-NODAGA-PSMA I&T (ranging from 0.1 up to 100 nM) in the presence of 0.2 nM [¹²⁵]I-BAKuE. After 1 hour of incubation on ice, the unbound (free) [¹²⁵I]I-BAKuE was collected by removing the medium (EMEM EBS) and the cells were detached with 1 M sodium hydroxide for counting (bound radioligand). Non-specific binding was defined as the amount of binding activity in high excess (10 μM) of the blocking agent, 2-(phosphonomethyl) pentanedioic acid (2-PMPA). The measured affinity was IC₅₀=9.3±1.8 nM. The value obtained aligns with those reported for radiopharmaceuticals of the same family. The high IC₅₀ value results in increased specificity of the investigated radiopharmaceutical to PSMA, minimizing uptake in non-targeted tissues. It also ensures adequate retention in targeted tissues, allowing enhanced contrast at delayed time points.

### 5.3.2. In Vitro Cellular Uptake, Internalization and Dissociation

The cellular uptake of [⁶¹Cu]Cu-NODAGA-PSMA I&T was studied in vitro using intact LNCaP cells seeded overnight in 6-well plates. On the day of the experiment, the cells were washed and incubated with [⁶¹Cu]Cu-NODAGA-PSMA I&T at different time points, either alone or in the presence of 2-PMPA (10 μM) as a blocking agent, to distinguish between specific and non-specific uptake. At each investigated time point, the medium containing the unbound (free) [⁶¹Cu]Cu-NODAGA-PSMA I&T was removed, followed by two washing steps with ice-cold phosphate-buffered saline. The cells were then treated for 2×5 min with ice-cold glycine solution (0.05 M, pH 2.8) to detach the cell surface-bound tracer (acid released). Afterwards, the cells containing the internalized [⁶¹Cu]Cu-NODAGA-PSMA I&T were detached with 1 M sodium hydroxide at 37° C. and collected for measurement. The specific cell surface-bound and internalized tracer was expressed as a percentage of the total applied activity after subtracting the non-specific values. The results are presented in Table 23 as a mean value (n=3±standard deviation).

The results demonstrate that the highest accumulation on the cell membrane occurred within 30 minutes (saturation of binding sites), while the radiotracer was continuously internalized for up to 120 minutes. These results align with those of other radiopharmaceuticals from the same class. The binding to PSMA results in the formation of a complex internalized into the cell through clathrin-mediated endocytosis, which provides an extended retention time in the target tissues in vivo.

TABLE 23
Total cellular uptake of [⁶¹Cu]Cu-NODAGA-PSMA
I&T distributed between the cell surface (cell
membrane-bound) and the internalized fractions.

| Time point | Cell surface | Internalised | Total uptake |
|---|---|---|---|
| [min] | fraction [%] | fraction [%] | [%] |
| 5 | 4.3 ± 0.7 | 1.2 ± 0.2 | 5.5 ± 0.9 |
| 15 | 8.2 ± 1.0 | 3.9 ± 0.4 | 12.1 ± 1.4 |
| 30 | 10.7 ± 1.0 | 7.0 ± 1.1 | 17.7 ± 2.1 |
| 60 | 10.8 ± 1.8 | 11.7 ± 1.6 | 22.5 ± 3.4 |
| 120 | 10.2 ± 1.8 | 17.6 ± 2.8 | 27.8 ± 4.6 |

### 5.3.3. In Vitro Efflux Rate

Efflux-rate assays were performed on 6-well plates pre-treated with poly-L-lysine to promote adhesion. The plated cells were incubated with [⁶¹Cu]Cu-NODAGA-PSMA I&T (100 μL, 0.5 nM) at 37° C. For non-specific uptake measurement, 100 μL of 2-PMPA (10 μM) was added to some wells. After 1 h, the cell-released fraction was collected by removing the medium and washing it twice with PBS. Then, the cells were incubated at 37° C. in a fresh medium for up to 180 min. At each time point, the medium was removed and replaced by fresh pre-warmed (37° C.) medium. At the end of the experiment, the cells were detached with 1 M sodium hydroxide and collected to determine the remaining cell-binding [⁶¹Cu]Cu-NODAGA-PSMA I&T. The different fractions were quantified using a γ-counter (Cobra II, Packard A Canberra) and represented as a percentage of the applied radioactivity.

The experiment was also performed in the presence of a competitor (100-fold excess DOTAGA-PSMA I&T) to obtain a “rebinding free” system. The presence of the competitor prevented any rebinding of [⁶¹Cu]Cu-NODAGA-PSMA I&T that dissociates faster. The values were calculated as a percentage of the cell-associated fraction (surface-bound and internalized fraction), subtracting the non-specific uptake (measured in the presence of 10 μM 2-PMPA). The results are presented in Table 24.

The slow efflux rate suggests that the investigated radiopharmaceutical will have a prolonged residence time within the tumor cells, which can impact the total uptake and signal recorded by the PET instrument at later time points. The effect of the rebinding is prominent due to the high binding affinity.

TABLE 24
Efflux kinetics of [⁶¹Cu]Cu-
NODAGA-PSMA I&T in LNCaP cells.

| | |
|---|---|
| Time point | Cell-associated fraction [%] |

| [min] | Without competitor | With competitor |
|---|---|---|
| 10 | 83.8 ± 0.7 | 67.9 ± 1.1 |
| 20 | 73.5 ± 0.8 | 54.4 ± 0.7 |
| 30 | 65.2 ± 1.4 | 46.5 ± 0.4 |
| 45 | 57.0 ± 2.8 | 39.9 ± 0.6 |
| 60 | 51.3 ± 3.3 | 35.9 ± 0.9 |
| 90 | 45.0 ± 3.3 | 29.9 ± 1.1 |
| 120 | 40.0 ± 3.5 | 25.0 ± 0.5 |
| 150 | 35.4 ± 3.1 | 20.9 ± 0.4 |
| 180 | 31.5 ± 2.5 | 17.5 ± 0.5 |

### 5.3.4. Pharmacokinetics and Product Metabolism in Mice

Unless otherwise specified, the in vivo studies were performed on athymic nude Foxn1^{nu} 4-6-week-old mice. Animals were injected in the shoulder with LNCaP cells (10⁷ cells/200 μL) suspended in 1:1 (v: v) EMEM EBS medium and Matrigel. The in vivo experiments were performed when tumors reached a volume between 100-500 mm³. At the intervention, all mice were between 7 and 11 weeks old. All animal experiments were performed following the guidelines for the use of living animals in scientific studies and the Swiss regulations for the protection of animals.

### 5.3.5. Biodistribution Studies

Quantitative biodistribution studies were performed on LNCaP xenografted mice to determine the distribution of [⁶¹Cu]Cu-NODAGA-PSMA I&T following i.v. injection into the tail vein. The dose of the administered radiopharmaceutical was between 1.5-3.5 MBq (200 μmol) diluted to 100 μL with saline solution. The mice were divided into two groups of eight (5 males and 3 females) and euthanized at 1- and 4-hours post-injection. The organs of interest were then collected, rinsed, blotted, weighed, and analyzed in a gamma counter. The results were expressed as the percentage of injected activity per gram of tissue (% IA/g) and reported as mean±standard deviation in Table 25. The tumor-to-normal organ ratios are reported in Table 26.

The highest uptake was observed in the kidneys (91±10% IA/g at 4 hours), followed by the LNCaP tumor (10.7±3.3% IA/g at 4 hours) and adrenal glands (8.32±2.87% IA/g at 4 hours), while the lowest activity was observed in the blood (0.10±0.03% IA/g at 4 hours). These results suggest that [⁶¹Cu]Cu-NODAGA-PSMA I&T has a fast clearance from the bloodstream and is rapidly excreted through the kidneys. This phenomenon was further investigated in the pharmacokinetic studies in healthy mice.

The high activity observed in the kidneys, adrenal glands, salivary glands, and LNCaP-tumor indicates specific binding by these tissues in line with PSMA expression (Silver, D. A., Pellicer, I., Fair, W. R., Heston, W. D. W. & Cordon-Cardo2, C. Prostate-specific membrane antigen expression in normal and malignant human tissues. AACR 3, 81-85). The specificity was further investigated by specificity studies. The general decrease in % IA/g at 4 hours post-injection suggests a gradual elimination of the radiopharmaceutical from all the body compartments, potentially due to the efflux rate or metabolic degradation. Despite that, the washout rate was less pronounced in tumors than in most of the other tissues, leading to an increase in the tumor-to-organ uptake ratio. At 4 hours post-injection, the tumor-to-blood ratio increased from 50 to 107, and the tumor-to-muscle ratio increased from 13 to 21. These results suggest that at 4 hours post-injection, the PET-imaging contrast increased significantly compared with the 1-hour time point. The improved contrast allows the visualization of lesions masked by the background signal, leading to an improved metastasis detection rate. Salivary glands showed a marked decrease in accumulated activity (2.01±0.33% IA/g at 1 hour vs 0.52±0.06% IA/g at 4 hours), indicating a quick efflux rate and a reduced burden for the patients in this PSMA-expressing tissue.

TABLE 25
Biodistribution of [⁶¹Cu]Cu-NODAGA-PSMA I&T in LNCaP
xenografted female mice at 1 and 4 hours post-injection.
Results are expressed as % IA/g ± standard deviation.

| Organ | 1 hour | 4 hours |
|---|---|---|
| Blood | 0.28 ± 0.06 | 0.10 ± 0.03 |
| Heart | 0.45 ± 0.15 | 0.22 ± 0.05 |
| Lung | 1.69 ± 0.45 | 0.74 ± 0.19 |
| Liver | 1.02 ± 0.28 | 0.72 ± 0.11 |
| Pancreas | 0.97 ± 0.36 | 0.45 ± 0.09 |
| Spleen | 6.04 ± 1.87 | 1.28 ± 0.39 |
| Stomach | 1.13 ± 0.20 | 0.66 ± 0.15 |
| Intestine | 2.11 ± 0.78 | 1.06 ± 0.50 |
| Adrenal | 17.3 ± 3.26 | 8.32 ± 2.87 |
| Kidneys | 118 ± 13 | 91 ± 10 |
| Muscle | 1.12 ± 0.32 | 0.50 ± 0.22 |
| Bone | 2.73 ± 0.91 | 1.34 ± 0.48 |
| Salivary glands | 2.01 ± 0.33 | 0.52 ± 0.06 |
| LNCaP-tumor | 14.0 ± 5.0 | 10.7 ± 3.3 |

TABLE 26
Tumor-to-organ uptake ratios of [⁶¹Cu]Cu-NODAGA-PSMA I&T
in LNCaP xenografted female mice at 1 and 4 hours post-injection.

Time Points

| Tumor/Organ Ratios | 1 hour | 4 hours |
|---|---|---|
| Tumor/Blood | 50 | 107 |
| Tumor/Liver | 14 | 15 |
| Tumor/Kidney | 0.12 | 0.12 |
| Tumor/Salivary gland | 7 | 21 |
| Tumor/Muscles | 13 | 21 |

### 5.3.6. Pharmacokinetics in Healthy Mice

The pharmacokinetics of the investigated radiopharmaceutical was evaluated in healthy female BALB/c mice from 1 to 24 hours. For the latest time points (12 and 24 hours), NODAGA-PSMA I&T was radiolabeled with ⁶⁴Cu obtained from the Tübingen University Hospital. The use of a different PET copper isotope is justified by the longer half-life of ⁶⁴Cu (t_{1/2}=12.7 hours), which is more appropriate for tests that include delayed time points. Additionally, using an isotope from the same element ensures that the radiopharmaceutical retains its chemical and physical properties, guaranteeing consistency in the study. Mice were injected i.v. with 4 MBq (200 pmol) of [^{61/64}Cu]Cu-NODAGA-PSMA I&T diluted to 100 μL with saline solution. The data were combined with the results obtained from the groups of xenografts at 1 and 4 hours post-injection, as the biodistribution in nude mice was the same as in the healthy mice. The results are presented in Table 27 and are expressed as mean±standard deviation.

In vivo, the radiotracer undergoes rapid renal excretion due to the high hydrophilicity and tendency to not interact with blood proteins. The accumulated activity in the kidneys continued to decrease, reaching values<20% IA/g at 24 hours post-injection. The hepatic excretion route, often associated with the presence of free copper isotopes (Kjærgaard, K. et al. Intravenous and oral copper kinetics, biodistribution and dosimetry in healthy humans studied by [64Cu]copper PET/CT. EJNMMI Radiopharm Chem 5, (2020)), is marginal, as proven by the low background in the liver. A considerable uptake of [^{61/64}Cu]Cu-NODAGA-PSMA I&T was also observed in the spleen. The higher uptake is primarily attributed to the expression of PSMA in some splenic regions. In addition, the spleen acts as a filter for immune cells and cell debris, which may result in the accumulation of the PSMA ligand tracer in this organ.

In conclusion, the rapid excretion of the radiopharmaceutical through the kidneys minimizes the risk of radioactive dose accumulation in patients, assuring the safety of the imaging procedures. The investigated radiopharmaceutical showed a consistent washout from all healthy organs, including those which expressed PSMA, except for the kidneys.

The findings were in accordance with previously reported results for radiopharmaceuticals in the same class (van der Gaag, S. et al. Pharmacological Optimization of PSMA-Based Radioligand Therapy. Biomedicines 10, (2022)).

TABLE 27
Biodistribution data on healthy mice of [^{61/64}Cu]Cu-NODAGA-PSMA
I&T at 1, 4, 12 and 24 hours post-injection. Values are
presented as the mean of % AI/g ± standard deviation.

| Organ | 1 hour* | 4 hours^{#} | 12 hours^{¥} | 24 hours^{¥} |
|---|---|---|---|---|
| Blood | 0.33 ± 0.09 | 0.11 ± 0.03 | 0.11 ± 0.01 | 0.08 ± 0.01 |
| Heart | 0.49 ± 0.12 | 0.25 ± 0.06 | 0.26 ± 0.05 | 0.21 ± 0.02 |
| Lung | 1.49 ± 0.45 | 0.68 ± 0.22 | 0.52 ± 0.11 | 0.32 ± 0.12 |
| Liver | 1.12 ± 0.25 | 0.88 ± 0.25 | 0.96 ± 0.18 | 0.84 ± 0.11 |
| Pancreas | 1.37 ± 0.90 | 0.55 ± 0.16 | 0.28 ± 0.04 | 0.17 ± 0.03 |
| Spleen | 9.33 ± 3.81 | 2.35 ± 1.45 | 1.40 ± 0.57 | 0.58 ± 0.15 |
| Stomach | 1.12 ± 0.15 | 0.72 ± 0.15 | 0.52 ± 0.03 | 0.31 ± 0.05 |
| Intestine | 2.11 ± 0.85 | 1.12 ± 0.48 | 0.94 ± 0.41 | 0.46 ± 0.11 |
| Adrenal | 14.38 ± 4.39 | 7.31 ± 2.76 | 2.99 ± 0.93 | 1.17 ± 0.22 |
| Kidneys | 124 ± 21 | 94 ± 12 | 60 ± 9 | 16.1 ± 4.9 |
| Muscles | 0.99 ± 0.30 | 0.45 ± 0.18 | 0.25 ± 0.05 | 0.08 ± 0.02 |
| Femur | 2.48 ± 0.97 | 1.34 ± 0.38 | 0.50 ± 0.10 | 0.20 ± 0.05 |
| Salivary | 1.89 ± 0.28 | 0.58 ± 0.12 | 0.39 ± 0.05 | 0.22 ± 0.05 |
| glands | | | | |

*n = 16,
^{#}n = 13,
^{¥}n = 5

### 5.3.7. PET/CT Imaging in Mice

LNCap xenografted mice were injected i.v. into the tail vein with 4-8 MBq (400 pmol) of [⁶¹Cu]Cu-NODAGA-PSMA I&T diluted in 100 μL saline solution. Dynamic PET scans were acquired for 1 hour upon injection of the radiotracer. The mice were euthanized using carbon dioxide at 4 hours post-injection, the bladder was mechanically emptied, and static PET scans were acquired for 30 min. PET images were obtained using the β-CUBE PET scanner system (MOLECUBES, Gent, Belgium) and were decay corrected and reconstructed with the VivoQuant software version 4.0. Tomograms and helical CT scans of the whole mouse were first acquired using a NanoSPECT/CTTM scanner (Bioscan Inc.). CT images were reconstructed using CTReco (version r1.146), resulting in a pixel size of 0.2 mm. InVivoScope (version 1.43, Bioscan Inc.) software was used to generate the maximum intensity projection (MIP) of the co-registered PET/CT images presented in FIG. 9.

The images show the quick accumulation of [⁶¹Cu]Cu-NODAGA-PSMA I&T in the tumor, with a maximum reached at 1 hour post-injection. The constant washout of [⁶¹Cu]Cu-NODAGA-PSMA I&T from off-target body compartments leads to a consistent decrease of the background signal, particularly evident in the dynamic scan. The optimal tumor-to-background signal ratio was reached in the scan acquired 4 hours post-injection. The results demonstrated the feasibility of using [⁶¹Cu]Cu-NODAGA-PSMA I&T for delayed time point imaging to enhance contrast. This impacts the overall quality of the PET image, allowing for the visualization of malignant lesions that may have gone unnoticed with traditional PSMA-PET/CT tracers. Such lesions may include (I) malignant lesions with low expression of PSMA, (II) cancerous tissues in the area of the primary tumor resection, and (III) small lymph nodes and distant metastases. The use of [⁶¹Cu]Cu-NODAGA-PSMA I&T, therefore, may increase the tumor detection ratio.

### 5.3.8. Specificity Studies

The specificity of [⁶¹Cu]Cu-NODAGA-PSMA I&T for PSMA-expressing tissues was evaluated in LNCaP xenograft mice through a blocking study. The mice were first administered with 1.3 μmol (300 μg) of 2-PMPA as a blocking agent, followed by an intravenous injection of 4-8 MBq (400 μmol) [⁶¹Cu]Cu-NODAGA-PSMA I&T diluted to 100 μL in saline in the tail vein. The animals (n=4) were sacrificed 1 hour post-injection, and the organs were collected, weighed and counted in a gamma counter as already described. The results are expressed as a percentage of injected activity per gram (% IA/g) as presented in Table 27 and are compared with the results obtained from biodistribution studies.

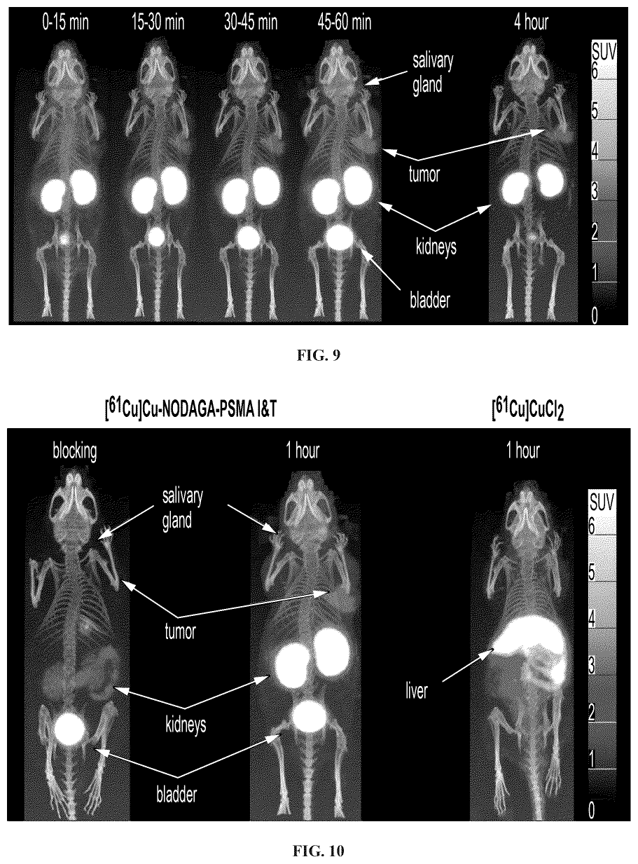

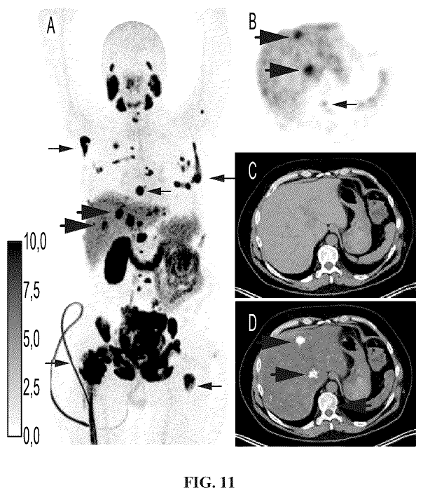

PET/CT imaging was also performed at 1 hour post-injection. Additionally, a PET/CT imaging study was conducted in a xenografted mouse after injection of 7 MBq [⁶¹Cu]CuCl₂ (in 100 μL saline solution) to evaluate the total body distribution of non-complexed ⁶¹Cu. The biodistribution results are reported in Table 28. The imaging results are presented in FIG. 10.

The most significant blocking effect was seen in the tumor (14.0±5.0 vs 0.68±0.54% IA/g) as well as in all the PSMA-expressing normal organs such as kidneys (118±13 vs 4.59±0.50% IA/g), adrenal glands (17.3±3.26 vs 0.96±0.87% IA/g), spleen (6.04±1.87 vs 0.33±0.16% IA/g), salivary glands, and intestine. The significant blocking effect in PSMA-expressing organs proves that the high affinity for the biomarker drives the binding in these tissues typical of the pharmaceutical class to which [⁶¹Cu]Cu-NODAGA-PSMA I&T belongs. High selectivity ensures a reduced dose load for the body's off-target compartments, warranting enhanced patient safety.

The residual uptake in the kidneys confirmed that the investigated radiopharmaceutical was excreted primarily through the kidneys. Since the larger part of the uptake was due to specific binding, the co-administration of an agent that would enhance renal excretion, such as gelofusine, cannot efficiently reduce the accumulation. Therefore, kidney accumulation is a concern as it may lead to a potential radioactive dose load for patients. Absorbed organ doses were further investigated by dosimetry calculations.

Liver accumulation remained stable at low levels, indicating that only a small portion of the radiopharmaceutical metabolites were excreted through the liver. The PET/CT imaging performed after [⁶¹Cu]CuCl₂ injection confirmed that free isotopes were excreted mainly through the liver, due to the coordination by blood proteins metabolized through the liver as ceruloplasmin, metallothionein and Copper Transport Regulator 1 (CTR1). In general, therefore, the investigated radiopharmaceutical is stable in vivo, with limited transchelation phenomena.

TABLE 28
Specificity of [⁶¹Cu]Cu-NODAGA-PSMA I&T in LNCaP
xenografts at 1-hour post-injection. Results are expressed
as the mean of the % IA/g ± standard deviation.

| Organ | 1 hour* | 1 hour blocking^{#} |
|---|---|---|
| Blood | 0.28 ± 0.06 | 0.44 ± 0.50 |
| Heart | 0.45 ± 0.15 | 0.34 ± 0.27 |
| Lung | 1.69 ± 0.45 | 0.67 ± 0.40 |
| Liver | 1.02 ± 0.28 | 1.29 ± 0.53 |
| Pancreas | 0.97 ± 0.36 | 0.29 ± 0.19 |
| Spleen | 6.04 ± 1.87 | 0.33 ± 0.16 |
| Stomach | 1.13 ± 0.20 | 0.62 ± 0.28 |
| Intestine | 2.11 ± 0.78 | 0.87 ± 0.50 |
| Adrenal | 17.3 ± 3.26 | 0.96 ± 0.87 |
| Kidneys | 118 ± 13 | 4.59 ± 0.50 |
| Muscle | 1.12 ± 0.32 | 0.32 ± 0.36 |
| Bone | 2.73 ± 0.91 | 0.39 ± 0.24 |
| Salivary glands | 2.01 ± 0.33 | 0.51 ± 0.30 |
| LNCaP-tumor | 14.0 ± 5.0 | 0.68 ± 0.54 |

*n = 16,
^{#}n = 4.

### 5.3.9. Dosimetry Calculations

The data from the pharmacokinetic studies (Table 26) was decay-corrected using the half-life of ⁶¹Cu to extrapolate the human dose. Since there were no measurements of the radioactivity accumulation in red marrow, this was estimated assuming a linear relationship between the blood and the red marrow residence times. The proportionality factor used was the ratio between the red marrow mass and human blood mass. The residence time (RT) for the stomach was approximated to be equal to the RT of the stomach wall since the animals were subjected to fasting before the biodistribution experiments. The heart was emptied from blood before measurements, and it was assumed that the total measured heart uptake was associated with the heart wall.

OLINDA/EXM 1.0 was used to integrate the fitted time-activity curves and to estimate the organ and effective doses using standard human whole-body adult female and male phantom models. Since the salivary glands are PSMA-positive and the organ is not included in the phantom, the dose absorbed was calculated separately, assuming that the weight of the human salivary gland is 30 g. The results are summarized in Table 29.

The kidneys received the highest absorbed dose (1.00E+00 mGy/MBq for females and 1.74E+00 mGy/MBq for males), followed by the spleen, the pancreas, and the small intestine. The absorbed dose by the other organs was relatively low, with the brain and skin having the lowest values. Despite being higher than other organs, the absorbed dose in the kidneys after injecting 100-150 MBq [⁶¹Cu]Cu-NODAGA-PSMA I&T would be approximately 0.17-0.26 Gy, hundreds-fold lower than the currently accepted critical threshold of kidney absorbed dose of 23 Gy (Cremonesi, M., Ferrari, M., Bodei, L., Tosi, G. & Paganelli, G. Dosimetry in Peptide Radionuclide Receptor Therapy: A Review*. J Nucl Med 47, 1467-1475 (2006)).

An effective dose, which is an estimation of the overall radiation exposure to the whole body, was 0.0095 mSv/MBq for females and 0.0142 mSv/MBq for males. In comparison, the approved pharmaceutical PET radiotracers [¹⁸F]F-DCFPyL and [⁶⁸Ga]Ga-PSMA-11 show an effective dose of 0.017 mSv/MBq and 0.023 mSv/MBq, respectively. PET imaging with [⁶¹Cu]Cu-NODAGA-PSMA I&T would result in a lower radiation dose to the patient than the most clinically established PSMA-targeting PET radiotracers.

Regarding gender differences, it has been noted that the absorbed dose was generally higher in males than in females. This difference may be due to differences in body composition, such as organ size and tissue density, which can affect the distribution and clearance of the radiotracer. Overall, the calculated radiation doses are considered suitable for application in humans.

TABLE 29
Total absorbed doses in different
organs of [⁶¹Cu]Cu-NODAGA-PSMA I&T.

Total absorbed doses
[mGy/MBq]

| Target organ | Female | Male |
|---|---|---|
| Adrenals | 9.86E−02 | 8.39E−02 |
| Brain | 5.59E−05 | 6.18E−05 |
| Breasts | 1.23E−03 | — |
| Gallbladder Wall | 1.37E−02 | 2.30E−02 |
| LLI Wall | 3.24E−03 | 4.64E−03 |
| Small Intestine | 2.78E−02 | 3.79E−02 |
| Stomach Wall | 1.08E−02 | 1.71E−02 |
| ULI Wall | 9.78E−03 | 1.43E−02 |
| Heart Wall | 5.03E−03 | 6.94E−03 |
| Kidneys | 1.00E+00 | 1.74E+00 |
| Liver | 1.83E−02 | 2.51E−02 |
| Lungs | 4.86E−03 | 6.11E−03 |
| Muscle | 4.03E−03 | 6.00E−03 |
| Ovaries | 4.76E−03 | — |
| Pancreas | 3.18E−02 | 4.83E−02 |
| Red Marrow | 6.94E−03 | 1.11E−02 |
| Osteogenic Cells | 3.93E−03 | 5.31E−03 |
| Skin | 1.82E−03 | 2.90E−03 |
| Spleen | 5.65E−02 | 7.37E−02 |
| Testes | — | 5.39E−04 |
| Thymus | 1.11E−03 | 1.67E−03 |
| Thyroid | 2.72E−04 | 4.99E−04 |
| Urinary Bladder Wall | 1.60E−03 | 2.06E−03 |
| Uterus | 3.94E−03 | — |
| Total Body | 9.46E−03 | 1.38E−02 |
| Effective Dose [mSv/MBq] | 9.54E−03 | 1.42E−02 |

5.3.10. Toxicology—Non-clinical safety studies

The vector-induced toxicity was evaluated using the non-radiolabeled precursor in line with the IAEA and EMA guidelines (European Medicines Agency. Guideline on the non-clinical requirements for radiopharmaceuticals. (2018)); (International Atomic Energy Agency. Guidance for Preclinical Studies with Radiopharmaceuticals. (2023)). The non-clinical safety study was performed using the microdose approach I trial described in the ICH guideline M3 (R2) (European Medicines Agency. ICH guideline M3 (R2) on non-clinical safety studies for the conduct of human clinical trials and marketing authorization for pharmaceuticals. (2009)).

The study was designed as an extended single-dose toxicity study in one rodent species and performed under GLP conditions. NODAGA-PSMA I&T toxicity was evaluated after intravenous administration on day 1 of the study, followed by an observation period of 2 weeks. 60 (30 males and 30 females) CD-1 mice were divided into one dose group and one control group. The test item was administered once by i.v. administration into the tail vein. In each of the two groups, (I) 10 male and 10 female animals were euthanized the day after dosing, (II) 5 male and 5 female recovery animals were kept for an observation period of 2 weeks after dosing to detect possible delayed occurrence or persistence of, or recovery from toxic effects.

The dose animals' group was injected with 5 mg/kg NODAGA-PSMA I&T in 5% dimethyl sulfoxide (application volume 5 mL/kg) as a vehicle. The control group underwent injection of the vehicle alone (5 mL/kg). The animals were observed daily for signs of toxicity and morbidity/mortality. All animals were sacrificed for necropsy. Blood was taken at the time of necropsies to analyze hematological and clinical biochemistry parameters and urine for urinalysis. The wet weight of a tissue subset was taken, and a set of organs/tissues was preserved. A complete histopathological evaluation of the tissues was performed on treated and control animals.

NODAGA-PSMA I&T was clinically well-tolerated; no treatment-related mortality or acute adverse effect was registered. No effects on body weight gain or food consumption were noted. The dose injected is associated with a decrease in platelet count, a decrease in glucose level and an increase in potassium level. All these changes were transitory and considered non-adverse. No adverse effects on organ weights were noted. No macroscopic or microscopic findings that could be attributed to treatment with the test item were observed.

The results of this study establish a no adverse effect level (NOAEL) for NODAGA-PSMA I&T at 5 mg/kg. The NOAEL is consistent with the results obtained for other precursors in the same class. The intended average dose for patients is 0.4 μg/kg, which leads, considering the dose conversion factor (Nair, A. B. & Jacob, S. A simple practice guide for dose conversion between animals and human. J Basic Clin Pharm 7, 27 (2016)), to a safety ratio of 1000 times.

### 5.4. Example 4: First-In-Human Study

A [⁶¹Cu]Cu-NODAGA-PSMA I&T dose of 104 MBq (2.81 mCi) was administered intravenously to a 48-year old patient with known metastatic prostate cancer. The patient was co-injected with 10 mg furosemide (Lasix®, Sanofi-Aventis, Frankfurt, Germany). The [⁶¹Cu]Cu-NODAGA-PSMA I&T was prepared as described in Examples 1 and 2 and had the specifications shown in Table 30.

TABLE 30
Specifications of [⁶¹Cu]Cu-NODAGA-PSMA I&T.

| Parameter | Test method | Specifications | |
|---|---|---|---|
| Appearance | Visual inspection | Colorless, clear solution | |
| Bacterial endotoxin content | LAL test | <17.5 | EU/mL |
| Free [⁶¹Cu]Cu | Radio-TLC | ≤5% | |
| pH | pH strips | 5.0-7.0 | |
| Radioactive concentration | Dose calibrator | 8-15 | MBq/mL |
| Radiochemical purity (EoS) | Radio-TLC | ≥95% | |
| | Radio-HPLC | ≥95% | |
| Radionuclidic identity | γ-Spectrometry | γ-photons with energy peaks at: | |
| | | 283 keV ± 20 keV | |
| | | 511 ± 20 keV (eventually sum | |
| | | peak at 1022 keV ± 20 keV) | |
| | | 656 keV ± 20 keV | |
| Radionuclidic purity* | γ-Spectrometry | ≤0.01% | |
| [^{56,57,58,60}Co]Co | | | |
| Radionuclidic purity* [⁶¹Cu]Cu | γ-Spectrometry | ≥99.99% | |
| Sterile filter integrity | Bubble point test | 3.0-4.5 | bar |

| | | |
|---|---|---|
| Sterility* | Sterility test | No microbial growth |

*post-release

Imaging was performed at 3 hours following radiotracer administration on a Biograph Vision 600 (Siemens, Germany) PET/CT scanner. Images were obtained from the skull to mid-thigh and reconstructed into multiplanar PT, CT, and fused PET/CT images. CT was used for attenuation correction. PET imaging results are shown in FIG. 11.

Radiotracer accumulation was noted in multifocal osseous and hepatic metastases, as well as in the expected physiologic distribution of PSMA-targeted tracers in the lacrimal glands, salivary glands, liver, spleen, kidneys, ureters, bladder, and proximal small bowel.

[⁶¹Cu]Cu-NODAGA-PSMA I&T exhibits a high affinity for PSMA and demonstrated substantial accumulation and retention within PSMA-expressing tumors, particularly in the context of prostate cancer. The washout from off-target tissues was fast. Non-clinical dosimetry and toxicology tests demonstrate that it has a sufficient safety profile. [⁶¹Cu]Cu-NODAGA-PSMA I&T enables access to a delayed time point imaging, which increases the contrast of the PET image, leading to increased diagnostic sensitivity, finally resulting in an improved detection rate of malignant lesions. This can overcome the limitations of current PET radiotracers such as [¹⁸F]F-FDG, which lacks specificity and established PSMA-PET radiotracers that have limited uptake in tumors and are radiolabeled with radionuclides that have a short half-life.

### 5.5. Example 5: Human Clinical Trial for [⁶¹Cu]Cu-NODAGA-PSMA I&T

### 5.5.1. Objective(s)

Primary study objective: demonstrate the safety of imaging with [⁶¹Cu]Cu-NODAGA-PSMA I&T.

Secondary study objective: demonstrate the effectiveness of [⁶¹Cu]Cu-NODAGA-PSMA I&T to image prostate cancer compared to a currently FDA-approved PSMA-targeting radiotracer.

### 5.5.2. Study Overview

This is a phase I, single center, non-randomized trial with 6-10 subjects. Each subject will have biopsy-proven prostate cancer visualized on FDA-approved ¹⁸F-piflufolastat PET/CT performed within 3 weeks of trial recruitment. Each subject will receive a single administration of [⁶¹Cu]Cu-NODAGA-PSMA I&T, followed by PET/CT imaging. Safety of [⁶¹Cu]Cu-NODAGA-PSMA I&T will be evaluated by monitoring for unlikely adverse effects. Ability of [⁶¹Cu]Cu-NODAGA-PSMA I&T to visualize malignant lesions will be evaluated by comparing the number of suspicious lesions demonstrated on FDA-approved ¹⁸F-piflufolastat PET/CT with the number of suspicious lesions demonstrated on the experimental [⁶¹Cu]Cu-NODAGA-PSMA I&T though 1-sided Wilcoxon signal-rank tests for paired data.

### 5.5.3. Criteria for Evaluation

Primary Endpoint: As this is a phase I trial, safety is the primary endpoint. There is no efficacy component of this trial.

Safety Evaluations: As the [⁶¹Cu]Cu-NODAGA-PSMA I&T is given at a low, imaging dose, serious adverse events are not expected. Minor side effects that are infrequently seen with known PSMA-targeting PET imaging agents include pain at the administration site and dysgeusia. Subjects will be monitored during and for 2 hours after tracer administration, and a follow-up phone call the following day will be performed to evaluate for rare side effects.

### 5.5.4. Subject Selection

Subjects with biopsy-proven prostate cancer who meet the inclusion and exclusion criteria will be eligible for participation in this study.

### Inclusion Criteria:

1. Biopsy proven prostate adenocarcinoma
2. Age≥18 years
3. ECOG (Eastern Cooperative Oncology Group) 0 or 1
4. At least one site of PSMA-positive disease on a ¹⁸F-piflufolastat PET/CT performed within 3 weeks of trial recruitment
5. Creatinine of ≤1.4 or Creatine clearance of ≥60 mL/minute

### Exclusion Criteria:

1. Known allergy/hypersensitivity to PSMA-targeted imaging agents
2. Other active malignancy, other than the known prostate cancer

### 5.5.5. Supply of Study Drug at the Site

A [⁶¹Cu]Cu-NODAGA-PSMA I&T composition will be produced on demand in a cyclotron facility. Radioisotope will be delivered by courier according to the scheduling of patients for [⁶¹Cu]Cu-NODAGA-PSMA I&T PET/CT. This delivery should require approximately 90 minutes. Upon arrival, the transported box will be received/handled, and calibrated/dispensed for patient administration according to standard protocols for radioactive materials. The transport box will be opened in a designated radioactive hot lab with appropriate shielding. A dose calibrator will be used to measure the appropriate dose of [⁶¹Cu]Cu-NODAGA-PSMA I&T composition for subject administration. The dose will be administered by the by the PI or a nuclear medicine technician authorized by the PI under the guidance of an Authorized User for the administration of medical isotopes.

Subjects will undergo administration of 10 mL of 100-300 MBq (2.7-8.1 mCi) [⁶¹Cu]Cu-NODAGA-PSMA I&T composition intravenously described in Table 20, followed by PET/CT imaging 60 (+/−10) minutes post radiotracer administration.

### 5.5.6. Administration Instructions

An intravenous (IV) line will be placed. The [⁶¹Cu]Cu-NODAGA-PSMA I&T composition will be intravenously administered over approximately 10 seconds.

### 5.5.7. Storage

[⁶¹Cu]Cu-NODAGA-PSMA I&T composition will be received and dispensed on the same day. During the time between receiving and dispensing of the radioisotope, it will be stored in a dedicated, lock-secured, radioisotope hot lab within the Hoag Molecular Imaging and Therapy clinic. The radioisotope will be stored at controlled room temperature 19 to 24° C. (66 to 75° F.) with proper shielding. An accurate and current accounting of the receipt and dispensing each dose of the study radioisotope will be maintained on an ongoing basis by a member of the study site staff. Excess radioisotope which was not administered will be stored in the dedicated hot lab for a minimum of 10 half-lives (33 hours), prior to disposal.

### 5.5.8. Clinical Assessments

Prior to conducting any study-related activities, written informed consent and the Health Insurance Portability and Accountability Act (HIPAA) authorization must be signed and dated by the subject.

Demographic information (date of birth, gender, race) will be recorded at Screening (Visit 1).

Body temperature, blood pressure, pulse, respirations, and oximetry will be performed before administration of radioisotope, as well as 30 minutes and two hours following administration.

Subjects will be monitored during and for 2 hours after tracer administration, and a follow-up phone call the following day will be performed to evaluate for rare side effects.

### 5.5.9. Evaluations by Visit

### Visit 1—Screening and Consent

Review Inclusion and Exclusion Criteria to confirm the subject is appropriate for the study.
Review the study with the subject and obtain written informed consent and HIPAA authorization.
Assign the subject a unique subject number.
Visit 1 may occur in person with the subject signing a physical consent form or by telehealth/telephone with the subject signing an electronic consent form. Table 31 provides summary of study activities.

### Visit 2—[⁶¹Cu]Cu-NODAGA-PSMA I&T Administration and PET/CT Scan

Perform and record vital signs.
Place IV catheter.
Obtain blood for whole blood and plasma radioactivity counts prior to radiotracer administration and at 3-5 time points post radiotracer administration, from the following time ranges
   a. 15 minutes (+/−10 minutes)
   b. 30 minutes (+/−10 minutes)
   c. 60 minutes (+/−15 minutes)
   d. 120-240 minutes
   e. 240-420 minutes
Administer 100-300 MBq (2.7-5.4 mCi) of [⁶¹Cu]Cu-NODAGA-PSMA I&T composition intravenously.
Perform and record vital signs and assess for adverse reactions 30-60 minutes and 120-240 minutes post radiotracer administration.
Perform PET/CT imaging at 2 or 3 time points post radiotracer administration, from the following time ranges.
   a. 60 minutes (+/−15 minutes)
   b. 120-240 minutes
   c. 240-420 minutes
Perform and record vital signs and assess for adverse reactions 120 (+/−10 minutes) post radiotracer administration.
   Visit 3—Day after Phone Call
Record any adverse reactions.

TABLE 31
Summary of Human Clinical Study Activities

| | | Visit 2 (Day 1) | | | | | | | | |
|---|---|---|---|---|---|---|---|---|---|---|
| | Visit 1 | Pre- | | Time 15 | Time 30 | Time | Time 60 | Time | Time | |
| | (Day −30 | adminis- | Time | (+/−5) | (+/−10) | 30-60 | (+/−15 | 120-240 | 240-420 | Visit 3 |
| | to 0) | tration | 0 | min | min | min | min) | min | min | (Day 2) |
| Confirmation | X | | | | | | | | | |
| of Eligibility | | | | | | | | | | |
| and Consent | | | | | | | | | | |
| Vital Signs | | X | | | | X | | X | | |
| Place IV | | X | | | | | | | | |
| Catheter | | | | | | | | | | |
| Blood draw | | X | | X | X | | X | X | X | |
| for dosimetry | | | | | | | | | | |
| Administer | | | X | | | | | | | |
| 61Cu- | | | | | | | | | | |
| NODAGA- | | | | | | | | | | |
| PSMA | | | | | | | | | | |
| Assess for | | | | | | X | | X | | |
| adverse | | | | | | | | | | |
| reactions | | | | | | | | | | |
| PET/CT | | | | | | | X | X | X | |
| Phone Call | | | | | | | | | | X |
| Follow Up | | | | | | | | | | |

### 5.5.10. Adverse Events

An adverse event (AE) is any untoward medical occurrence in a clinical investigation of a patient administered a pharmaceutical product and that does not necessarily have a causal relationship with the treatment. An AE is therefore any unfavorable and unintended sign (including an abnormal laboratory finding), symptom or disease temporally associated with the administration of an investigational product, whether or not related to that investigational product. An unexpected AE is one of a type not identified in nature, severity, or frequency or of greater severity or frequency than expected.

The Investigator will probe, via discussion with the subject, for the occurrence of AEs during each subject visit and record the information in the site's source documents. Adverse events will be recorded in the patient CRF. Adverse events will be described by duration (start and stop dates and times), severity, outcome, treatment and relation to study drug, or if unrelated, the cause.

The National Cancer Institute's Common Terminology Criteria for Adverse Events (CTCAE) Version 3.0 should be used to assess and grade AE severity, including laboratory abnormalities judged to be clinically significant. The modified criteria can be found in the study manual. If the experience is not covered in the modified criteria, the guidelines shown in Table 32 below should be used to grade severity. It should be pointed out that the term “severe” is a measure of intensity and that a severe AE is not necessarily serious.

TABLE 32
AE Severity Grading

| Severity (Toxicity Grade) | Description |
|---|---|
| Mild (1) | Transient or mild discomfort; no limitation in activity; no medical |
| | intervention or therapy required. The subject may be aware of the sign |
| | or symptom but tolerates it reasonably well. |
| Moderate (2) | Mild to moderate limitation in activity, no or minimal medical |
| | intervention/therapy required. |
| Severe (3) | Marked limitation in activity, medical intervention/therapy required, |
| | hospitalizations possible. |
| Life-threatening (4) | The subject is at risk of death due to the adverse experience as it |
| | occurred. This does not refer to an experience that hypothetically |
| | might have caused death if it were more severe. |

The relationship of an AE to the study drug should be assessed using the following the guidelines in Table 33.

TABLE 33
AE Relationship to Study Drug

| Relationship | |
|---|---|
| to Drug | Comment |
| Definitely | Previously known toxicity of agent; or an event that follows a reasonable temporal |
| | sequence from administration of the drug; that follows a known or expected response |
| | pattern to the suspected drug; that is confirmed by stopping or reducing the dosage of |
| | the drug; and that is not explained by any other reasonable hypothesis. |
| Probably | An event that follows a reasonable temporal sequence from administration of the |
| | drug; that follows a known or expected response pattern to the suspected drug; that is |
| | confirmed by stopping or reducing the dosage of the drug; and that is unlikely to be |
| | explained by the known characteristics of the subject's clinical state or by other |
| | interventions. |
| Possibly | An event that follows a reasonable temporal sequence from administration of the |
| | drug; that follows a known or expected response pattern to that suspected drug; but |
| | that could readily have been produced by a number of other factors. |
| Unrelated | An event that can be determined with certainty to have no relationship to the study |
| | drug. |

An SAE is defined as any AE occurring at any dose that results in any of the following outcomes:
death
a life-threatening adverse experience
inpatient hospitalization or prolongation of existing hospitalization
a persistent or significant disability/incapacity
a congenital anomaly/birth defect

Other important medical events may also be considered an SAE when, based on appropriate medical judgment, they jeopardize the subject or require intervention to prevent one of the outcomes listed.

Study sites will document all SAEs that occur (whether or not related to study drug). The collection period for all SAEs will begin after informed consent is obtained and end after procedures for the final study visit have been completed.

In accordance with the standard operating procedures and policies of the local Institutional Review Board (IRB)/Independent Ethics Committee (IEC), the site investigator will report SAEs to the IRB/IEC.

### 5.5.11. Discontinuation and Replacement of Subjects

A subject may be discontinued from study treatment at any time if the subject, the investigator, or the Sponsor feels that it is not in the subject's best interest to continue. The following is a list of possible reasons for study treatment discontinuation:
Subject withdrawal of consent
Subject is not compliant with study procedures
Adverse event that in the opinion of the investigator would be in the best interest of the subject to discontinue study treatment

All subjects are free to withdraw from participation at any time, for any reason, specified or unspecified, and without prejudice.

A subject may be withdrawn from the study at any time if the subject, the investigator, or the Sponsor feels that it is not in the subject's best interest to continue. Subjects who withdraw from the study will be replaced.

### 5.5.12. Protocol Violations

A protocol violation occurs when the subject, investigator, or Sponsor fails to adhere to significant protocol requirements affecting the inclusion, exclusion, subject safety and primary endpoint criteria. Protocol violations for this study include, but are not limited to, the following:
Failure to meet inclusion/exclusion criteria
Failure to administer [⁶¹Cu]Cu-NODAGA-PSMA I&T

Failure to comply with Good Clinical Practice (GCP) guidelines will also result in a protocol violation. The Sponsor will determine if a protocol violation will result in withdrawal of a subject.

Violation Form detailing the violation will be generated. This form will be signed by a Sponsor representative and the Investigator. A copy of the form will be filed in the site's regulatory binder and in the Sponsor's files.

### 5.5.13. Statistical Methods and Considerations

We expect to enroll 6-10 patients in the proposed study. The sample size is exploratory. Demographic data will be tabulated. For the primary objective of safety, side effects will be monitored the day of and the day following radiotracer administration. As the [⁶¹Cu]Cu-NODAGA-PSMA I&T PET composition used in this trial is given at a low, imaging dose, serious adverse events are not expected. If a single serious adverse event is identified, then it will be recorded.

For the secondary objective of dosimetry, dosimetry will be calculated from PET/CT images and radioactive counts in blood samples by an experience medical physicist. Regions of interest from normal organs and tumors will be drawn on PET/CT images. Blood radioactivity counts will be measured over time to obtain an estimate of total body clearance. Dosimetry will be calculated according to the Medical Internal Radiation Dose (MIRD) Committee of the Society of Nuclear Medicine and Molecular Imaging (SNMMI) to calculate radiation absorbed-dose estimates for the whole body and organs.

For the secondary objective of effectiveness, the number of suspected PSMA-positive malignant lesions will be calculated in both the standard-of-care ¹⁸F-Piflufolastat PET/CT and the experimental [⁶¹Cu]Cu-NODAGA-PSMA I&T PET/CT. The percentage of patients with any suspected PSMA-positive malignant lesions will be determined for each radiotracer. To assess whether the distribution of the number of lesions was higher with ¹⁸F-Piflufolastat PET/CT or [⁶¹Cu]Cu-NODAGA-PSMA I&T, 1-sided Wilcoxon signal-rank tests for paired data will be performed. To account for the small sample size, results with a P value of less than 0.1 will be considered statistically significant.

### 5.5.14. Comparison Between Pylarify and [⁶¹Cu]Cu-NODAGA-PSMA I&T

Eight subjects meeting the selection criteria described herein in Example 5 were administered Pylarify at 350 MBq/patient circa and performed PET/CT imaging 1 hour after the radiotracer administration.

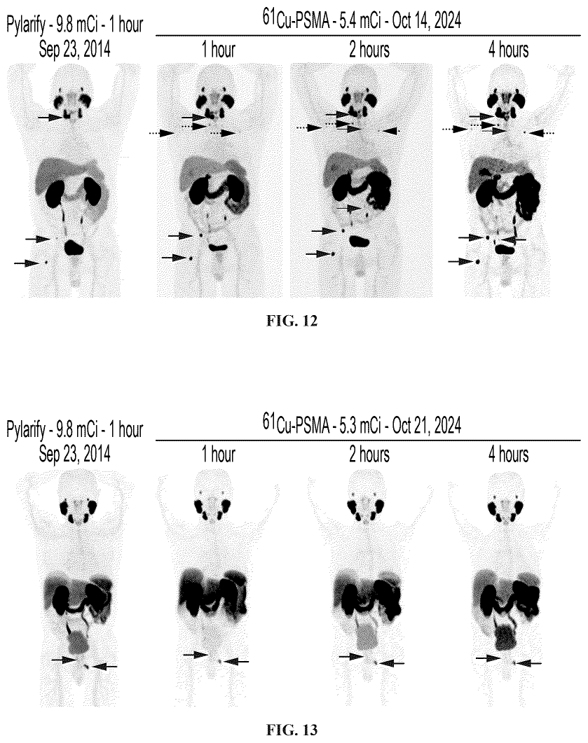

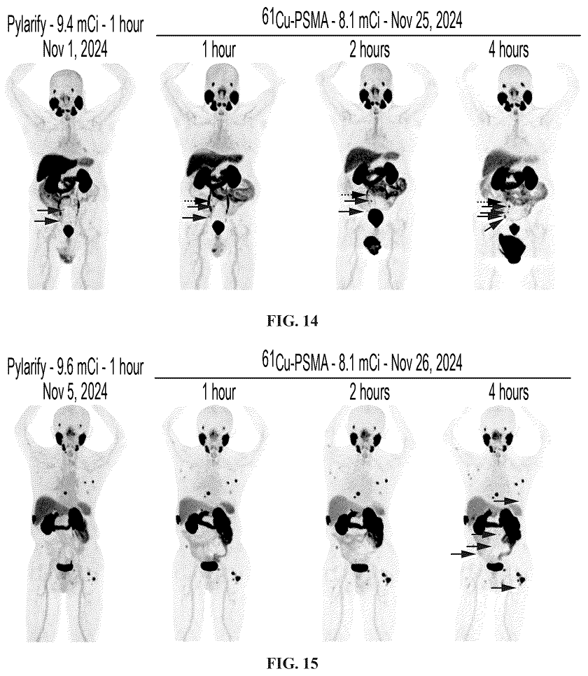

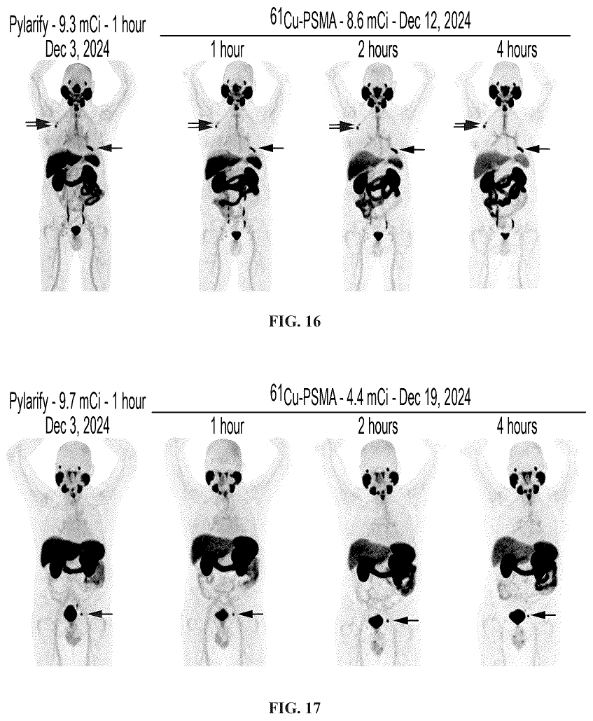

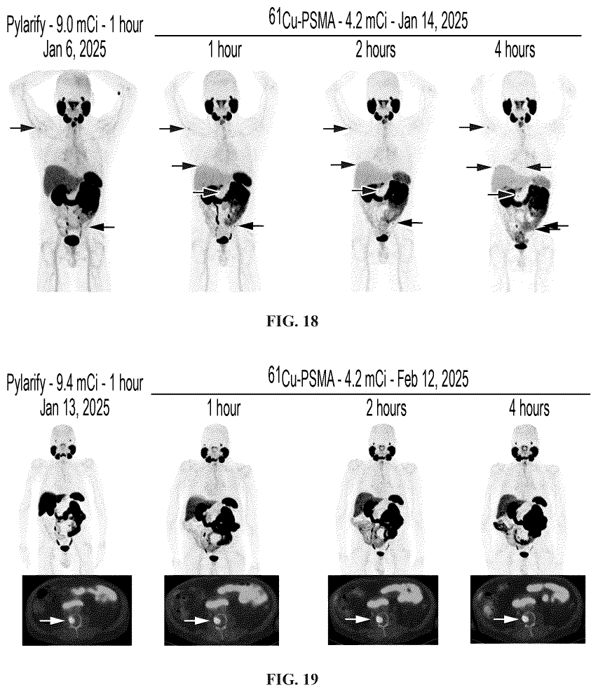

Within 21 days after the Pylarify administration, the same eight subjects were administered [⁶¹Cu]Cu-NODAGA-PSMA I&T by following the protocol described herein in Example 5. Imaging results of lesions and tumor-to-background ratio from Pylarify studies and [⁶¹Cu]Cu-NODAGA-PSMA I&T studies are summarized in Table 34 and Table 35. The PET imaging results of eight subjects are presented in FIGS. 12-19.

TABLE 34
Lesions Detected in Subject After Administration
of Pylarify or [61Cu]Cu-NODAGA-PSMA I&T

| | | Lesions | | | |
|---|---|---|---|---|---|
| | | Detected | New Lesions Detected with | | |
| Subject | | with Pylarify | [⁶¹Cu]Cu-NODAGA-PSMA I&T | | |
| # | Concordance | 1 hour | 1 hour | 2 hour | 4 hour |
| 1 | 100% | 3 | 5 | 7 | 8 |
| 2 | 100% | 2 | 0 | 0 | 0 |
| 3 | 100% | 2 | 1 | 1 | 3 |
| 4 | 100% | 23 | 0 | 2 | 10 |
| 5 | 100% | 3 | 0 | 0 | 0 |
| 6 | 100% | 3 | 0 | 0 | 0 |
| 7 | 100% | 4 | 3 | 3 | 5 |
| 8 | 100% | 1 | 0 | 0 | 0 |

TABLE 35
Tumor-to-Background Ratio of Imaging Results After Administration
of Pylarify or [61Cu]Cu-NODAGA-PSMA I&T

| | Tumor-to- | | | | | | |
|---|---|---|---|---|---|---|---|
| | Background | Tumor-to-Background Ratio with [⁶¹Cu]Cu- | | | | | |
| | Ratio with | NODAGA-PSMA I&T | | | | | |
| Subject | Pylarify | (% difference from Pylarify results at 1 hour) | | | | | |
| # | 1 hour | 1 hour | | 2 hour | | 4 hour | |
| 1 | 53.3 | 59.9 | (12%) | 114.5 | (115%) | 135.5 | (154%) |
| 2 | 3.9 | 5.3 | (37%) | 3.4 | (−13%) | 2.2 | (−43%) |
| 3 | 6.1 | 9.3 | (52%) | 6.7 | (9%) | 23.0 | (274%) |
| 4 | 77.0 | 126.2 | (64%) | 161.4 | (110%) | 152.8 | (98%) |
| 5 | 26.9 | 53.3 | (98%) | 68.3 | (154%) | 70.0 | (160%) |
| 6 | 7.1 | 11.3 | (60%) | 16.0 | (126%) | 23.3 | (230%) |
| 7 | 3.9 | 5.6 | (45%) | 6.6 | (70%) | 15.0 | (287%) |
| 8 | 26.8 | 24.2 | (−10%) | 34.5 | (29%) | 59.7 | (123%) |

On average, [⁶¹Cu]Cu-NODAGA-PSMA I&T (aka ⁶¹Cu-PSMA) displayed 90% higher tumor-to-background ratio than Pylarify. Due to better imaging, higher detection rate of metastatic lesions was also observed for ⁶¹Cu-PSMA. The detection of additional pelvic metastatic lesions (e.g., in subject #3) allows potential change to systemic therapy instead of external beam radiation therapy (EBRT). Improved lesion detection in later imaging (e.g., 4 hours) makes 4-hour imaging interval possible, offers logistical advantages and aligns well with 3-hour interval used in routine bone scintigraphy.

### 5.6. Example 6: Exemplary Method of Measuring Apparent Molar Activity

A [⁶¹Cu]CuCl₂ solution may be characterized by apparent molar activity (AMA). Apparent molar activity assesses, for example, competing trace metal impurities of ⁶¹Cu chelation by quantifying the minimum amount of a given chelator required for efficient radiolabeling.

An AMA test measures the percentage of complexation, by radio-TLC, after titrating a quantity of [⁶¹Cu]CuCl₂ solution with different amounts of a chelator (e.g., DOTA, NODAGA, etc.).

The results were then plotted (x-axis (logarithmic): nmol of chelator; y-axis: % of complexation). The lowest nmol value of chelator corresponding to ≥95% complexation achieved was recorded.

The experimental AMA value was then calculated according to the formula:

$$AMA value ( experimental ) = \frac{Activity [ MBq ]}{{n}_{NODAGA} [ nmol ]}$$

where:
Activity: indicates the activity present in the fixed amount of [⁶¹Cu]CuCl₂ solution used for the titration decay corrected at the EoP (end of production; end of bombardment plus 1 hour).
n_{NODAGA}: indicates the lowest nmol value of chelator for which≥95% complexation was achieved.

This test was performed to assess, for example:
1) the grade of a chemical employed for the manufacturing of the [⁶¹Cu]CuCl₂ solution;
2) the quality of a consumable employed for the manufacturing of the [⁶¹Cu]CuCl₂ solution;
3) the target coin manufacturing process; or
4) in case poor radiolabeling yields were observed.

### AMA Test Procedure

The mobile phase was prepared as (0.1M sodium citrate) as follows:
   Weigh 5.882 g of trisodium citrate dihydrate.
   Add Suprapur water up to a volume of 200 mL.
   Adjust the pH by adding HCl dropwise until it reaches a pH of 5.
Transfer an adequate amount of mobile phase into the TLC development chamber to cover a depth of 5 mm.
Prepare the sodium acetate solution (0.5 M in Ultrapur water) as follows:
   Weigh 2.051 g of sodium acetate.
   Add Ultrapur water up to a volume of 50 mL.
   Apply 10 μL of the solution on a pH strip and check if the pH was 8.
A 5 mM stock solution of NODAGA was prepared in 0.5 M sodium acetate (Stock 1) as follows:
   Weigh 3.1 mg of NODAGA.
   Add Ultrapur water or equivalent up to a volume of 1.5 mL.
   Store at −20° C. after use.
A 50 μM stock solution of NODAGA was prepared in 0.5 M sodium acetate (Stock 2) by mixing 10 μL of Stock 1 with 990 μL 0.5 M sodium acetate. Store at −20° C. after use.
NODAGA test solutions S1-S2 were prepared for chelator titration as displayed in Table T1 by diluting Stock 2 with the listed volumes of sodium acetate 0.5 M. The test solution S0 was a blank solution (without the addition of NODAGA) of 100 μL of 0.5 M sodium acetate.

TABLE T1
Preparation of solution 1-3 for chelator titration.

| Solution | c_{NODAGA} | Volume | Volume sodium |
|---|---|---|---|
| name | [nM] | stock 2 [μL] | acetate [μL] |
| S0 | 0 | 0 | 100 |
| S 1 | 3000 | 100 | 1567 |
| S 2 | 1500 | 50 | 1617 |
| S 3 | 900 | 30 | 1637 |

The test solutions, S4-S10, 1:10 dilutions of the S1-S7 were prepared with 0.5 M sodium acetate according to the dilution scheme shown in Table T2.

TABLE T2
Preparation of solution 4-10 for chelator titration.

| Solution | c_{NODAGA} | Volume | Volume sodium |
|---|---|---|---|
| name | [nM] | Sx [μL] | acetate [μL] |
| S 4 | 300 | 100 of S1 | 900 |
| S 5 | 150 | 100 of S2 | 900 |
| S 6 | 90 | 100 of S3 | 900 |
| S 7 | 30 | 100 of S4 | 900 |
| S 8 | 15 | 100 of S5 | 900 |
| S 9 | 9 | 100 of S6 | 900 |
| S 10 | 3 | 100 of S7 | 900 |

A solution of 0.05 M HCl was prepared as follows:
   Add 5 mL of Ultrapur water in a 15 mL Falcon tube.
   Add 53 μL of 30% HCl to the falcon using a micropipette.
   Add Ultrapur water up to 10 mL.
   Spot 10 μL of the solution on a pH strip and confirm the pH is between 1.0 and 1.6.
The [⁶¹Cu]CuCl₂ solution was diluted with 0.05 M hydrochloric acid to reach a total volume of 1 mL with an activity concentration of 0.2 MBq/μL (EoP) according to the following formula:

$${[}^{61} Cu ] {CuCl}_{2} solution [ μ L ] = \frac{0.2 [ MBq / μ L ]}{A C ( EoP ) [ MBq / μ L ]} * 1000 μ L$$

$$0.05 M hydrchloric acid for dilution = 1000 μ L - {[}^{61} Cu ] {CuCl}_{2} solution to draw [ μ L ] where A C ( EoP ) is activity concentration at EoP .$$

The solution were named “diluted Cu-61 solution”. See the example in Table T3.

TABLE T3
[⁶¹Cu]CuCl₂ dilution examples.

| | | Activity | |
|---|---|---|---|
| Activity | | concentration | |
| eluted at | Volume | at EoP | |
| EoP [MBq] | [mL] | [MBq/μL] | Dilution for AMA test |
| 1000 | 3.0 | 0.33 | 600 μL of [⁶¹Cu]CuCl₂ |
| | | | diluted with 400 μL of |
| | | | 0.05M HCl |
| 2000 | 3.0 | 0.67 | 300 μL of [⁶¹Cu]CuCl₂ |
| | | | diluted with 700 μL of |
| | | | 0.05M HCl |
| 3500 | 3.0 | 1.17 | 233 μL of [⁶¹Cu]CuCl₂ |
| | | | diluted with 767 μL of |
| | | | 0.05M HCl |

Add in each solution for chelator titration 50 μL of diluted [⁶¹Cu]CuCl₂ solution (as prepared in Table T3), obtaining the reaction solutions listed in Table T4.

TABLE T4
Reaction solutions.

| | | Volume of | Volume | | | |
|---|---|---|---|---|---|---|
| Reaction | | NODAGA | of ⁶¹Cu | Finale | | |
| solution | NODAGA | solution Sx | solution | volume | n_{NODAGA} | c_{NODAGA} |
| name | solution | [μL] | [μL] | [μL] | [nmol] | [nM] |
| RS 1 | S1 | 100 | 50 | 150 | 0.3 | 2000 |
| RS 2 | S2 | 100 | 50 | 150 | 0.15 | 1000 |
| RS 3 | S3 | 100 | 50 | 150 | 0.09 | 600 |
| RS 4 | S4 | 100 | 50 | 150 | 0.03 | 200 |
| RS 5 | S5 | 100 | 50 | 150 | 0.015 | 100 |
| RS 6 | S6 | 100 | 50 | 150 | 0.009 | 60 |
| RS 7 | S7 | 100 | 50 | 150 | 0.003 | 20 |
| RS 8 | S8 | 100 | 50 | 150 | 0.0015 | 10 |
| RS 9 | S9 | 100 | 50 | 150 | 0.0009 | 6 |
| RS 10 | S10 | 100 | 50 | 150 | 0.0003 | 2 |
| RS 0 | S0 | 100 (Only 100 | 50 | 150 | 0 | 0 |
| | | μL of sodium | | | | |
| | | acetate 0.5M) | | | | |

The reaction solutions were incubated at room temperature for 5 minutes.
After incubation, perform TLC measurement on all the reaction solutions as follows:
   Using a micropipette, spot 2 μL of each [⁶¹Cu]Cu-NODAGA reaction solution RS0-RS10 onto different pre-cut TLC paper.
   Allow the TLC paper to dry for about 5 minutes or until there was no visible stain.
   Place the TLC paper in the TLC chamber with the stained side on the bottom of the chamber. When the solvent front was about 1 cm from the top of the strip, remove the TLC paper from the TLC chamber with tweezers.
   Mark the solvent front on the TLC paper strip with a pencil.
   Measure the distance between the starting line and the solvent front with a ruler and let the strip dry completely.
After scanning the TLC plate, the software (e.g., Biochrom) will generate an evaluation report. Print the evaluation report and record the results. All data (i.e., RF, % of total activity, % of ROI) of the peaks must be documented.
Using the regions of interest (ROI) technique (drawing regions over distinct areas of activities), the % of complexation of [⁶¹Cu]Cu-NODAGA was expressed as a percentage of the total detected activity, as follows:

$${Complexation}_{[ 61 C u ] C u - NODAGA} ( \% ) = \frac{{Activity}_{{[}^{61} C u ] C u - NODAGA} [ counts ]}{Total activity [ counts ]} * 100$$

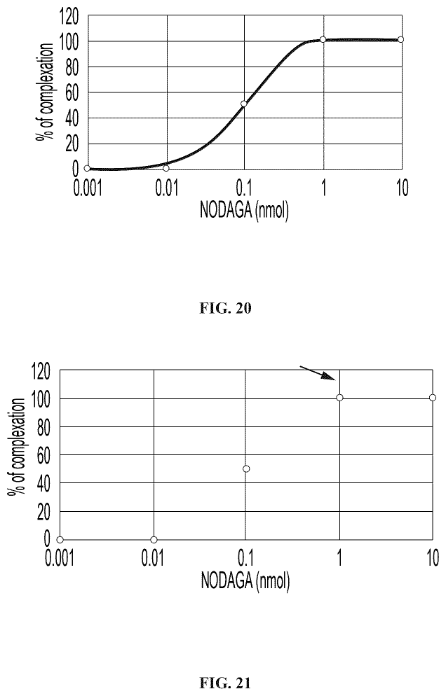

The TLC scanner results were plotted (x-axis (logarithmic): nmol of NODAGA chelator; y-axis: % of complexation) and will follow a sigmoidal trend. An example of plot was given in FIG. 20.
The lowest value of nmol of chelator in correspondence of which≥95% complexation is identified on the plot. An example was reported in FIG. 21.

The experimental AMA value was then calculated according to the formula:

$$AMA value ( experimental ) = \frac{Activity [ MBq ]}{{n}_{NODAGA} [ nmol ]}$$

where:
Activity: indicates the activity present in the fixed amount of [⁶¹Cu]CuCl₂ solution used for the titration decay corrected at the EoP.
n_{NODAGA}: indicates the lowest value of nmol of chelator where ≥95% complexation was achieved.

### 6. EQUIVALENTS AND INCORPORATION BY REFERENCE

While aspects of this disclosure have been particularly shown and described with reference to a preferred embodiment and various alternate embodiments, it will be understood by persons skilled in the relevant art that various changes in form and details can be made therein without departing from the scope of the disclosure.

All references, issued patents, and patent applications cited within the body of the instant specification are hereby incorporated by reference in their entirety, for all purposes. In particular, U.S. Provisional Patent Application No. 63/568,830 (filed Mar. 22, 2024) and International Application Nos. PCT/US2023/75064 (filed Sep. 25, 2023), PCT/US2023/75067 (filed Sep. 25, 2023), and PCT/US2025/21198 (filed Mar. 24, 2025) are hereby incorporated by reference in their entirety.

1. A method for imaging a subject comprising:
(a) administering an effective amount of a pharmaceutical composition to a subject suspected of having or diagnosed with a prostate-specific membrane antigen (PMSA)-expressing tumor, wherein the pharmaceutical composition comprises a radiotracer that has the structure 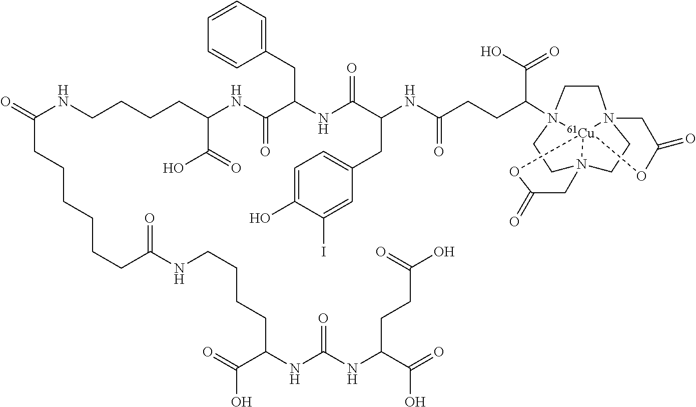
or is a pharmaceutically acceptable salt thereof;
wherein the pharmaceutical composition is characterized by a radiotracer radiochemical purity of ≥97% at 12 hours after end of synthesis and one or more of:
a [⁶¹Cu]Cu radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a ¹⁰⁹Cd specific activity≤0.1 Bq/g; and
(b) generating one or more radiographic images of the subject.

2. (canceled)

3. A method for determining a subject's response to a cancer treatment comprising:
(a) administering an effective amount of a pharmaceutical composition to a subject diagnosed with a PSMA-expressing tumor at an earlier time point and at a later time point, wherein the pharmaceutical composition comprises a radiotracer that has the structure 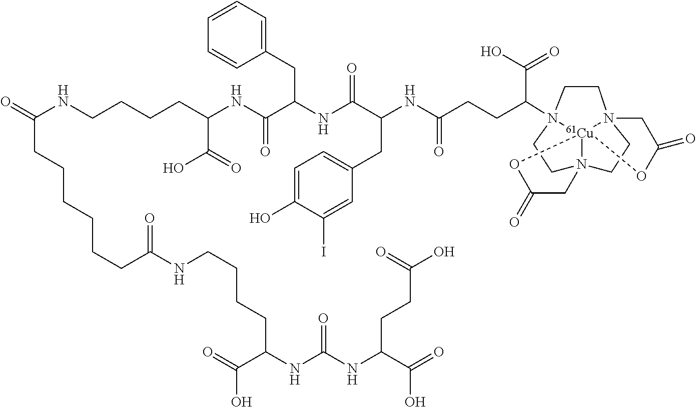
or is a pharmaceutically acceptable salt thereof;
wherein the pharmaceutical composition is characterized by a radiotracer radiochemical purity of ≥97% at 12 hours after end of synthesis and one or more of:
a [⁶¹Cu]Cu radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a 109Cd specific activity≤0.1 Bq/g;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

4-76. (canceled)

77. A pharmaceutical composition comprising a radiotracer that has the structure 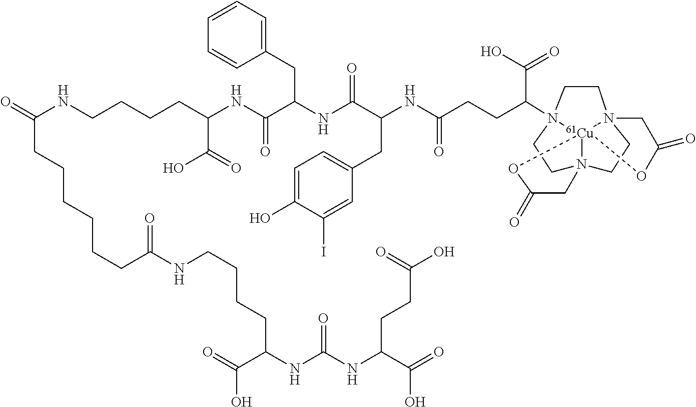
or is a pharmaceutically acceptable salt thereof;
wherein the pharmaceutical composition is characterized by a radiotracer radiochemical purity of ≥97% at 12 hours after end of synthesis and one or more of:
a [⁶¹Cu]Cu radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a ¹⁰⁹Cd specific activity≤0.1 Bq/g.

78. The pharmaceutical composition according to claim 77, wherein the activity of the pharmaceutical composition is ≥25 MBq.

79. (canceled)

80. The pharmaceutical composition according to claim 77, wherein the pharmaceutical composition is characterized by a radiotracer radiochemical purity≥99% at 12 hours after end of synthesis.

81. The pharmaceutical composition according to claim 77, wherein the pharmaceutical composition is characterized by a ⁶¹[Cu]Cu radionuclidic purity of the pharmaceutical composition at end of synthesis of ≥98%.

82. The pharmaceutical composition according to claim 77, wherein the pharmaceutical composition is characterized by a radiocobalt activity content at end of synthesis of ≤0.01%.

83. The pharmaceutical composition according to claim 77, wherein the pharmaceutical composition is characterized by at least one of a ⁵⁶[Co]Co specific activity or ⁵⁸[Co]Co specific activity of ≤1,500 Bq/g.

84-86. (canceled)

87. The pharmaceutical composition according to claim 77, wherein the pharmaceutical composition is characterized by one or more of: a ^{110m}Ag specific activity≤0.1 Bq/g, a ^{108m}Ag specific activity≤0.1 Bq/g, or a ¹⁰⁹Cd specific activity≤0.1 Bq/g.

88. The pharmaceutical composition according to claim 77, wherein the sum of the specific activities of the radionuclidic impurities in the pharmaceutical composition is ≤8,000 Bq/g.

89. The pharmaceutical composition according to claim 77, wherein the pharmaceutical composition is characterized by one or more of: Al≤1.2 ng/MBq, Co≤0.2 ng/MBq, Fe≤1.7 ng/MBq, Pb≤0.8 ng/MBq, or Zn≤0.8 ng/MBq.

90. The pharmaceutical composition according to claim 77, wherein the activity concentration of the pharmaceutical composition is ≥10 MBq/mL.

91. (canceled)

92. The pharmaceutical composition according to claim 77, wherein the apparent molar activity of the radiotracer is ≥1 MBq/nmol.

93. The pharmaceutical composition according to claim 77, wherein the radiotracer is present in an amount ≥1 μg.

94. (canceled)

95. The pharmaceutical composition according to claim 77, further comprising a radiolytic inhibitor.

96. The pharmaceutical composition according to claim 95, wherein the radiolytic inhibitor is selected from ascorbic acid, gentisic acid, citric acid, N-tert-butyl-α-phenylnitrone (PBN), polyvinylpyrrolidone (PVP), ethanol, DMSA, cysteine, vanillin, methionine, adenine, dobesilic acid, thymine, uracil, nicotinic acid, nicotinamide, salts of any of the foregoing, and combinations thereof.

97. The pharmaceutical composition according to claim 95, wherein the radiolytic inhibitor is ascorbic acid or a salt thereof, preferably ascorbic acid.

98. The pharmaceutical composition according to claim 77, further comprising sodium chloride.

99. (canceled)

100. The pharmaceutical composition according to claim 77, wherein the pharmaceutical composition is in the form of a liquid.

101. The pharmaceutical composition according to claim 77, wherein the pharmaceutical composition comprises an aqueous vehicle.

102. The pharmaceutical composition according to claim 101, wherein the total volume of the composition is from 2 to 15 mL.

103. The pharmaceutical composition according to claim 95, wherein the radiolytic inhibitor is in a concentration of ≥3 μg/mL.

104. (canceled)

105. The pharmaceutical composition according to claim 98, wherein the sodium chloride is in a concentration from 0.5 mg/mL to 50 mg/mL.

106. (canceled)

107. The pharmaceutical composition according to claim 101, comprising:
1-100 μg of the radiotracer;
3 μg/mL to 15 μg/mL of a radiolytic inhibitor; and
1 mg/mL to 10 mg/mL sodium chloride.

108. The pharmaceutical composition according to claim 101, wherein the pH of the pharmaceutical composition is from 5 to 7.

109. The pharmaceutical composition according to claim 77, wherein the radiotracer has the structure: 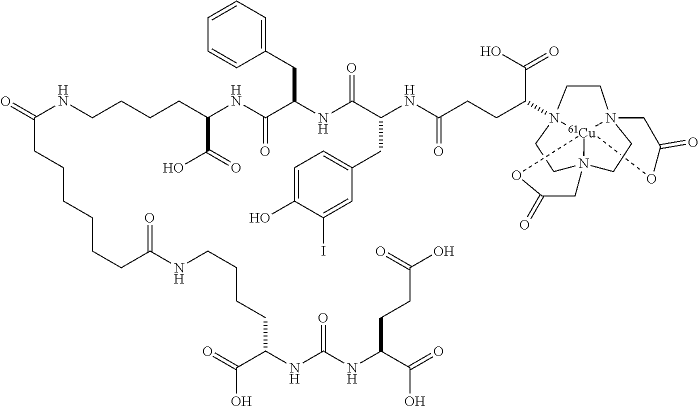
or is a pharmaceutically acceptable salt thereof.

110. The pharmaceutical composition according to claim 77, further comprising a pH stabilizer.

111. The pharmaceutical composition according to claim 110, wherein the pH stabilizer is sodium acetate.

112. The pharmaceutical composition according to claim 111, wherein the sodium acetate is in a concentration from 5 mg/mL to 50 mg/mL.

113. The pharmaceutical composition according to claim 101, wherein the aqueous vehicle comprises isotonic saline solution.
